(19) United States
(12) Patent Application Publication
(10) Pub. No.: US 2008/0050358 A1
(43) Pub. Date: Feb. 28, 2008

(54) Identification of Snps Associated with Hyperlipidemia, Dyslipidemia and Defective Carbohydrate Metabolism

(76) Inventors: Leena Peltonen-Palotie, Helsinki (FI); Marja-Riita Taskinen, Helsinki (FI); Paivi Pajukanta, Los Angeles, CA (US); Christian Ehnholm, Kauniainen (FI)

Correspondence Address: BIRCH STEWART KOLASCH & BIRCH, PO BOX 747, FALLS CHURCH, VA, 22040-0747, US

(21) Appl. No.: 10/590,043

(22) PCT Filed: Feb. 17, 2005
(86) PCT No.: PCT/EP05/01624

§ 371 Date: Feb. 1, 2007

(30) Foreign Application Priority Data
Feb. 17, 2004 (EP) 04003554.5

(51) Int. Cl. A61K 39/395; A01H 5/00; A01K 67/027; C07H 21/04; C12N 1/19; C12N 15/63; C12N 5/06; C12Q 1/68; G01N 33/92; C12N 5/10; C12N 5/04; C12N 1/21; C12N 1/15; A61K 31/7052; A61K 38/16

(52) U.S. Cl. 424/130.1; 435/252.3; 435/254.11; 435/254.2; 435/320.1; 435/325; 435/348; 435/419; 435/6; 436/71; 514/12; 514/44; 514/789; 536/23.1; 536/24.33; 800/13; 800/295

(57) ABSTRACT

The present invention relates to a nucleic acid molecule comprising a chromosomal region contributing to or indicative of hyperlipidemias and/or dyslipidemias or defective carbohydrate metabolism, wherein said nucleic acid molecule is selected from the group consisting of: (a) a nucleic acid molecule having or comprising the nucleic acid sequence of SEQ ID NO: 1, wherein said nucleic acid sequence has one or more mutations having an effect on USFI function; (b) a nucleic acid molecule having or comprising the nucleic acid sequence of SEQ ID NO: 1, wherein said nucleic acid sequence is characterized by comprising a guanine or an adenine residue in position 3966 in intron 7 of the USF1 sequence; and/or (c) a nucleic acid molecule having or comprising the nucleic acid sequence of SEQ ID NO: 1, wherein said nucleic acid sequence is characterized by comprising a cytosine or a thymine residue in position 5205 in, exon 11 of the USF1 sequence; wherein said nucleic molecule extends, at a maximum, 50000 nucleotides over the 5′ and/or 3′ end of the nucleic acid molecule of SEQ ID NO: 1. The present invention further relates to a diagnostic composition comprising a nucleic acid molecule encoding USF1 or a fragment thereof, the nucleic acid molecule disclosed herein, the vector, the primer or primer pair of the present invention or an antibody specific for USF1. Finally, the present invention relates to the use of the nucleic acid molecule of the invention for the preparation of a pharmaceutical composition for the treatment of hyperlipidemia, dyslipidemia, coronary heart disease, type II diabetes, metabolic syndrome, hypertension or atherosclerosis.

The present invention relates to a nucleic acid molecule comprising a chromosomal region contributing to or indicative of hyperlipidemias and/or dyslipidemias and/or defective carbohydrate metabolism, wherein said nucleic acid molecule is selected from the group consisting of: (a) a nucleic acid molecule having or comprising the nucleic acid sequence of SEQ ID NO: 1, wherein said nucleic acid sequence has one or more mutations having an effect on USF1 function; (b) a nucleic acid molecule having or comprising the nucleic acid sequence of SEQ ID NO: 1, wherein said nucleic acid sequence is characterized by comprising a guanine or an adenine residue in position 3966 in intron 7 of the USF1 sequence; and/or (c) a nucleic acid molecule having or comprising the nucleic acid sequence of SEQ ID NO: 1, wherein said nucleic acid sequence is characterized by comprising a cytosine or a thymine residue in position 5205 in exon 11 of the USF1 sequence; wherein said nucleic molecule extends, at a maximum, 50000 nucleotides over the 5′ and/or 3′ end of the nucleic acid molecule of SEQ ID NO: 1. The present invention further relates to a diagnostic composition comprising a nucleic acid molecule encoding USF1 or a fragment thereof, the nucleic acid molecule disclosed herein, the vector, the primer or primer pair of the present invention or an antibody specific for USF1. Finally, the present invention relates to the use of the nucleic acid molecule of the invention for the preparation of a pharmaceutical composition for the treatment of hyperlipidemia, dyslipidemia, coronary heart disease, type II diabetes, metabolic syndrome, hypertension or atherosclerosis.

A variety of documents is cited throughout this specification. The disclosure content of these documents, including manufacturer's manuals and catalogues, is herewith incorporated by reference.

Familial combined hyperlipidemia (FCHL) is characterized by elevated levels of serum total cholesterol (TC), triglycerides (TG), or both^{1,2}. Recently, the first major locus for FCHL was identified on human chromosome 1q21-q23 in 31 Finnish FCHL families⁴. This finding has been replicated in FCHL families from other, more heterogeneous populations⁵⁻⁷. In addition, genome-wide scans have identified several other putative loci for FCHL in Finnish and Dutch study samples⁸⁻⁹. Interestingly, the same markers in the 1q21 region have also been linked to type 2 diabetes mellitus (T2DM) in numerous studies¹⁰⁻¹⁴, including a Finnish study¹⁵. The evidence for linkage obtained for 1q21 has varied in these FCHL and T2DM studies, most likely reflecting genetic heterogeneity as well as population-based and diagnostic differences. Importantly, however, many of the critical metabolic features of FCHL, e.g. hypertriglyceridemia and insulin resistance, also represent trait components of T2DM. Interestingly, a rodent locus for combined hyperlipidemia was linked to a region on mouse chromosome 3, potentially orthologous with human 1q21 (ref. 16). The underlying gene, thioredoxin interacting protein (TXNIP), was recently identified providing a strong positional candidate for human FCHL¹⁷.

As pointed out above, familial combined hyperlipidemia (FCHL) is characterized by elevated levels of serum total cholesterol (TC), triglycerides (TG), or both^{1,2}. This complex disorder is the most common familial hyperlipidemia with a prevalence of 1% to 2% in Western populations¹. FCHL constitutes a powerful genetic factor in atherosclerosis since it is observed in about 20% of coronary heart disease (CHD) patients under 60 years³. Despite tremendous efforts to identify the molecular mechanisms underlying FCHL, its etiology remains unknown. As a consequence it is presently not possible to diagnose or treat patients affected by familial combined hyperlipidemia (FCHL).

In view of the above, the technical problem underlying the present invention was to provide means and methods that allow for an accurate and convenient diagnosis of hyperlipidemias and/or dyslipidemias or defective carbohydrate metabolism or of a predisposition to these conditions.

The solution to said technical problem is achieved by the embodiments characterized in the claims.

Thus, the present invention relates to a nucleic acid molecule comprising a chromosomal region contributing to or indicative of hyperlipidemias and/or dyslipidemias or defective carbohydrate metabolism, wherein said nucleic acid molecule is selected from the group consisting of: (a) a nucleic acid molecule having or comprising the nucleic acid sequence of SEQ ID NO: 1, wherein said nucleic acid sequence has one or more mutations having an effect on USF1 function; (b) a nucleic acid molecule having or comprising the nucleic acid sequence of SEQ ID NO: 1, wherein said nucleic acid sequence is characterized by comprising a guanine or an adenine residue in position 3966 in intron 7 of the USF1 sequence; and/or (c) a nucleic acid molecule having or comprising the nucleic acid sequence of SEQ ID. NO: 1, wherein said nucleic acid sequence is characterized by comprising a cytosine or thymine residue in position 5205 in exon 11 of the USF1 sequence; wherein said nucleic molecule extends, at a maximum, 50000 nucleotides over the 5′ and/or 3′ end of the nucleic acid molecule of SEQ ID NO: 1. In preferred embodiments, the nucleic acid molecule extends up to 40000 nucleotides or up to 25000 nucleotides or up to 5000 nucleotides over the 5′ and/or 3′ end of the nucleic acid molecule of SEQ ID NO: 1.

The term “hyperlipidemias and dyslipidemias” refers to diseases associated with an increased levels of serum total cholesterol and/or triglycerides, as well as increased levels of low-density lipoprotein (LDL) cholesterol and/or apolipoprotein B and/or decreased levels of serum high-density lipoprotein (HDL) cholesterol and/or small dense LDL. In accordance with the present invention such diseases include familial combined hyperlipidemia (FCHL), hypercholesterolemia, hypertriglyceridemia, hypoalphalipoproteinemia, hyperapobetalipoproteinemia (hyperapoB), familial dyslipidemic hypertension (FDH), hypertension, coronary heart disease and atherosclerosis.

In accordance with the invention, the term “defective carbohydrate metabolism” refers to glucose intolerance and insulin resistance. Defective carbohydrate metabolism might therefore be indicative of diseases such as type 2 diabetes mellitus (T2DM) and metabolic syndrome.

The term “contributing to or indicative of hyperlipidemias and/or dysiipidemias or defective carbohydrate metabolism”, refers to the fact that the SNPs and thus the corresponding nucleic acid molecules found are indicative of the condition and possibly also causative therefore. Accordingly, this term necessarily requires that the recited position is indicative of the condition. Said term, on the other hand, does not necessarily require that the particular position containing the SNP is actually causative or contributes to the condition. Yet, said term does not exclude a causative or contributory role of either or both SNPs.

The nucleotide sequence designated SEQ ID NO:1 is a genomic nucleotide sequence of 5687 bp, representing USF1 as deposited under databank accession number RefSeq: NM_{—}007122 for the human USF1 mRNA with the corresponding genomic sequence as deposited under >hg16_refGene_NM_{—}007122 range=chr1:158225833-158231519 in the UCSC Genome Browser on Human in July 2003. For the purpose of the present invention, the activity or function of the polypeptide encoded by this nucleotide sequence is defined as “wild-type USF1 protein activity”. Likewise, SEQ ID NO:1 is understood as representing wild-type USF1 if sequence position 3966 is an adenine and sequence position 5205 is a thymine. USF1 is known as a transcription factor, capable of binding to the recognition sequence CACGTG termed E box and capable of regulating the expression of genes such as apolipoproteins CIII (APOC3), AII (APOA2), APOE, hormone sensitive lipase (LIPE), fatty acid synthase (FAS), glucokinase (GCK), glucagon receptor (GCGR), ATP-binding cassette, subfamily A (ABCA1), renin (REN) and angiotensinogen (AGT). Moreover, USF1 is known to interact with other factors of the cellular transcription machinery, such as USF2.

The term “(poly)peptide” as used herein refers alternatively to peptide or to (poly)peptides. Peptides conventionally are covalently linked amino acids of up to 30 residues, whereas polypeptides (also referred to herein as “proteins”) comprise 31 and more amino acid residues.

The term “one or more mutations having an effect on USF1 function” refers to mutations affecting USF1 function. Throughout the present invention the term “function” and “activity” are used exchangeable. Since USF1 is a transcription factor, the term “USF1 function” refers to its activity as a transcription factor including its specificity to its target recognition sequence on the genomic DNA, its protein interaction sequences and its capability of modulating or regulating transcription. It is important to note, however, that also mutations outside of the coding region of USF1 can have an effect on USF1 function. Such mutations are, for example, mutations affecting the amount of USF1 transcribed in a cell (including mutations affecting promoter activity) or mutations that have an impact on splicing or intracellular transport of the RNA transcripts. Any of these mutations is also comprised by the present invention.

The term “nucleic acid molecule” refers both to naturally and non-naturally occurring nucleic acid molecules. Non-naturally occurring nucleic acid molecules include cDNA as well as derivatives such as PNA.

The term “nucleic acid molecule [ . . . ] comprising the nucleic acid sequence of SEQ ID NO:”, as used throughout this specification, refers to nucleic acid molecules that are at least 1 nucleotide longer than the nucleic acid molecule specified by the SEQ ID NO. At the same time, these nucleic acid molecules extend, at a maximum, 50000 nucleotides over the 5′ and/or 3′ end of the nucleic acid molecule of the invention specified e.g. by the SEQ ID NO: 1.

A number of previous studies in mammalia have tried to identify chromosomal regions contributing to or associated with familial combined hyperlipidemia. A rodent locus for combined hyperlipidemia was linked to a region on mouse chromosome 3, potentially orthologous with human 1q21 (ref. 16). The underlying gene, thioredoxin interacting protein (TXNIP), was recently identified providing a strong positional candidate for human FCHL¹⁷. Surprisingly, the results disclosed by the present invention show that two single-nucleotide polymorphisms located in intron 7 and exon 11, respectively, of human USF1 are associated with hyperlipidemias, dyslipidemias and defective carbohydrate metabolism. The disclosed polymorphisms allow to screen individuals for a presence or predisposition of hyperlipidemia and/or dyslipidemia and/or defective carbohydrate metabolism.

Here we investigated the non-coding SNPs, reported to characterize the alleles associated with FCHL and several component traits of the metabolic syndrome^{6A,7A} (Ng, M. C. Y. et al., manuscript submitted); We observed that the DNA sequence containing the strongest associating SNP usf1s2 was conserved across species and binds protein(s) of nuclear extract, as shown by its ability to produce a mobility shift in an EMSA experiment. In addition to this in vitro evidence, we were able to see differential expression of downstream genes of USF1 in the adipose tissue of 19 individuals depending on whether they carried either the risk or the non-risk allele of the SNP usf1s2.

Transcription factors bind to very specific nucleotide sequences characterized by a short core-sequence of about 4-6 bp flanked by a variable number of degenerate nucleotides. The sequence around usf1s2 in intron 7 agrees well with these criteria showing the perfect cross-species conservation of 5 bp. Our EMSA results lend strong evidence supporting the finding that the sequence surrounding usf1s2 truly represents a functional element. We earlier reported that a 268 bp segment that included this conserved DNA motif enhanced expression of a reporter gene and only in the correct orientation^{6A}. This speaks strongly for the cis-regulatory role of this intronic sequence. This to our knowledge is the first demonstration of a regulatory element of the USF1 gene. The EMSA is a purely in vitro assay in which the DNA sequence under study is in essence naked and is tested in the absence of its normal cellular environment with all its transcriptional machinery and host of other regulatory elements. Some of these interacting elements can be found at a significant distance and would not be present in the probe used for an EMSA. Any tissue-specific effects would also be abolished in the in vitro assay. However, our data from the expression profiles of USF1 regulated genes in fat would indicate an allele specific difference in the expression pattern of these genes and would imply an allele-specific difference in the function of USF1.

We analyzed the known downstream genes of USF1 for possible changes in expression. As the transcriptional regulation of genes is usually the fine tuned result of a concert of various transcription factors and enhancers/repressors that depend on the tissue and different hormonal/environmental cues, it isn't expected that a change in any single factor would have a dramatic effect. Yet, we found the USF1-regulated genes APOE (ref. 13A), ABCA1 (ref. 14A) and AGT (ref. 15A) being significantly differentially regulated depending on the specific allele at the SNP usf1s2. All three genes are highly relevant to the dyslipidemic phenotype. ABCA1 is involved in the first step of the reverse transport of cholesterol by mediating the efflux of phospholipids and cholesterol from macrophages to the nascent HDL particles^{22A}. Loss of function alleles of ABCA1 have been shown to result in Tangier's disease and familial hypoalphalipoproteinemia^{23A}, characterized by very low HDL levels. AGT is an essential component in the control of blood pressure and volume by regulating the amount of water absorption by the kidneys, among other things. APOE facilitates the removal of chylomicron and VLDL remnants from the circulation via the LDL receptor related protein (LRP) mediated endocytosis in the liver^{24A-26A}. APOE has a high affinity to the LDL receptor and an over-expression of APOE results in marked reduction in plasma low density lipoproteins^{27A}. A reduction in APOE thus leads to an accumulation and increased residence time of cholesterol-rich chylomicron and VLDL remnants in circulation—a highly atherogenic phenotype^{24A,28A}. Defects in APOE have also been shown to result in familial dysbetalipoproteinemia with impaired clearance of cholesterol and triglycerides from plasma^{29A,30A}. Recent evidence suggests that APOE has also a critical role in intracellular lipid metabolism. The recycling of APOE from triglyceride rich lipoproteins (TRL) is critical for HDL metabolism and cholesterol efflux^{31A}. The apparent unfavorable effect of the usf1s2 risk allele on APOE expression shown here, follows fittingly from our earlier findings of the association of USF1 with FHCL and component traits^{6A}.

The correlation of the ACACA expression with insulin levels replicated the earlier findings,^{18A} but additionally revealed an important difference in the extent of this correlation between the two USF1 allelic haplotypes. The correlation was especially strong within the protective haplotype group. This differential transcriptional response to insulin is very interesting, given the known role of USF1 in mediating the response of metabolic genes to changes in insulin and glucose levels^{16A}. ACACA occupies a key position in overall lipid metabolism as the enzyme catalyzing the rate-limiting step in the biosynthesis of long-chain fatty acids^{32A}. These findings suggest a role for USF1 in the complex molecular pathway resulting in a well established insulin resistance in tissues of patients with FCHL and the metabolic syndrome.

An investigation of the USF1 regional genes did not show any influence of the usf1s2 alleles over their expression, suggesting that the effects are contained to the USF1 gene. However, a small unknown EST (AW995043) immediately 3′ of F11R was expressed differently between the groups carrying different alleles at usf1s2. ESTs usually represent fragments of transcribed genes, but as AW995043 is transcribed from the opposite strand compared to F11R and has no overlap with any known splice variant, it doesn't seem to be a part of it. The differential expression of this EST may be an anomaly, or it could represent a small regulatory RNA molecule with an as of yet unknown function. In a preferred embodiment, the nucleic acid molecule of the present invention is genomic DNA. This preferred embodiment of the invention reflects the fact that usually the analysis would be carried out on the basis of genomic DNA from body fluid, cells or tissue isolated from the person under investigation. In a further preferred embodiment of the nucleic acid molecule of the invention said genomic DNA is part of a gene. In accordance with the invention, it is preferred that at least intron 7 of the USF1 gene harboring SNP1 in position 3966 and/or exon 11 of the USF1 gene harboring SNP2 in position 5205 relative to the USF1 gene is analyzed. It is a central aspect of the present invention that a guanine residue in position 3966 of the USF1 gene indicates the presence of a disease-associated allele, whereas an adenine residue in the same position of the USF1 gene is indicative for the healthy allele. Likewise, a cytosine residue in position 5205 of the USF1 gene indicates the presence of a disease-associated allele, whereas a thymine residue is indicative for the healthy allele.

The present invention also relates to a fragment of the nucleic acid molecule the present invention having at least 20 nucleotides wherein said fragment comprises nucleotide position 3966 and/or position 5205 of SEQ ID NO:1. The fragment, of the invention may be of natural as well as of (semi)synthetic origin. Thus, the fragment may, for example, be a nucleic acid molecule that has been synthesized according to conventional protocols of organic chemistry. Importantly, the nucleic acid fragment of the invention comprises nucleotide position 3966 in intron 7 of the USF1 gene or nucleotide position 5205 in exon 11 of the USF1 gene. In these positions, the fragment may have either the wild-type nucleotide or the nucleotide contributing to or indicative of hyperlipidemia and/or dyslipidemia and/or defective carbohydrate metabolism (also referred to as the “mutant” or “disease-associated” sequence). Consequently, the fragment of the invention may be used, for example, in assays differentiating between the wild-type and the mutant sequence.

It is further preferred that the fragment of the invention consists of at least 17 nucleotides, more preferred at least 20 nucleotides, and most preferred at least 25 nucleotides such as 30 nucleotides. Preferably, however, the fragment is of up to 100 bp, up to 200 bp, up to 300 bp, up to 400 bp, up to 500 bp, up to 600 bp, up to 700 bp, up to 800 bp, up to 900 bp or up to 1000 bp in length.

Furthermore, the invention relates to a nucleic acid molecule which is complementary to the nucleic acid molecule of the present invention and which has a length of at least 17 or of at least 20 nucleotides. Preferably, however, complementary nucleic acid molecule is of up to 100 bp, up to 200 bp, up to 300 bp, up to 400 bp, up to 500 bp, up to 600 bp, up to 700 bp, up to 800 bp, up to 900 bp or up to 1000 bp in length.

This embodiment of the invention comprising at least 15 or at least 20 nucleotides and covering at least position 3966 or position 5205 of the USF1 gene is particularly useful in the analysis of the genetic setup in the recited positions in hybridization assays. Thus, for example, a 15 mer exactly complementary either to the wild-type sequence or to the variants contributing to or indicative of hyperlipidemia and/or dyslipidemia and/or defective carbohydrate metabolism may be used to differentiate between the polymorphic variants. This is because a nucleic acid molecule labeled with a detectable label not exactly complementary to the DNA in the analyzed sample will not give rise to a detectable signal, if appropriate hybridization and washing conditions are chosen.

In this regard, it is important to note that the nucleic acid molecule of the invention, the fragment thereof as well as the complementary nucleic acid molecule may be detectably labeled. Detectable labels include radioactive labels such as ³H, or ³²P or fluorescent labels. Labeling of nucleic acids is well understood in the art and described, for example, in Sambrook et al., “Molecular Cloning, A Laboratory Manual”; ISBN: 0879695765, CSH Press, Cold Spring Harbor, 2001.

Hybridisation is preferably performed under stringent or highly stringent conditions. “Stringent or highly stringent conditions” of hybridization are well known to or can be established by the person skilled in the art according to conventional protocols. Appropriate stringent conditions for each sequence may be established on the basis of well-known parameters such as temperature, composition of the nucleic acid molecules, salt conditions etc.: see, for example, Sambrook et al., “Molecular Cloning, A Laboratory Manual”; ISBN: 0879695765, CSH Press, Cold Spring Harbor, 2001 and earlier edition Sambrook et al., “Molecular Cloning, A Laboratory Manual”; CSH Press, Cold Spring Harbor, 1989 or Higgins and Hames (eds.), “Nucleic acid hybridization, a practical approach”, IRL Press, Oxford 1985 (reference 54), see in particular the chapter “Hybridization Strategy” by Britten & Davidson, 3 to 15. Typical (highly stringent) conditions comprise hybridization at 65° C. in 0.5×SSC and 0.1% SDS or hybridization at 42° C. in 50% formamide, 4×SSC and 0.1% SDS. Hybridization is usually followed by washing to remove unspecific signal. Washing conditions include conditions such as 65° C., 0.2×SSC and 0.1% SDS or 2×SSC and 0.1% SDS or 0.3×SSC and 0.1% SDS at 25° C.-65° C. Hybridisation may also be performed under conditions of lower stringency. The parameters of such hybridization conditions are described in Sambrook et al., “Molecular Cloning, A Laboratory Manual”; ISBN: 0879695765, CSH Press, Cold Spring Harbor, 2001 in more detail. A non-limiting, example of low stringency hybridization conditions are hybridization in 35% formamide, 5.times.SSC, 50 mM Tris-HCl (pH 7.5), 5 mM EDTA, 0.02% PVP, 0.02%/o Ficoll, 0.2% BSA, 100 mg/ml denatured salmon sperm DNA, 10% (wt/vol) dextran sulfate at 40.degree.C., followed by one or more washes in 2.times.SSC, 25 mM Tris-HCl (pH 7.4), 5 mM EDTA, and 0.1% SDS at 50.degree.C. Other conditions of low stringency that may be used are well known in the art (e.g., as employed for cross-species hybridizations). See, e.g., Ausubel, et al. (eds.), 1993; CURRENT PROTOCOLS IN MOLECULAR BIOLOGY, John Wiley & Sons. NY, and Kriegler, 1990, GENE TRANSFER AND EXPRESSION, A LABORATORY MANUAL, Stockton Press, NY; Shilo and Weinberg, 1981, Proc Natl Acad Sci USA 78: 6789-6792.

In addition, the invention relates to a vector comprising the nucleic acid molecule as described herein above. The vectors may particularly be plasmids, cosmids, viruses or bacteriophages used conventionally in genetic engineering that comprise the nucleic acid molecule of the invention. Preferably, said vector is an expression vector and/or a gene transfer or targeting vector. Expression vectors derived from viruses such as retroviruses, vaccinia virus, adeno-associated virus, herpes viruses, or bovine papilloma virus, may be used for delivery of the nucleic acid molecule of the invention into targeted cell population. Methods which are well known to those skilled in the art can be used to construct recombinant viral vectors; see, for example, the techniques described in Sambrook et al., loc. cit. and Ausubel et al., Current Protocols in Molecular Biology, Green Publishing Associates and Wiley Interscience, N.Y. (2001). Alternatively, the nucleic acid molecules and vectors of the invention can be reconstituted into liposomes for delivery to target cells. The vectors containing the nucleic acid molecules of the invention can be transferred into the host cell by well-known methods, which vary depending on the type of cellular host. For example, calcium chloride transfection is commonly utilized for prokaryotic cells, whereas, e.g., calcium phosphate or DEAE-Dextran mediated transfection or electroporation may be used for other cellular hosts; see Sambrook, supra.

Such vectors may comprise further genes such as marker genes which allow for the selection of said vector in a suitable host cell and under suitable conditions. Preferably, the nucleic acid molecule of the invention is operatively linked to expression control sequences allowing expression in prokaryotic or eukaryotic cells. Expression of said polynucleotide comprises transcription of the polynucleotide into a translatable mRNA. Regulatory elements ensuring expression in eukaryotic cells, preferably mammalian cells, are well known to those skilled in the art. They usually comprise regulatory sequences ensuring initiation of transcription and, optionally, a poly-A signal ensuring termination of transcription and stabilization of the transcript, and/or an intron further enhancing expression of said polynucleotide. Additional regulatory elements may include transcriptional as well as translational enhancers, and/or naturally-associated or heterologous promoter regions. Possible regulatory elements permitting expression in prokaryotic host cells comprise, e.g., the PL, lac, trp or tac promoter in E. coli, and examples for regulatory elements permitting expression in eukaryotic host cells are the AOX1 or GAL1 promoter in yeast or the CMV-, SV40-, RSV-promoter (Rous sarcoma virus), CMV-enhancer, SV40-enhancer or a globin intron in mammalian and other animal cells. Beside elements which are responsible for the initiation of transcription such regulatory elements may also comprise transcription termination signals, such as the SV40-poly-A site or the tk-poly-A site, downstream of the polynucleotide. Optionally, the heterologous sequence can encode a fusion protein including an C- or N-terminal identification peptide imparting desired characteristics, e.g., stabilization or simplified purification of expressed recombinant product. In this context, suitable expression vectors are known in the art such as Okayama-Berg cDNA expression vector pcDV1 (Pharmacia), pCDM8, pRc/CMV, pcDNA1, pcDNA3, the Echo™ Cloning System (Invitrogen), pSPORT1 (GIBCO BRL) or pRevTet-On/pRevTet-Off or pCI (Promega).

Preferably, the expression control sequences will be eukaryotic promoter systems in vectors capable of transforming or transfecting eukaryotic host cells, but control sequences for prokaryotic hosts may also be used.

As mentioned above, the vector of the present invention may also be a gene transfer or targeting vector. Gene therapy, which is based on introducing therapeutic genes into cells by ex-vivo or in-vivo techniques is one of the most important applications of gene transfer. Suitable vectors and methods for in-vitro or in-vivo gene therapy are described in the literature and are known to the person skilled in the art; see, e.g., Giordano, Nature Medicine 2 (1996), 534-539; Schaper, Circ. Res. 79 (1996), 911-919; Anderson, Science 256 (1992), 808-813; Isner, Lancet 348 (1996), 370-374; Muhlhauser, Circ. Res. 77 (1.995), 1077-1086; Wang, Nature Medicine 2 (1996), 714-716; WO94/29469; WO 97/00957, Schaper, Current Opinion in Biotechnology 7 (1996), 635-640, or Kay et al. (2001) Nature Medicine, 7, 33-40) and references cited therein. The polynucleotides and vectors of the invention may be designed for direct introduction or for introduction via liposomes, or viral vectors (e.g. adenoviral, retroviral) into the cell. Preferably, said cell is a germ line cell, embryonic cell, or egg cell or derived therefrom, most preferably said cell is a stem cell. Gene therapy is envisaged with the wild-type nucleic acid molecule only.

The invention also relates to a primer or primer pair, wherein the primer or primer pair hybridizes under stringent conditions to the nucleic acid molecule of the present invention comprising nucleotide positions 3966 and/or 5205 SEQ ID NO:1 or to the complementary strand thereof. In a preferred embodiment, said primer has an adenine or a guanine residue in the position corresponding to position 3966 of the USF1 sequence. In another preferred embodiment, said primer has a cytosine or a thymine residue in the position corresponding to position 5205 of the USF1 sequence. The primer may bind to the coding (+) strand or to the non-coding (−) strand of the DNA double strand.

Preferably, the primers of the invention have a length of at least 14 nucleotides such as 17, 20 or 21 nucleotides. The fact that in one embodiment the target sequence of the primer is located 3′ to the SNP is to ensure that the primer is actually useful for sequence analysis, i.e. that the elongated primer sequence actually contains the SNP. When a PCR reaction is performed, for example, usually two primers are involved, wherein one primer binds 3′ of the SNP on the +strand and the other primer binds 3′ of the SNP on the − strand.

In one embodiment, the primer actually binds to the position of the SNP. As a consequence, when binding is performed under stringent conditions, such a primer is useful to distinguish between different polymorphic variants as binding only occurs if the sequences of the primer and the target have full complementarity. It is further preferred that the primers have a maximum length of 24 nucleotides. However, in particular cases it may be preferable to use primers with a maximum length of 30 of 35 nucleotides. Hybridization or lack of hybridization of a primer under appropriate conditions to a genome sequence comprising either position 3966 or position 5205 coupled with an appropriate detection method such as an elongation reaction or an amplification reaction may be used to differentiate between the polymorphic variants and then draw conclusions with regard to, e.g., the predisposition of the person under investigation hyperlipidemia and/or dyslipidemia and/or defective carbohydrate metabolism. The present invention envisages two types of primers/primer pairs. One type hybridizes to a sequence comprising the mutant, i.e. disease-associated sequence. In other terms. One nucleotide of the primer pairs with the guanine residue in position 3966 (or the cytosine residue of the complementary strand) or with the thymine residue in position 5205 (or the adenine residue in the complementary strand). The other type of primer is exactly complementary to a sequence of wild-type. Since hybridization conditions would preferably be chosen to be stringent enough, contacting of e.g. a primer exactly complementary to the mutant sequence with a wild-type allele would not result in efficient hybridization due to the mismatch formation. After washing, no signal would be detected due to the removal of the primer.

Additionally, the invention relates to a non-human host transformed with the vector of the invention as described herein above. The host may either carry the mutant or the wild-type sequence. Upon breeding etc. the host may be heterozygous or homozygous for one or both SNPs.

The host of the invention may carry the vector of the invention either transiently or stably integrated into the genome. Methods for generating the non-human host of the invention are well known in the art. For example, conventional transfection protocols described in Sambrook et al., loc. cit., may be employed to generate transformed bacteria (such as E. coli) or transformed yeasts. The non-human host of the invention may be used, for example, to elucidate the onset of hyperlipidemia and/or dyslipidemia and/or defective carbohydrate metabolism.

In a preferred embodiment of the invention the non-human host is a bacterium, a yeast cell, an insect cell, a fungal cell, a mammalian cell, a plant cell, a transgenic animal or a transgenic plant.

Whereas E. coli is a preferred bacterium, preferred yeast cells are S. cerevisiae or Pichia pastoris cells. Preferred fungal cells are Aspergillus cells and preferred insect cells include Spodoptera frugiperda cells. Preferred mammalian cells are CHO cells, colon carcinoma and hepatoma cell lines showing expression of the USF1 transcription factor. However, also cell lines with very low expression of USF1, including HeLa cells and the like or fibroblasts, might be particularly useful for specific experiments.

A method for the production of a transgenic non-human animal, for example transgenic mouse, comprises introduction of the aforementioned polynucleotide or targeting vector into a germ cell, an embryonic cell, stem cell or an egg or a cell derived therefrom. The non-human animal can be used in accordance with a screening method of the invention described herein. Production of transgenic embryos and screening of those can be performed, e.g., as described by A. L. Joyner Ed., Gene Targeting, A Practical Approach (1993), Oxford University Press. The DNA of the embryonal membranes of embryos can be analyzed using, e.g., Southern blots with an appropriate complementary nucleic acid molecule; see supra. A general method for making transgenic non-human animals is described in the art, see for example WO 94/24274. For making transgenic non-human organisms (which include homologously targeted non-human animals), embryonal stem cells (ES cells) are preferred. Murine ES cells, such as AB-1 line grown on mitotically inactive SNL76/7 cell feeder layers (McMahon and Bradley, Cell 62:1073-1085 (1990)) essentially as described (Robertson, E. J. (1987) in Teratocarcinomas and Embryonic Stem Cells: A Practical Approach. E. J. Robertson, ed. (Oxford: IRL Press), p. 71-112) may be used for homologous gene targeting. Other suitable ES lines include, but are not limited to, the E14 line (Hooper et al., Nature 326:292-295 (1987)), the D3 line (Doetschman et al., J. Embryol. Exp. Morph. 87:27-45 (1985)), the CCE line (Robertson et al., Nature 323:445-448 (1986)), the AK-7 line (Zhuang et al., Cell 77:875-884 (1994)). The success of generating a mouse line from ES cells bearing a specific targeted mutation depends on the pluripotence of the ES cells (i.e., their ability, once injected into a host developing embryo, such as a blastocyst or morula, to participate in embryogenesis and contribute to the germ cells of the resulting animal). The blastocysts containing the injected ES cells are allowed to develop in the uteri of pseudopregnant nonhuman females and are born as chimeric mice. The resultant transgenic mice are chimeric for cells having the desired nucleic acid molecule are backcrossed and screened for the presence of the correctly targeted transgene (s) by PCR or Southern blot analysis on tail biopsy DNA of offspring so as to identify transgenic mice heterozygous for the nucleic acid molecule of the invention.

The transgenic non-human animals may, for example, be transgenic mice, rats, hamsters, dogs, monkeys (apes), rabbits, pigs, or cows. Preferably, said transgenic non-human animal is a mouse. The transgenic animals of the invention are, inter alia, useful to study the phenotypic expression/outcome of the nucleic acids and vectors of the present invention. Furthermore, the transgenic animals of the present invention are useful to study the developmental expression of the USF1 gene and of its role for onset of hyperlipidemia and/or dyslipidemia and/or defective carbohydrate metabolism, for example in the rodent intestine. It is furthermore envisaged, that the non-human transgenic animals of the invention can be employed to test for therapeutic agents/compositions or other possible therapies which are useful to hyperlipidemia and/or dyslipidemia and/or defective carbohydrate metabolism.

The present invention also relates to a pharmaceutical composition comprising USF1 or a fragment thereof, a nucleic acid molecule encoding USF1 or a fragment thereof, or an antibody specific for USF1.

The components of the pharmaceutical composition of the invention may be combined with a pharmaceutically acceptable carrier and/or diluent and/or excipient. Preferably, USF1 refers to any USF1 being capable of alleviating the disease symptoms. Generally, USF1 will be of wild-type. However, in particular cases it might also be useful to administer mutated USF1 having one or more point mutations, insertions, deletions and the like and showing increased or decreased function or activity. Also encompassed by the present invention are chemically modified molecules which improve uptake or stability of a polypeptide.

Examples of suitable pharmaceutical carriers are well known in the art and include phosphate buffered saline solutions, water, emulsions, such as oil/water emulsions, various types of wetting agents, sterile solutions etc. Compositions comprising such carriers can be formulated by well known conventional methods. These pharmaceutical compositions can be administered to the subject at a suitable dose. Administration of the suitable compositions may be effected by different ways, e.g., by intravenous, intraperitoneal, subcutaneous, intramuscular, topical, intradermal, intranasal or intrabronchial administration. The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. A typical dose can be, for example, in the range of 0.001 to 1000 μg of nucleic acid for expression or for inhibition of expression; however, doses below or above this exemplary range are envisioned, especially considering the aforementioned factors. Dosages will vary but a preferred dosage for intravenous administration of DNA is from approximately 10⁶ to 10¹² copies of the DNA molecule. Progress can be monitored by periodic assessment. The compositions of the invention may be administered locally or systemically. Administration will generally be parenterally, e.g., intravenously; DNA may also be administered directly to the target site, e.g., by biolistic delivery to an internal or external target site or by catheter to a site in an artery. Preparations for parenteral administration include sterile aqueous or non-aqueous solutions, suspensions, and emulsions. Examples of non-aqueous solvents are propylene glycol, polyethylene glycol, vegetable oils such as olive-oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Additionally, the invention relates to a diagnostic composition comprising a nucleic acid molecule encoding USF1 or a fragment thereof, the nucleic acid molecule as described herein above, the vector as described herein above, the primer or primer pair as described herein above or an antibody specific for USF1.

The diagnostic composition is useful for assessing the genetic status of a person with respect to his or her predisposition to develop hyperlipidemia and/or dyslipidemia and/or defective carbohydrate metabolism or with regard to the diagnosis of the acute condition. The various possible components of the diagnostic composition may be packaged in one or more vials, in a solvent or otherwise such as in lyophilized form. If dissolved in a solvent, the diagnostic composition is preferably cooled to at least +8° C. to +4° C. Freezing may be preferred in other instances.

The present invention also relates to a method for testing for the presence or predisposition of hyperlipidemia and/or dyslipidemia and/or defective carbohydrate metabolism, comprising analyzing a sample obtained from a prospective patient or from a person suspected of carrying such a predisposition for the presence of a wild-type or variant allele of the USF1 gene. Preferably, said variant comprises an SNP at position 3966 and/or at position 5205 of the USF1 gene in a homozygous or heterozygous state. In varying embodiments, it may be tested either for the presence of the wild-type sequence(s) or of the mutant sequence(s). It is in accordance with the present invention that a guanine residue in position 3966 of the USF1 gene indicates the presence of a disease-associated allele, whereas an adenine residue in the same position of the USF1 gene is indicative for the healthy allele. Likewise, a cytosine residue in position 5205 of the USF1 gene indicates the presence of a disease-associated allele, whereas a thymine residue is indicative for the healthy allele.

The method of the invention is useful for detecting the genetic set-up of said person/patient and drawing appropriate conclusions whether a condition from which said patient suffers is hyperlipidemia and/or dyslipidemia and/or defective carbohydrate metabolism. Alternatively, it may be assessed whether a person not suffering from a condition carries a predisposition to hyperlipidemia and/or dyslipidemia and/or defective carbohydrate metabolism. With regard to position 5205 in exon 11 of the USF1 gene, only if cytosine is found in a homozygous or heterozygous state, a condition would be diagnosed as hyperlipidemia and/or dyslipidemia and/or defective carbohydrate metabolism or a corresponding predisposition would be manifest. On the other hand, if thymine is found in a homozygous state, then it may be concluded that a condition from which a patient suffers is not related to hyperlipidemia or dyslipidemia and/or defective carbohydrate metabolism and further, that the patient does not carry a predisposition to develop this condition. The situation is similar and essentially the same conclusions apply for the analysis of the SNP in position 3966: With regard to position 3966 in intron 7 of the USF1 gene, only if guanine is found in a homozygous or heterozygous state, a condition would be diagnosed as hyperlipidemia and/or dyslipidemia and/or defective carbohydrate metabolism or a corresponding predisposition would be manifest. On the other hand, if an adenine is found in a homozygous state, then it may be concluded that a condition from which a patient suffers is not related to hyperlipidemia or dyslipidemia and/or defective carbohydrate metabolism and further, that the patient does not carry a predisposition to develop this condition.

In a preferred embodiment of the method of the invention said testing comprises hybridizing the complementary nucleic acid molecule as described herein above which is complementary to the nucleic acid molecule contributing to or indicative of hyperlipidemia and/or dyslipidemia and/or defective carbohydrate metabolism or the nucleic acid molecule as described herein above which is complementary to the wild-type sequence as a probe under (highly) stringent conditions to nucleic acid molecules comprised in said sample and detecting said hybridization, wherein said complementary nucleic acid molecule comprises the sequence position containing the SNP.

Again, depending on the nucleic acid probe used, either wild-type or mutant sequences (i.e. sequences contributing to or indicative of hyperlipidemia and/or dyslipidemia and/or defective carbohydrate metabolism) would be detected. It is understood that hybridization conditions would be chosen such that a nucleic acid molecule complementary to wild-type sequences would not or essentially not hybridize to the mutant sequence. Similarly, a nucleic acid molecule complementary to the mutant sequence would not or would not essentially not hybridize to the wild-type sequence. In order to differentiate between results obtained from homozygous and heterozygous genotypes in the hybridization methods of the invention, one can for example monitor/detect the strength/intensity of the respective detection signal after the hybridization. To differentiate between wild-type homozygous, heterozygous and/or mutant homozygous alleles in the hybridization methods of the invention, internal control samples of the corresponding genotypes will be included in the analysis.

In a further preferred embodiment, the method of the invention further comprises digesting the product of said hybridization with a restriction endonuclease or subjecting the product of said hybridization to digestion with a restriction endonuclease and analyzing the product of said digestion.

This preferred embodiment of the invention allows by convenient means, the differentiation between an effective hybridization and a non-effective hybridization. For example, if the DNA sequence adjacent to position 3966 or position 5205 comprises an endonuclease restriction site, the hybridized product will be cleavable by an appropriate restriction enzyme upon an effective hybridization whereas a lack of hybridization will yield no double-stranded product or will not comprise the recognizable restriction site and, accordingly, will not be cleaved. Suitable restriction enzymes may be found, for example, by the use of the program Webcutter. The analysis of the digestion product can be effected by conventional means, such as by gel electrophoresis which may be optionally combined by the staining of the nucleic acid with, for example, ethidium bromide. Combinations with further techniques such as Southern blotting are also envisaged.

Detection of said hybridization may be effected, for example, by an anti-DNA double-strand antibody or by employing a labeled oligonucleotide. Conveniently, the method of the invention is employed together with blotting techniques such as Southern or Northern blotting and related techniques. Labeling may be effected, for example, by standard protocols and includes labeling with radioactive markers, fluorescent, phosphorescent, chemiluminescent, enzymatic labels, etc. The label can be located at the 5′ and/or 3′ end of the nucleic acid molecule or be located at an internal position. Preferred labels include, but are not limited to, fluorochromes, e.g. Carboxyfluorescein (FAM) and 6-carboxy-X-rhodamine (ROX), fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), radioactive labels, e.g. ³²P, ³⁵S, ³H; etc. The label may also be a two stage system, where the probe is conjugated to biotin, haptens, etc. having a high affinity binding partner, e.g. avidin, specific antibodies, etc., where the binding partner is conjugated to a detectable label.

In accordance with the above, in another preferred embodiment of the method of the invention said probe is detectably labeled, e.g. by the methods and with the labels described herein above.

In yet another preferred embodiment of the method of the invention said testing comprises determining the nucleic acid sequence of at least a portion of the nucleic acid molecule as described herein above, said portion comprising the position of the SNP. Determination of the nucleic acid molecule may be effected in accordance with one of the conventional protocols such as the Sanger or Maxam/Gilbert protocols (see Sambrook et al., loc. cit., for further guidance).

In a further preferred embodiment of the method of the invention the determination of the nucleic acid sequence is effected by solid-phase minisequencing. Solid-phase minisequencing is based on quantitative analysis of the wild type and mutant nucleotide in a solution. First, the genomic region containing the mutation is amplified by PCR with one biotinylated and non-biotinylated primer where the biotinylated primer is attached to a streptavidin (SA) coated plate. The PCR-product is denatured to a single stranded form to allow a minisequencing primer to bind to this strand just before the site of the mutation. The tritium (H3) or fluorescence labeled mutated and wild type nucleotides together with nonlabeled dNTPs are added to the minisequencing reaction and sequenced using Taq-polymerase. The result is based on the amount of wild type and mutant nucleotides in the reaction measured by beta counter or fluorometer and expressed as an R-ratio. See also Syvänen A C, Sajantila A, Lukka M. Am J Hum Genet 1993: 52, 46-59 and Suomalainen A and Syvanen AC. Methods Mol Biol 1996; 65:73-79.

A preferred embodiment of the method of the invention further comprises, prior to determining said nucleic acid sequence, amplification of at least said portion of said nucleic acid molecule. Preferably, amplification is effected by polymerase chain reaction (PCR). Other amplification methods such as ligase chain reaction may also be employed.

In a preferred embodiment of the method of the invention said testing comprises carrying out an amplification reaction wherein at least one of the primers employed in said amplification reaction is the primer as described herein above or belongs to the primer pair as described herein above, comprising assaying for an amplification product. In this embodiment and depending on the information the investigator/physician wishes to obtain, primers hybridizing either to the wild-type or mutant sequences may be employed. In a particularly preferred embodiment, at least one of the primers will actually bind to the position of the SNP. As a consequence, when binding is performed under stringent conditions, such a primer is useful to distinguish between different polymorphic variants as binding only occurs if the sequences of the primer and the target have full complementarity.

The method of the invention will result in an amplification of only the target sequence, if said target sequence carries a sequence exactly complementary to the primer used for hybridization. This is because the oligonucleotide primer will under preferably (highly) stringent hybridization conditions not hybridize to the wildtype/mutant sequence—depending which type of primer is used—(with the consequence that no amplification product is obtained) but only to the exactly matching sequence. Naturally, combinations of primer pairs hybridizing to both SNPs may be used. In this case, the analysis of the amplification products expected (which may be no, one, two, three or four amplification product(s) if the second, non-differentiating primer is the same for each locus) will provide information on the genetic status of both positions 3966 and 5205.

In a preferred embodiment of the method of the invention said amplification is effected by or said amplification is the polymerase chain reaction (PCR). The PCR is well established in the art. Typical conditions to be used in accordance with the present invention include for example a total of 35 cycles in a total of 50 μl volume exemplified with a denaturation step at 93° C. for 3 minutes; an annealing step at 55° C. for 30 seconds; an extension step at 72° C. for 75 seconds and a final extension step at 72° C. for 10 minutes.

The present invention further relates to a method for testing for the presence or predisposition of hyperlipidemia and/or dyslipidemia and/or defective carbohydrate metabolism comprising assaying a sample obtained from a human for the amount of (a) USF1, (b) ABCA1, (c) angiotensinogen or (d) apolipoprotein E contained in said sample. The amount of USF1 can be determined by any suitable method. Preferably, the amount of USF1 is determined by contacting the sample, i.e. USF1 contained in the sample, with an antibody or aptamer or a derivative thereof, which is specific for (a) USF1, (b) ABCA1, (c) angiotensinogen or (d) apolipoprotein E. For example, the sample containing USF1 may be analyzed in a Western blot or in a RIA assay. In this context a weaker staining for the presence of the antigen of the invention compared to homozygous wild-type control samples (comprising two persistent alleles) is indicative for the heterozygous wild type (one persistent allele and one disease-associated allele), whereas for the homozygous disease state no staining or a reduced staining is expected if the appropriate antibody is used. Preferably, the method of the invention is performed in the presence of control samples corresponding to all three possible allelic combinations as internal controls. Testing may be carried out with an antibody or aptamer etc. specific for the wild-type or specific for the mutant sequence. Testing for binding may, again, involve the employment of standard techniques such as ELISAs; see, for example, Harlow and Lane⁵³, loc. cit. The term “antibody” as used throughout the invention refers to monoclonal antibodies, polygonal antibodies, single chain antibodies, or a fragment thereof. Preferably the antibody is specific for USF1 or for wild-type or disease-associated USF1. The antibodies may be bispecific antibodies, humanized antibodies, synthetic antibodies, antibody fragments, such as Fab, a F(ab₂)′, Fv or scFv fragments etc., or a chemically modified derivative of any of these (all comprised by the term “antibody”). Monoclonal antibodies can be prepared, for example, by the techniques as originally described in Köhler and Milstein, Nature 256 (1975), 495, and Galfré, Meth. Enzymol. 73 (1981), 3, which comprise the fusion of mouse myeloma cells to spleen cells derived from immunized mammals with modifications developed by the art. Antibodies may be labelled by using any of the labels described in the present invention.

In a preferred embodiment of the method of the invention said antibody or aptamer is detectably labeled. Whereas the aptamers are preferably radioactively labeled with ³H or ³²P or with a fluorescent marker, the antibody may either be labeled in a corresponding manner (with ¹³¹I as the preferred radioactive label) or be labeled with a tag such as His-tag, FLAG-tag or myc-tag.

In a further preferred embodiment of the method of the invention the test is an immuno-assay.

The present invention also relates to a method for testing for the presence or predisposition of hyperlipidemia and/or dyslipidemia and/or defective carbohydrate metabolism comprising assaying a sample obtained from a human for the amount of RNA encoding (a) ABCA1, (b) angiotensinogen or (c) apolipoprotein E contained in said sample. Testing may be performed by any of the methods known to the skilled person, such as northern blot analysis or by the methods described herein.

In another preferred embodiment of the method of the invention said sample is blood, serum, plasma, fetal tissue, saliva, urine, mucosal tissue, mucus, vaginal tissue, fetal tissue obtained from the vagina, skin, hair, hair follicle or another human tissue.

In an additional preferred embodiment of the method of the invention said nucleic acid molecule from said sample is fixed to a solid support.

Fixation of the nucleic acid molecule to a solid support will allow an easy handling of the test assay and furthermore, at least some solid supports such as chips, silica wafers or microtiter plates allow for the simultaneous analysis of larger numbers of samples. Ideally, the solid support allows for an automated testing employing, for example, roboting devices.

In a particularly preferred embodiment of the method of the invention said solid support is a chip, a silica wafer, a bead or a microtiter plate.

The methods of the present invention may be performed ex vivo, in vitro or in vivo.

The present invention also relates to the use of a nucleic acid molecule encoding USF1, the nucleic acid molecule as described herein above, or of USF1 polypeptide for the analysis of the presence or predisposition of hyperlipidemia, dyslipidemia and/or defective carbohydrate metabolism. The nucleic acid molecule simultaneously allows for the analysis of the absence of the condition or the predisposition to the condition, as has been described in detail herein above. In particular cases, it may be possible to use USF1 polypeptides for testing. This may be, for example, in cases when expression of USF1 results in an autoimmune response against USF1. In such cases it will be possible, by using USF1 polypeptides, to monitor patients by detecting antibodies directed against USF1. Such assays can, for example, be based on the western blotting technique or by performing (radio)immunoprecipitations.

In addition, the present invention relates to the use of USF1 or a fragment thereof, a nucleic acid molecule encoding USF1 and/or comprising at least the wild-type sequence of intron 7 and/or exon 11 of USF1, for the preparation of a pharmaceutical composition for the treatment of hyperlipidemias and/or dyslipidemias, including familial combined hyperlipidemia (FCHL), hypercholesterolemia, hypertriglyceridemia, hypoalphalipoproteinemia, hyperapobetalipoproteinemia (hyperapoB) and/or familial dyslipidemic hypertension (FDH), coronary heart disease, type II diabetes, atherosclerosis or metabolic syndrome. Any of the diseases mentioned in the present invention can be treated by administering to a patient USF1 in an amount and quality sufficient to ameliorate the symptoms of the disease. If for example the disease symptoms are created by a reduced amount of USF1 in the patient, administration of USF1 to the patient will compensate for the reduced USF1 of the patient. USF1 may be provided to the patient as such, i.e. as the polypeptide. Alternatively, a nucleic acid molecule encoding USF1 can be administered. Preferably, USF1 is a full length wild-type polyprotein. However, in particular cases it might also be useful to administer mutated USF1 having one or more point mutations, insertions, deletions and the like and showing increased or decreased function or activity. Also encompassed by the present invention are chemically modified molecules which improve uptake or stability of a polypeptide. Gene therapy approaches have been discussed herein above in connection with the vector of the invention and equally apply here. It is of note that in accordance with this invention, also fragments of the nucleic acid molecules as defined herein above may be employed in gene therapy approaches. Said fragments comprise the nucleotide at position 3966 as or position 5205 of the USF1 gene. Preferably, said fragments comprise at least 200, at least 250, at least 300, at least 400 and most preferably at least 500 nucleotides. In a preferred embodiment of the use of the invention said gene therapy treats or prevents hyperlipidemia and/or dyslipidemia and/or defective carbohydrate metabolism.

The present invention relates to a kit comprising the nucleic acid molecule, the primer or primer pair and/or the vector of the present invention in one or more containers.

The present invention also relates to the use of an inhibitor of expression of USF1, wherein said inhibitor is (a) an siRNA or antisense RNA molecule comprising a nucleotide sequence complementary to the transcribed region of the USF1 gene or (b) of an antibody, aptamer or small inhibitory molecule specific for USF1 gene, for the preparation of a pharmaceutical composition for the treatment of hyperlipidemias and/or dyslipidemias including familial combined hyperlipidemia (FCHL), hypercholesterolemia, hypertriglyceridemia, hypoalphalipoproteinemia, hyperapobetalipoproteinemia (hyperapoB), familial dyslipidemic hypertension (FDH), metabolic syndrome, type 2 diabetes mellitus, coronary heart disease, atherosclerosis or hypertension.

The inhibitor molecules disclosed in the present invention can be used in vivo or in vitro. In one embodiment of the present invention, the inhibitory RNA molecules, aptamers and antibodies are expressed from an expression cassette. This expression cassette can e.g. be used to generate stable cell lines expressing the siRNA disclosed herein. Stable cell lines may be based e.g. on stem cells obtainable from a patient in need of treatment of the diseases mentioned in the present invention. These stable cell lines may be re-introduced into the patient. In another embodiment of the present invention, the siRNA is expressed from a viral vector. Expression of siRNA will result in a downregulation of specific target genes.

As used herein, the term “siRNA” means “short interfering RNA”. In RNA interference, small interfering RNAs (siRNA) bind the targeted mRNA in a sequence-specific manner, facilitating its degradation and thus preventing translation of the encoded protein. Transfection of cells with siRNAs can be achieved, for example, by using lipophilic agents (among them Oligofectamine™ and Transit-TKO™) and also by electroporation.

Methods for the stable expression of small interfering RNA or short hairpin RNA in mammalian, also in human cells are known to the person skilled in the art and are described, for example, by Paul et al. 2002 (Nature Biotechnology 20: 505-508), Brummelkamp et al. 2002 (Science 296: 550-553), Sui et al. 2002 (Proc. Natl. Acad. Sci. U.S.A. 99: 5515-5520), Yu et al. 2002 (Proc. Natl. Acad. Sci. U.S.A. 99: 6047-6052), Lee et al. 2002 (Nature Biotechnology 20: 500-505), Xia et al. 2002 (Nature Biotechnology-20: 1006-1010). It has been shown by several studies that an RNAi approach is suitable for the development of a potential treatment of inherited diseases by designing a siRNA that specifically targets the disease-associated mutant allele, thereby selectively silencing expression from the mutant gene (Miller et al. 2003, Proc. Natl. Acad. Sci. U.S.A. 100: 7195-7200; Gonzalez-Alegre et al. 2003, Ann. Neurol. 53: 781-787).

The siRNA molecules are essentially double-stranded but may comprise 3′ or 5′ overhangs. They may also comprise sequences that are not identical or essentially identical with the target gene but these sequences must be located outside of the sequence of identity. The sequence of identity or substantial identity is at least 14 and more preferably at least 19 nucleotides long. It preferably does not exceed 23 nucleotides. Optionally, the siRNA comprises two regions of identity or substantial identity that are interspersed by a region of non-identity. The term “substantial identity” refers to a region that has one or two mismatches of the sense strand of the siRNA to the targeted mRNA or 10 to 15% over the total length of siRNA to the targeted mRNA mismatches within the region of identity. Said mismatches may be the result of a nucleotide substitution, addition, deletion or duplication etc. dsRNA longer than 23 but no longer than 40 bp may also contain three or four mismatches.

The interference of the siRNA with the targeted mRNA has the effect that transcription/translation is reduced by at least 50%, preferably at least 75%, more preferred at least 90%, still more preferred at least 95%, such as at least 98% and most preferred at least 99%.

The term “small molecule inhibitor” or “small molecular compound” refers to a compound having a relative molecular weight of not more than 1000 D and preferably of not more than 500 D. It can be of organic or inorganic nature. A large number of small molecule libraries, which are commercially available, are known in the art. Thus, for example, the small molecule inhibitor may be any of the compounds contained in such a library or a modified compound derived from a compound contained in such a library. Preferably, such an inhibitor binds to the targeted protein with sufficient specificity, wherein sufficient specificity means preferably a dissociation constant (Kd) of less than 500 nM, more preferable less than 200 nM, still more preferable less than 50 nM, even more preferable less than 10 nM and most preferable less than 1 nM.

The term “antisense nucleic acid molecule” refers to a nucleic acid molecule which can be used for controlling gene expression. The underlying technique, antisense technology, can be used to control gene expression through antisense DNA or RNA or through triple-helix formation. Antisense techniques are discussed, for example, in Okano, J. Neurochem. 56: 560 (1991); “Oligodeoxynucleotides as Antisense Inhibitors of Gene Expression.” CRC Press, Boca Raton, Fla. (1988), or in: Phillips M I (ed.), Antisense Technology, Methods in Enzymology, Vol. 313, Academic Press, San Diego (2000). Triple helix formation is discussed in, for instance, Lee et al., Nucleic Acids Research 6: 3073 (1979); Cooney et al., Science 241: 456 (1988); and Dervan et al., Science 251: 1360 (1991). The methods are based on binding of a target polynucleotide to a complementary DNA or RNA. For example, the 5′ coding portion of a polynucleotide that encodes USF1 may be used to design an antisense RNA oligonucleotide of from about 10 to 40 base pairs in length. A DNA oligonucleotide is designed to be complementary to a gene region involved in transcription thereby preventing transcription and the production of USF1. The antisense RNA oligonucleotide hybridizes to the mRNA in vivo and blocks translation of the mRNA molecule into USF1 protein.

The term “ribozyme” refers to RNA molecules with catalytic activity (see, e.g., Sarver et al, Science 247:1222-1225 (1990)); however, DNA catalysts (deoxyribozymes) are also known. Ribozymes and their potential for the development of new therapeutic tools are discussed, for example, by Steele et al. 2003 (Am. J. Pharmacogenomics 3: 131-144) and by Puerta-Fernandez et al. 2003 (FEMS Microbiology Reviews 27: 75-97). While ribozymes that cleave mRNA at site specific recognition sequences can be used to destroy USF1 mRNAs, the use of trans-acting hairpin or hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, Nature 334:585-591 (1988). There are numerous potential hammerhead ribozyme cleavage sites within the nucleotide sequence of the coagulation factor XII mRNA which will be apparent to the person skilled in the art. Preferably, the ribozyme is engineered so that the cleavage recognition site is located near the 5′ end of the mRNA; i.e., to increase efficiency and minimize the intracellular accumulation of non-functional mRNA transcripts. RNase P is another ribozyme approach used for the selective inhibition of pathogenic RNAs. Ribozymes may be composed of modified oligonucleotides (e.g. for improved stability, targeting, etc.) and should be delivered to cells which express USF1. DNA constructs encoding the ribozyme may be introduced into the cell by virtually any of the methods known to the skilled person. A preferred method of delivery involves using a DNA construct “encoding” the ribozyme under the control of a strong constitutive promoter, such as, for example, pol III or pol II promoter, so that transfected cells will produce sufficient quantities of the ribozyme to destroy USF1 messages and inhibit translation. Since ribozymes unlike antisense molecules, are catalytic, a lower intracellular concentration is generally required for efficiency. Ribozyme-mediated RNA repair is another therapeutic option applying ribozyme technologies (Watanabe & Sullenger 2000, Adv. Drug Deliv. Rev. 44: 109-118) and may also be useful for the purpose of the present invention.

The term “aptamer” refers to RNA and also DNA molecules capable of binding target proteins with high affinity and specificity, comparable with the affinity and specificity of monoclonal antibodies. Methods for obtaining or identifying aptamers specific for a desired target are known in the art. Preferably, these methods may be based on the “systematic evolution of ligands by exponential enrichment” (SELEX) process (Ellington and Szostak, Nature, 1990, 346: 818-822; Tuerk and Gold, 1990, Science 249: 505-510; Fitzwater & Polisky, 1996, Methods Enzymol. 267: 275-301). Various chemical modifications, for example the use of 2′-fluoropyrimidines in the starting library and the attachment of a polyethylene glycol to the 5′ end of an aptamer can be used to ensure stability and to enhance bioavailability of aptamers (see e.g. Toulme 2000, Current Opinion in Molecular Therapeutics 2: 318-324).

The inhibitor can also be an antibody or fragment or derivative thereof. As used herein, the term “antibody or fragment or derivative thereof” relates to a polyclonal antibody, monoclonal antibody, chimeric antibody, single chain antibody, single chain Fv antibody, human antibody, humanized antibody or Fab fragment specifically binding to USF1.

Finally, the present invention relates to the use of an activator of expression of USF1 gene for the preparation of a pharmaceutical composition for the treatment of hyperlipidemias and/or dyslipidemias including familial combined hyperlipidemia (FCHL), hypercholesterolemia, hypertriglyceridemia, hypoalphalipoproteinemia, hyperapobetalipoproteinemia (hyperapoB), familial dyslipidemic hypertension (FDH), metabolic syndrome, type 2 diabetes mellitus, coronary heart disease, atherosclerosis or hypertension, wherein said activator is a small molecule

### REFERENCES

1. Goldstein, J. L., Schrott, H. G., Hazard, W. R., Bierman, E. L. & Motulsky, A. G. Hyperlipidemia in coronary heart disease II. Genetic analysis of lipid levels in 176 families and delineation of a new inherited disorder, combined hyperlipidemia. J. Clin. Invest. 52, 1544-1568 (1973).
2. Nikkilä, E. A. & Aro, A. Family study of serum lipids and lipoproteins in coronary heart disease. Lancet 1, 954-959 (1973).
3. Genest, J. J. Jr. et al., Familial lipoprotein disorders in patients with premature coronary artery disease. Circulation 85, 2025-2033 (1992).
4. Pajukanta, P. et al. Linkage of familial combined hyperlipidemia to chromosome 1q21-q23. Nat. Genet. 18, 369-373 (1998).
5. Coon, H. et al. Replication of linkage of familial combined hyperlipidemia to chromosome 1q with additional heterogeneous effect of apolipoprotein A-I/CIII/A-IV locus: the NHLBI family heart study. Arterioscler. Thromb. Vasc. Biol. 20, 2275-2280 (2000),
6. Pei, W. et al. Support for linkage of familial combined hyperlipidemia to chromosome 1q21-q23 in Chinese and German families. Clin. Genet. 57, 29-34 (2000).
7. Allayee, A. et al. Locus for Elevated Apolipoprotein B Levels on Chromosome 1p31 in Families with Familial Combined Hyperlipidemia. Circ. Res. 90, 926-931 (2002).
8. Aouizerat, B. E. et al. A genome scan for familial combined hyperlipidemia reveals evidence of linkage with a locus on chromosome 11. Am. J. Hum. Genet. 65, 397-412 (1999).
9. Pajukanta, P. et al. Genomewide scan for familial combined hyperlipidemia genes in Finnish families, suggesting multiple susceptibility loci influencing triglyceride, cholesterol and apolipoprotein B levels. Am. J. Hum. Genet. 64, 1453-1463 (1999).
10. Elbein, S. C., Hoffman, M. D., Teng, K., Leppert, M. F. & Hasstedt, S. J. A genome-wide search for type 2 diabetes susceptibility genes in Utah Caucasians. Diabetes 48, 1175-1182 (1999).
11. Hanson, R. L. et al. An autosomal genomic scan for loci linked to type II diabetes mellitus and body-mass index in Pima Indians. Am. J. Hum. Genet. 634, 1130-1138 (1998).
12. Vionnet, N., Hani, El.-H., Dupont, S., Gallina, S., Francke, S. & Dotte, S. Genomewide search for type 2 diabetes-susceptibility genes in French whites: evidence for a novel susceptibility locus for early-onset diabetes on chromosome 3q-qter and independent replication of a type 2-diabetes locus on chromosome 1q21-q24. Am. J. Hum. Genet. 67, 1470-1480 (2000).
13. Wiltshire, S. et al. A genomewide scan for loci predisposing to type 2 diabetes in a U.K. population (the Diabetes UK Warren 2 Repository): analysis of 573 pedigrees provides independent replication of a susceptibility locus on chromosome 1q. Am. J. Hum. Genet. 69, 553-569 (2001).
14. Hsuch, W. C. et al. Genome-wide and fine-mapping linkage studies of type 2 diabetes and glucose traits in the Old Order Amish: evidence for a new diabetes locus on chromosome 14q11 and confirmation of a locus on chromosome 1q21-q24. Diabetes 52, 550-507 (2003).
15. Watanabe, R. M. et al. The Finland-United States investigation of non-insulin-dependent diabetes mellitus genetics (FUSION) study. II. An autosomal genome scan for diabetes-related quantitative-trait loci. Am. J. Hum. Genet. 67, 1186-1200 (2000).
16. Castellani, L. W. et al. Mapping a gene for combined hyperlipidaemia in a mutant mouse strain. Nat. Genet. 18, 374-377 (1998).
17. Bodnar, J. S. et al. Positional cloning of the combined hyperlipidemia gene Hyplip1. Nat. Genet. 30, 110-116 (2002).
18. Salero, E., Gimenez, C. & Zafra, F. Identification of a non-canonical E-box motif as a regulatory element in the proximal promoter region of the apolipoprotein E gene. Biochem. J. 370, 979-986 (2003).
19. Portois, L., Tastenoy, M., Viollet, B. & Svoboda, M. Functional analysis of the glucose response element of the rat glucagon receptor gene in insulin-producing INS-1 cells. Biochim. Biophys. Acta. 1574, 175-186 (2002).
20. Yang, X. P. et al. The E-box motif in the proximal ABCA1 promoter mediates transcriptional repression of the ABCA1 gene. J. Lipid. Res. 43, 297-306 (2002).
21. Smith, F. et al., Transcriptional regulation of adipocyte hormone-sensitive lipase by glucose. Diabetes 51, 293-300 (2002).
22. Casado, M., Vallet, V. S., Kahn, A. & Vaulont, S. Essential role in vivo of upstream stimulatory factors for a normal dietary response of the fatty acid synthase gene in the liver. J. Biol. Chem. 274, 2009-2013 (1999).
23. Ribeiro, A., Pastier, D., Kardassis, D., Chambaz, J. & Cardot, P. Cooperative binding of upstream stimulatory factor and hepatic nuclear factor 4 drives the transcription of the human apolipoprotein A-II gene. J. Biol. Chem. 274, 1216-1225 (1999).
24. Iynedjian, P. B. Identification of upstream stimulatory factor as transcriptional activator of the liver promoter of the glucokinase gene. Biochem. J. 333, 705-712 (1998).
25. Soro, A., Jauhiainen, M., Ehnholm, C. & Taskinen, M. R. Determinants of low HDL levels in familial combined hyperlipidemia. J. Lipid Res. 44, 1536-1544 (2003).
26. Risch, N. & Teng, J. The relative power of family-based and case-control designs for linkage disequilibrium studies of complex human diseases 1. DNA pooling. Genome Res. 8, 1273-1288 (1998).
27. Terwilliger, J. D. & Ott, J. A haplotype-based ‘haplotype relative risk’ approach to detecting allelic associations. Hum. Hered. 42, 337-346 (1992).
28. Spielman, R. S., McGinnis, R. E. & Ewens, W. J. Transmission test for linkage disequilibrium: the insulin gene region and insulin-dependent diabetes mellitus (IDDM). Am. J. Hum. Genet. 52, 506-516 81993).
29. Sinsheimer, J. S., Blangero, J. & Lange, K. Gamete competition models. Am. J. Hum. Genet. 66, 1168-1172 (2000).
30. Peltonen, L., Pekkarinen, P. & Aaltonen, J. Messages from an isolate: lessons from the Finnish gene pool. Biol. Chem. 376, 697-704 (1995).
31. Rioux, J. D. et al. Genetic variation in the 5q31 cytokine gene cluster confers susceptibility to Crohn disease. Nat. Genet. 29, 223-228 (2001).
32. Vallet, V. S. et al. Differential roles of upstream stimulatory factors 1 and 2 in the transcriptional response of liver genes to glucose. J. Biol. Chem. 7, 20175-20179 (1998).
33. Wang, D. & Sul, H. S. Upstream stimulatory factor binding to the E-box at −65 is required for insulin regulation of the fatty acid synthase promoter. J. Biol. Chem. 272, 26367-26374 (1997).
34. Pan, L. et al. Critical roles of a cyclic AMP responsive element and an E-box in regulation of mouse renin gene expression. J. Biol. Chem. 276, 45530-45538 (2001).
35. Yanai, K., Saito, T., Hirota, K., Kobayashi, H., Murakami, K. & Fukamizu, A.

Molecular variation of the human angiotensinogen core promoter element located between the TATA box and transcription initiation site affects its transcriptional activity. J. Biol. Chem. 272, 30558-30562 (1997).
36. Barton, E. S. et al. Junction adhesion molecule is a receptor for reovirus. Cell 104, 441-451 (2001).
37. Ostermann, G., Weber, K. S., Zernecke, A., Schroder, A. & Weber, C. JAM-1 is a ligand of the beta(2) integrin LFA-1 involved in transendothelial migration of leukocytes. Nat. Immunol. 3, 151-158 (2002).
38. Enattah, N. S., Sahi, T., Savilahti, E., Terwilliger, J. D., Peltonen, L. & Jarvela, I. Identification of a variant associated with adult-type hypolactasia. Nat. Genet. 30, 233-237 (2002).
39. Vakkilainen, J., Jauhiainen, M., Ylitalo, K., Nuotio, I. O., Viikari, J. S., Ehnholm, C. & Taskinen, M. R. LDL particle size in familial combined hyperlipidemia: effects of serum lipids, lipoprotein-modifying enzymes, and lipid transfer proteins. J. Lipid Res. 43, 598-603 (2002).
40. Pielberg, G., Olsson, C., Syvanen, A.-C. & Andersson, L. Unexpectedly High Allelic Diversity at the KIT Locus Causing Dominant White Color in the Domestic Pig. Genetics 160, 305-311 (2002).
41. The Gene Ontology Consortium Gene Ontology: tool for the unification of biology. Nat. Genet. 25, 25-29 (2000).
42. Hosack, D A., Dennis, G. Jr., Sherman, B. T., Lane, H. C., Lempicki, R. A. Identifying biological themes within lists of genes with EASE. Genome Biol. 4, R70.1-R70.8 (2003).
43. Lathrop, G. M., Lalouel, J.-M., Julier, C. A. & Ott, J. Strategies for multilocus linkage analysis in humans. Proc. Natl. Acad. Sci. USA 81, 3443-3446 (1984).
44. Cottingham, R. W., Jr, Idury, R. M. & Schäffer, A. A. Faster sequential genetic linkage computations. Am. J. Hum. Genet. 53, 252-263 (1993).
45. Schäffer, A. A., Gupta, S. K., Shriram, K. & Cottingham, R. W., Jr. Avoiding recomputation in linkage analysis. Hum. Hered. 44, 225-237 (1994).
46. Goring, H. H. & Terwilliger, J. D. Gene mapping in the 20th and 21st centuries: statistical methods, data analysis, and experimental design. Hum. Biol. 72, 63-132 (2000a).
47. Göring, H. H. & Terwilliger, J. D. Linkage analysis in the presence of errors III: marker loci and their map as nuisance parameters. Am. J. Hum. Genet. 66, 1298-1309 (2000b).
48. Terwilliger, J. D. A powerful likelihood method for the analysis of linkage disequilibrium between trait loci and one or more polymorphic marker loci. Am. J. Hum. Genet. 56, 777-787 (1995).
49. Laird, N., Horvath, S. & Xu, X. Implementing a unified approach to family based tests of association. Genet. Epidemiol. 19, 36-42 (2000).
50. Martin, E. R., Bass, M. P., Gilbert, J. R., Pericak-Vance, M. A., & Hauser E R. Genet. Epidemiol. (2003, in press)

### ADDITIONAL REFERENCES 1A TO 33A

1. Goldstein, J. L., Schrott, H. G., Hazzard, W. R., Bierman, E. L. & Motulsky, A. G. Hyperlipidemia in coronary heart disease. II. Genetic analysis of lipid levels in 176 families and delineation of a new inherited disorder, combined hyperlipidemia. J Clin Invest 52, 1544-68 (1973).
2. Nikkila, E. A. & Aro, A. Family study of serum lipids and lipoproteins in coronary heart-disease. Lancet 1, 954-9 (1973).
3. Wojciechowski, A. P. et al. Familial combined hyperlipidaemia linked to the apolipoprotein AI-CII-AIV gene cluster on chromosome 11q23-q24. Nature 349, 161-4 (1991).
4. Aouizerat, B. E. et al. Linkage of a candidate gene locus to familial combined hyperlipidemia: lecithin:cholesterol acyltransferase on 16q. Arterioscler Thromb Vasc Biol 19, 2730-6 (1999).
5. Pajukanta, P. et al. Genomewide scan for familial combined hyperlipidemia genes in finnish families, suggesting multiple susceptibility loci influencing triglyceride, cholesterol, and apolipoprotein B levels. Am J Hum Genet 64, 1453-63 (1999).
6. Pajukanta, P. et al. Familial combined hyperlipidemia is associated with upstream transcription factor 1 (USF1). Nat Genet 36, 371-6 (2004).
7. Putt, W. et al. Variation in USF1 shows haplotype effects, gene: gene and gene: environment associations with glucose and lipid parameters in the European Atherosclerosis Research Study II. Hum Mol Genet 13, 1587-97 (2004).
8. Casado, M., Vallet, V. S., Kahn, A. & Vaulont, S. Essential role in vivo of upstream stimulatory factors for a normal dietary response of the fatty acid synthase gene in the liver. J Biol Chem 274, 2009-13 (1999).
9. Ribeiro, A., Pastier, D., Kardassis, D., Chambaz, J. & Cardot, P. Cooperative binding of upstream stimulatory factor and hepatic nuclear factor 4 drives the transcription of the human apolipoprotein A-II gene. J Biol Chem 274, 1216-25 (1999).
10. Groenen, P. M. et al. Structure, sequence, and chromosome 19 localization of human USF2 and its rearrangement in a patient with multicystic renal dysplasia. Genomics 38, 141-8 (1996).
11. Horikawa, Y. et al. Genetic variation in the gene encoding calpain-10 is associated with type 2 diabetes mellitus. Nat Genet 26, 163-75 (2000).
12. Rioux, J. D. et al. Genetic variation in the 5q31 cytokine gene cluster confers susceptibility to Crohn disease. Nat Genet 29, 223-8 (2001).
13. Yang, X. P. et al. The E-box motif in the proximal ABCA1 promoter mediates transcriptional repression of the ABCA1 gene. J Lipid Res 43, 297-306 (2002).
14. Yanai, K. et al. Molecular variation of the human angiotensinogen core promoter element located between the TATA box and transcription initiation site affects its transcriptional activity. J Biol Chem 272, 30558-62 (1997).
15. Salero, E., Gimenez, C. & Zafra, F. Identification of a non-canonical E-box motif as a regulatory element in the proximal promoter region of the apolipoprotein E gene. Biochem J 370, 979-86 (2003).
16. Nowak, M. et al. Insulin mediated down-regulation of the Apolipoprotein A5 gene expression through the Phosphatidylinositol 3-kinase pathway: Role of the Upstream Stimulatory Factor. Molecular and Cellular Biology (2004, accepted).
17. Wallace, T. M., Levy, J. C. & Matthews, D. R. Use and abuse of HOMA modeling. Diabetes Care 27, 1487-95 (2004).
18. Lopez-Casillas, F., Ponce-Castaneda, M. V. & Kim, K. H. In vivo regulation of the activity of the two promoters of the rat acetyl coenzyme-A carboxylase gene. Endocrinology 129, 1049-58 (1991).
19. Mootha, V. K. et al. PGC-1 alpha-responsive genes involved in oxidative phosphorylation are coordinately downregulated in human diabetes. Nat Genet 34, 267-73 (2003).
20. Dyrskjot, L. et al. Identifying distinct classes of bladder carcinoma using microarrays. Nat Genet 33, 90-6 (2003).
21. Zhao, Q. et al. Essential Role of vascular endothelial growth factor in angiotensin II-induced vascular inflammation and remodeling. Hypertension 44, 264-70 (2004).
22. Oram, J. F. ATP-binding cassette transporter A1 and cholesterol trafficking. Curr Opin Lipidol 13, 373-81 (2002).
23. Brooks-Wilson, A. et al. Mutations in ABC1 in Tangier disease and familial high-density lipoprotein deficiency. Nat Genet 22, 336-45 (1999).
24. Zhang, S. H., Reddick, R. L., Piedrahita, J. A. & Maeda, N. Spontaneous hypercholesterolemia and arterial lesions in mice lacking apolipoprotein E. Science 258, 468-71 (1992).
25. Beisiegel, U., Weber, W. & Bengtsson-Olivecrona, G. Lipoprotein lipase enhances the binding of chylomicrons to low density lipoprotein receptor-related protein. Proc Natl Acad Sci USA 88, 8342-6 (1991).
26. Mahley, R. W. Apolipoprotein E: cholesterol transport protein with expanding role in cell biology. Science 240, 622-30 (1988).
27. Shimano, H. et al. Overexpression of apolipoprotein E in transgenic mice: marked reduction in plasma lipoproteins except high density lipoprotein and resistance against diet-induced hypercholesterolemia. Proc Natl Acad Sci USA 89, 1750-4 (1992).
28. Wilhelm, M. G. & Cooper, A. D. Induction of atherosclerosis by human chylomicron remnants: a hypothesis. J Atheroscler Thromb 10, 132-9 (2003).
29. Kypreos, K. E., Li, X., van Dijk, K. W., Havekes, L. M. & Zannis, V. I. Molecular mechanisms of type III hyperlipoproteinemia: The contribution of the carboxyterminal domain of ApoE can account for the dyslipidemia that is associated with the E2/E2 phenotype. Biochemistry 42, 9841-53 (2003).
30. Rail, S. C., Jr. & Mahley, R. W. The role of apolipoprotein E genetic variants in lipoprotein disorders. J Intern Med 231, 653-9 (1992).
31. Heeren, J. et al. Impaired recycling of apolipoprotein E4 is associated with intracellular cholesterol accumulation. J Biol Chem (2004).
32. Ha, J. et al. Cloning of human acetyl-CoA carboxylase cDNA. Eur J Biochem 219, 297-306 (1994).
33. Soro, A. et al. Genome scans provide evidence for low-HDL-C loci on chromosomes 8q23, 16q24.1-24.2, and 20q13.11 in Finnish families. Am J Hum Genet 70, 1333-40 (2002).

The figures show:

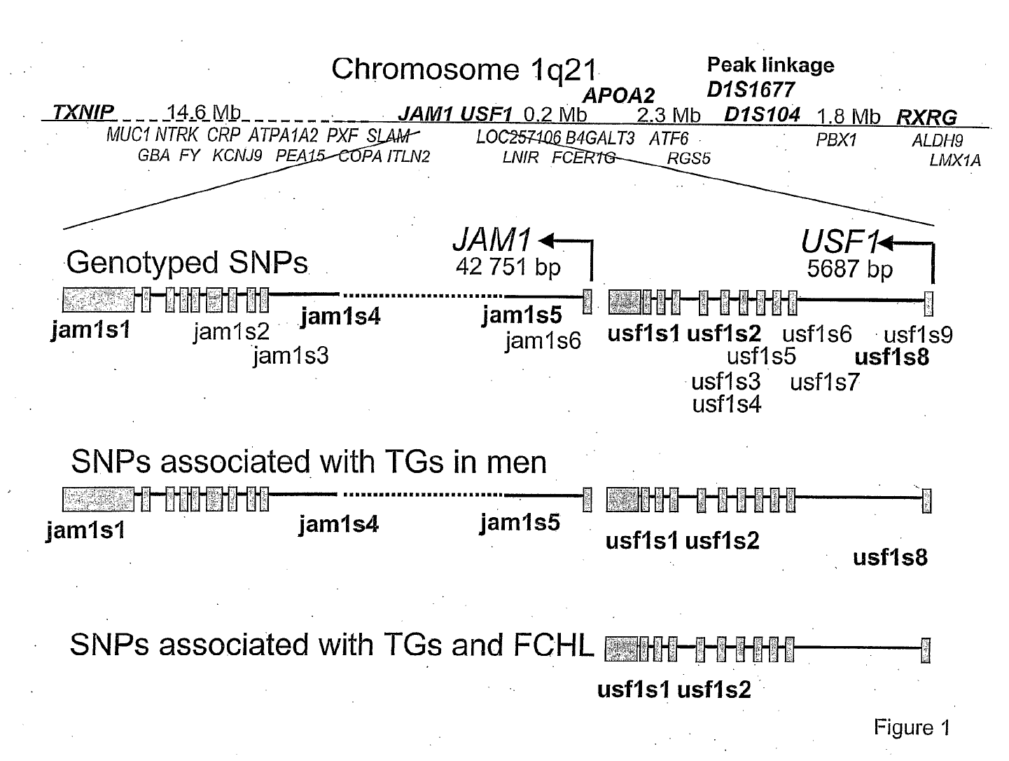

FIG. 1: Schematic overview of the associated region on 1q21. Genes for which we genotyped SNPs as well as the locations of the peak linkage markers D1S104 and D1S1677 (Pajukanta et al. 1998) are shown in the uppermost part. The genes indicated in bold were also sequenced. Next part shows the SNPs genotyped for JAM1 and USF1 (see Table 2 for distances, rs numbers and LD clusters of these SNPs). The second to lowest part indicates the SNPs associated with TGs in men, and the lowest part the SNPs associated with FCHL and TGs in all family members.

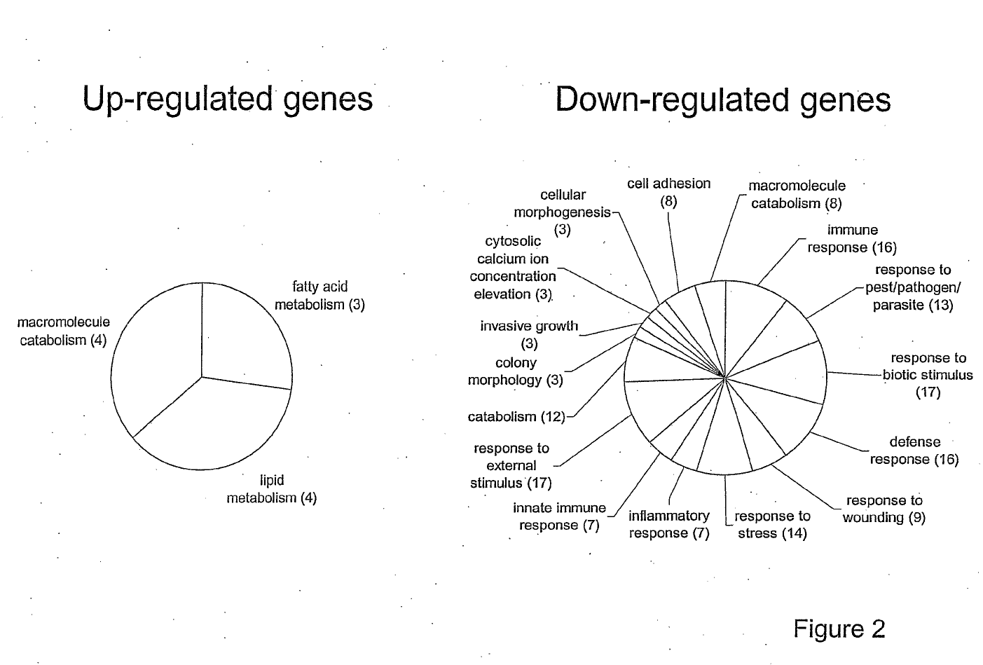

FIG. 2: Distribution of genes according to functional category for the 16 up-regulated and 60 down-regulated genes for which annotation information for the gene ontology (GO) class Biological process was available. Only categories scoring a statistically significant EASE-score (<0.05) for over-representation are shown. Complete results of the EASE analysis including the corresponding EASE scores (p-values) and the lists of genes in every significant category are given in the Supplementary Table 3a-b.

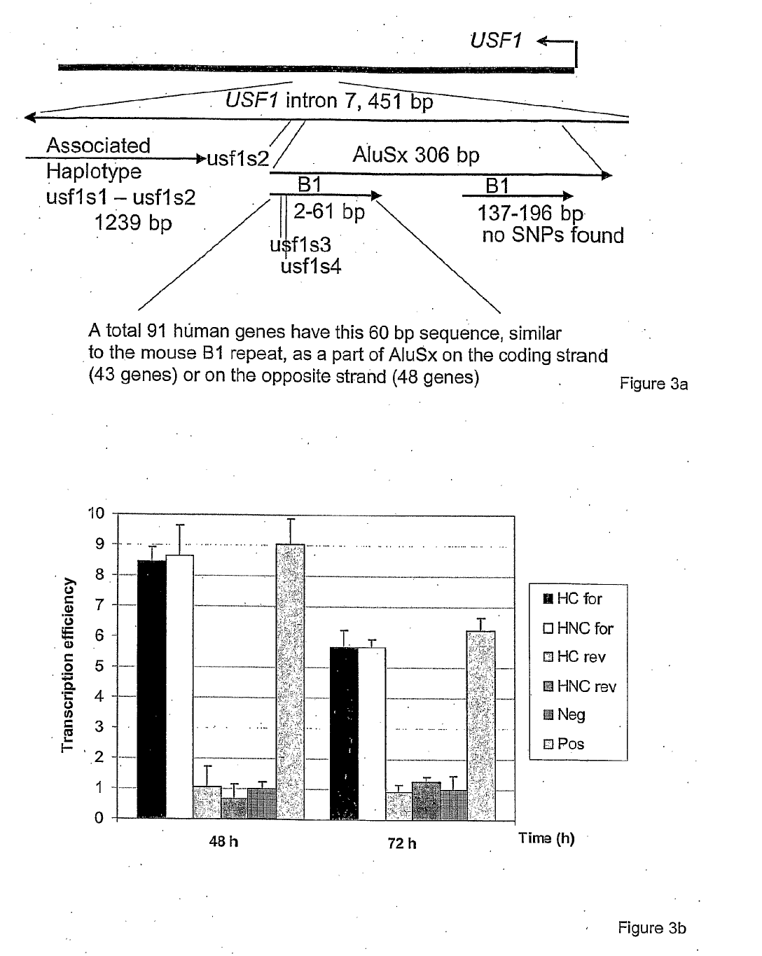

FIG. 3a: Intron 7 of USF1 harbors the 60-bp sequence shared by the 91 USF1-similarity genes. Parts (2-61 bp and 137-196 bp) of the AluSx repeat in intron 7 of USF1 have sequence similarities with the mouse B1 repeat. A total of 91 human genes, including USF1, have this 60-bp part of AluSx located either on the coding strand (43 genes) or on the opposite strand (48 genes). These 91 genes are listed in the Supplementary Table 4.

FIG. 3b: Transcription efficiency of a 268-bp region in intron 7 of USF1 containing the critical 60-bp sequence and the usf1s2 SNP (see FIG. 3a). DNAs from one homozygous susceptibility carrier (haplotype 1-1) and one homozygous non-carrier (2-2) were cloned to the SEAP reporter system in both forward and reverse orientations. HC for and HC rev indicate constructs of a haplotype carrier (1-1) DNA in forward and reverse orientations; HNC for and HNC rev indicate constructs of a haplotype non-carrier (2-2) DNA in forward and reverse orientations. Culture media from cells transfected with the pSEAP2-Basic vector was used as a negative control (Neg) and culture media from cells transfected with the pSEAP2-Control vector as a positive control (Pos), respectively. The monitoring of the SEAP protein was performed 48 and 72 hours post-transfection. Error bars represent SD of one experiment done in triplicate. The size of the bar indicates the increase in transcriptional activity when compared to the negative control which is set to 1.

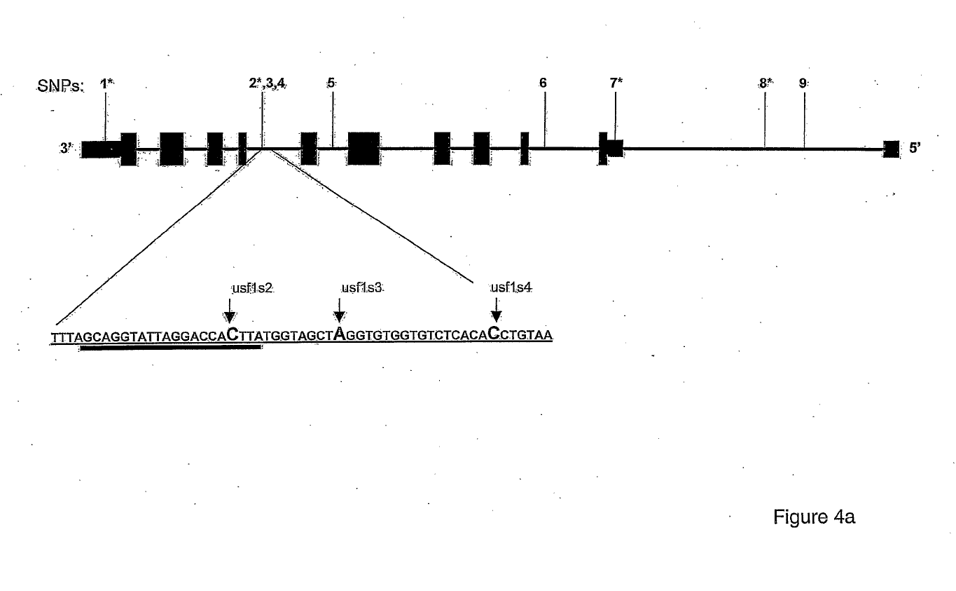

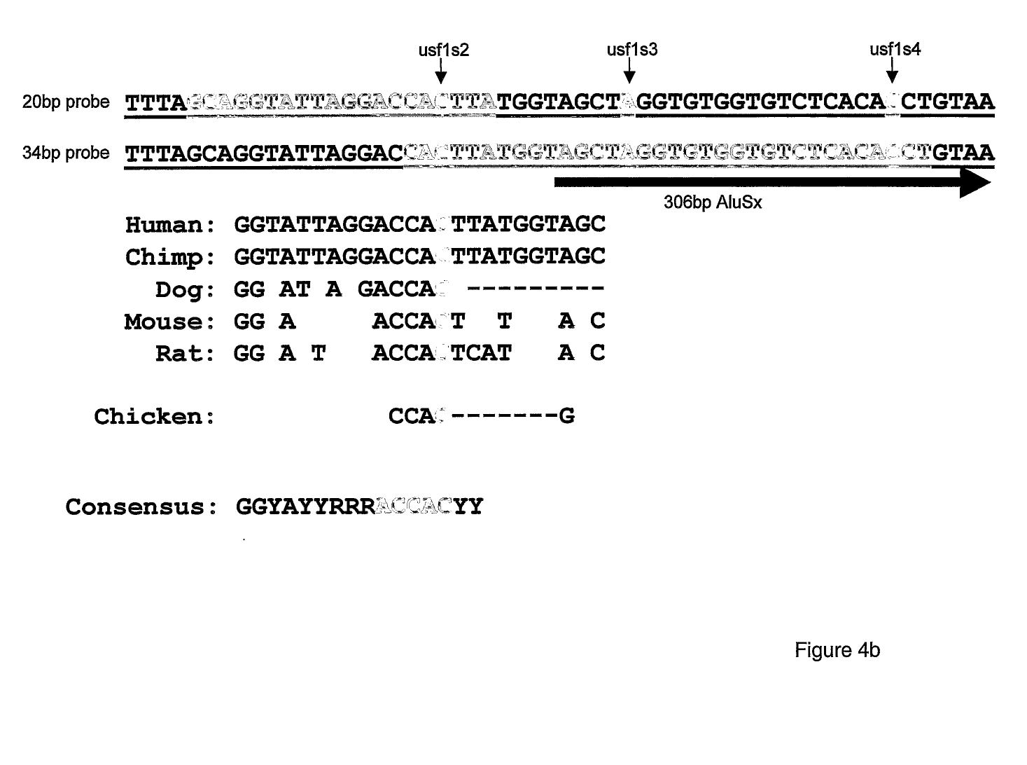

FIG. 4a: Schematic view of the 6.7 kb USF1 gene. Exons are depicted as thick boxes, UTRs as thinner boxes and introns as lines. Genotyped USF1 SNPs are marked above the gene with associating SNPs indicated with asterixes. A segment of intron 7 is amplified to show the location of the sequence (black bar), used to generate the 20-mer probe used in the EMSA. Nearby SNPs are indicated with larger font and arrows.

FIG. 4b: Cross-species conservation and EMSA probes. Two probes were constructed that both were capable of producing a shift in the EMSA; One of length 34 bp and the other 20 bp. The 34-mer probe contained all three SNPs from this intron 7 region, whereas the 20-mer probe only contained the critical usf1s2 SNP. Below is shown the cross-species sequence conservation and the consensus sequence. Y stands for pyrimidine and R for purine. Notably the nucleotide at usf1s2 itself is fully conserved, the risk allele representing the ancestral allele.

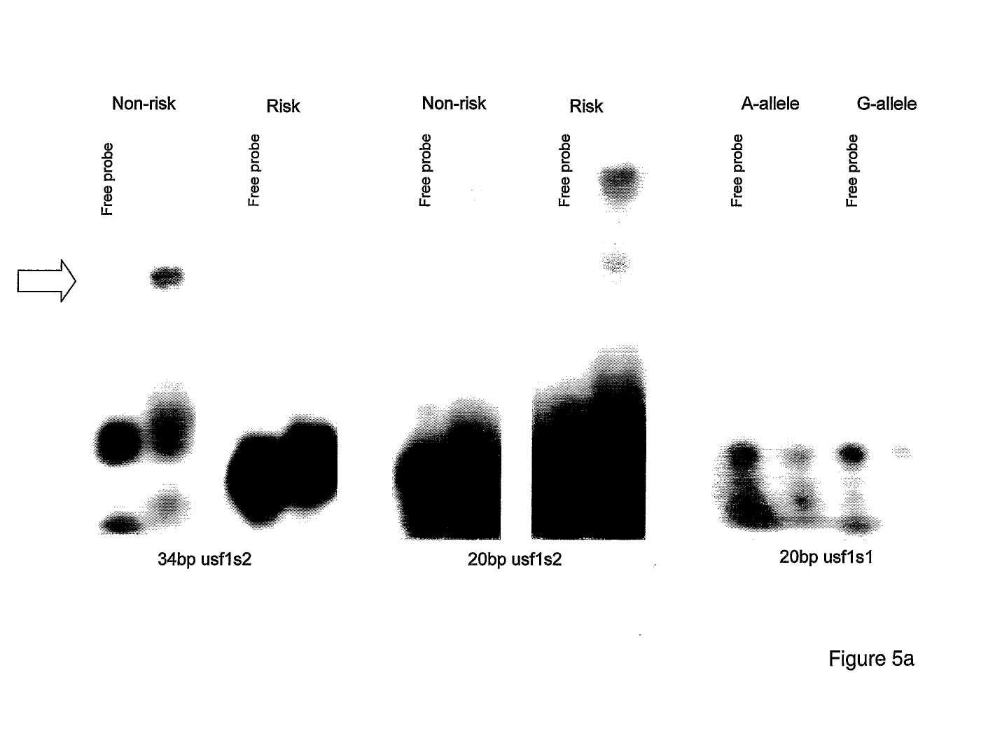

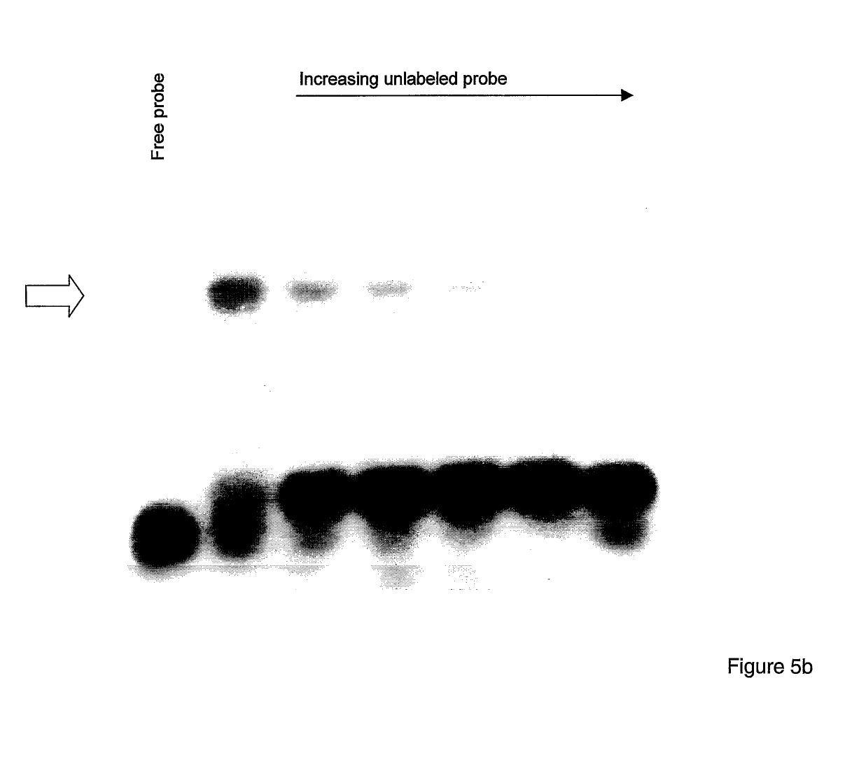

FIG. 5a: EMSA results show that both the 34 bp and the 20 bp probe around usf1s2 bind nuclear protein(s) from HeLa cell extract. The different usf1s2 allelic variants of both probe sets produce a gel-shift, marked by an arrow. Conversely, neither variant of the 20 bp probe representing the sequence around usf1s1 in the 3′UTR is capable of producing a gel-shift.

FIG. 5b: The specificity of the binding of nuclear protein(s). The 34 bp probe representing the sequence around usf1s2 produces a strong gel-shift which can be gradually competed with the addition of increasing molar concentrations of unlabeled probe.

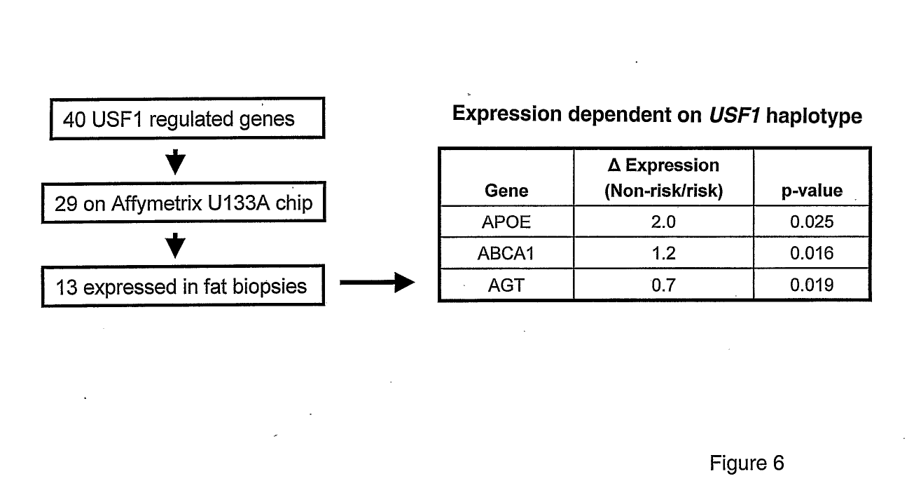

FIG. 6: Schematic overview of the identification of the significantly differentially regulated USF1-controlled genes. The initial list of 40 genes was narrowed down to the 13 that were expressed in the fat biopsies. Of these, three important metabolic genes were differentially expressed at steady state between individuals carrying the risk or non-risk haplotype of USF1. P-values are from a two-sample t-test with no assumption of equal variance.

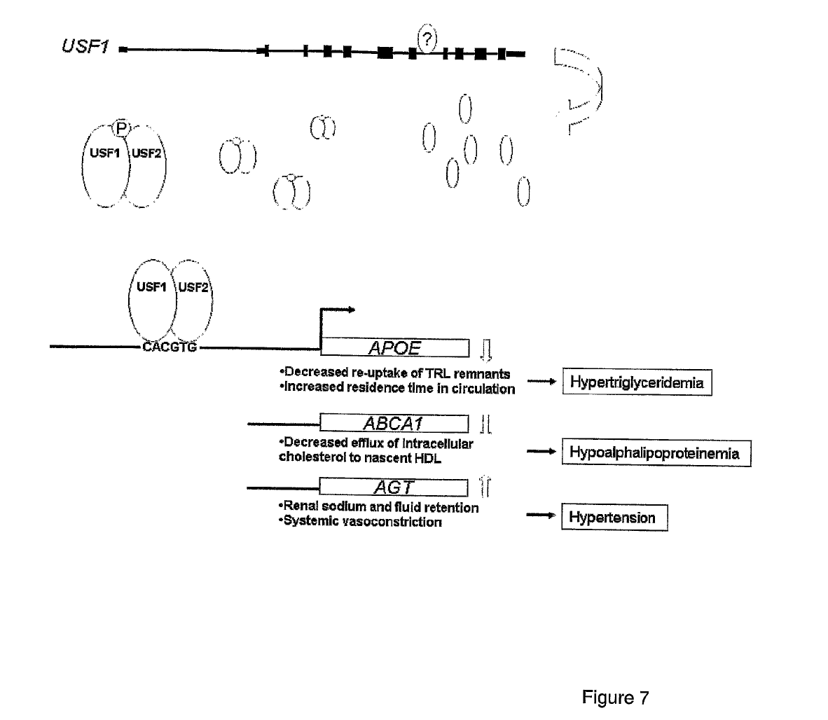

FIG. 7: Schematic representation of the mechanism of allele-specific regulation of the USF1 transcript levels and probable consequences of the variations in the amount of USF1 protein. Protein(s) bind a regulatory sequence in intron 7 of USF1 and affect the level of transcription. USF1 dimerizes (most often with USF2) and binds an E-box sequence in the promoter of numerous genes to activate their transcription in response to signals such as glucose and dietary carbohydrates. Post-translational control of USF1 activity is mediated by phosphorylation of the dimer which precludes its binding to the E-box motif¹⁶. The observed decrease in the transcript level of downstream genes, if reflected at the polypeptide level, would result in changes highly relevant for dyslipidemias and the metabolic syndrome.

The examples illustrate the invention.

### EXAMPLE 1

### Experimental Outline of Examples 2 to 5

All analyzed FCHL families had a proband with severe CHD and lipid phenotype, and on average 5-6 FCHL affected family members. These FCHL families exhibiting extreme and well-defined disease phenotypes were analyzed to identify the underlying gene contributing to FCHL on 1q21. We selected a regional candidate gene approach and sequenced four functionally relevant regional candidate genes on 1q21. The TXNIP, USF1, retinoid X receptor gamma (RGRG), and apolipoprotein A2 (APOA2) genes were sequenced to identify all possible variants. Of these, TXNIP initially represented the most promising positional candidate gene, because it has been shown to underlie the combined hyperlipidemia phenotype in mice¹⁷. The three additional regional genes were selected for sequencing based on their functional candidacy and close location (<2.5 Mb) to the original peak linkage markers, D1S104 and D1S1677 (FIG. 1). In parallel, we employed a functionally unbiased, genetic approach, where an initial set of SNPs for genes around the peak linkage markers were tested for association. A total of 60 SNPs were genotyped for 26 genes on 1q21. Fifty of these SNPs were located within 5.8 Mb, flanking D1S104 and D1S1677. All 60 SNPs were genotyped in 238 family members of 42 FCHL families, including the 31 families of the original linkage study⁴, and 10 most promising SNPs in the extended sample of 721 family members from 60 FCHL families (see below). The results of the 60 SNPs are shown in the Supplementary Table 1.

SUPPLEMENTARY TABLE 1
RESULTS OF THE 60 GENOTYPED SNPs OBTAINED IN TWO-POINT LINKAGE AND ASSOCIATION
ANALYSES. A TOTAL OF FOUR TRAITS WERE ANALYZED: ALL INDIVIDUALS FOR THE FCHL AND
TG TRAITS, AS WELL AS AFFECTED MALES FOR THE FCHL AND TG TRAITS.

| | | | FCHL | | FCHL men | | TG | | TG men | |
|---|---|---|---|---|---|---|---|---|---|---|
| | | Distance | Linkage | HHRR | Linkage | HHRR | Linkage | HHRR | Linkage | HHRR |
| Gene | SNP | in · bp | Lod | P-value | Lod | P-value | Lod | P-value | Lod | P-value |
| TXNIP | rs2236567 | 425 | 0.4 | ns | 0.0 | ns | 0.2 | ns | 0.3 | ns |
| TXNIP | Nf | 1272 | 0.3 | ns | 0.0 | ns | 0.2 | ns | 0.0 | ns |
| TXNIP | rs9245 | 3039 | 0.3 | ns | 0.1 | ns | 0.4 | ns | 0.8 | ns |
| TXNIP | rs7211 | 8869064 | 0.6 | ns | 0.1 | ns | 0.3 | ns | 0.0 | ns |
| MUC1 | rs1611774 | 4214 | 0.2 | ns | 0.1 | ns | 1.1 | ns | 1.4 | ns |
| MUC1 | rs4072037 | 22637 | 0.0 | ns | 0.0 | ns | 0.0 | ns | 0.1 | ns |
| GBA | rs1800473 | 1661529 | 0.4 | ns | 0.0 | ns | 0.5 | ns | 0.2 | ns |
| NTRK1 | rs6334 | 2762 | 0.2 | ns | 0.0 | ns | 0.2 | ns | 0.1 | ns |
| NTRK1 | rs6337 | 2326359 | 0.0 | ns | 0.0 | ns | 0.2 | ns | 0.1 | ns |
| FY | rs12075 | 507737 | 1.0 | ns | 1.0 | ns | 1.5 | ns | 0.8 | ns |
| CRP | rs1130864 | 367299 | 0.0 | ns | 0.0 | ns | 0.2 | ns | 0.3 | ns |
| KCNJ9 | rs4656876 | 521 | 0.0 | ns | 0.0 | ns | 0.4 | ns | 0.1 | ns |
| KCNJ9 | rs2180752 | 7051 | 0.0 | ns | 0.0 | ns | 0.0 | ns | 0.0 | ns |
| KCNJ9 | rs2737705 | 288 | 0.0 | ns | 0.0 | ns | 0.4 | ns | 0.6 | ns |
| KCNJ9 | rs2753268 | 302 | 0.0 | ns | 0.0 | ns | 0.2 | ns | 1.0 | ns |
| KCNJ9 | Nf | 38761 | 0.1 | ns | 0.0 | ns | 0.2 | ns | 0.5 | ns |
| ATP1A2 | rs2295623 | 12474 | 0.0 | ns | 0.0 | ns | 0.0 | ns | 0.0 | ns |
| ATP1A2 | Nf | 71117 | 0.0 | ns | 0.0 | ns | 0.0 | ns | 0.0 | ns |
| PEA15 | rs680083 | 66279 | 0.0 | ns | 0.0 | ns | 0.0 | ns | 0.0 | ns |
| PXF | rs10594 | 56231 | 0.4 | ns | 0.1 | ns | 0.2 | ns | 0.4 | ns |
| COPA | rs1802778 | 276599 | 0.0 | ns | 0.0 | ns | 0.0 | ns | 0.0 | ns |
| SLAMF1 | rs1061217 | 337887 | 0.5 | ns | 0.0 | ns | 0.2 | ns | 0.3 | ns |
| ITLN2 | rs1556519 | 24927 | 0.9 | ns | 0.1 | ns | 1.0 | ns | 1.1 | ns |
| Flanking | rs2246485 | 25395 | 1.1 | ns | 0.0 | ns | 1.1 | ns | 0.3 | ns |
| F11R | | | | | | | | | | |
| F11R/f11rs1 | rs836 | 1361 | 1.7 | ns | 0.1 | ns | 2.8 | ns | 0.9 | 0.03 |
| F11R/f11rs2 | rs790056 | 1561 | 0.9 | ns | 0.0 | ns | 0.5 | ns | 1.1 | ns |
| F11R/f11rs3 | rs790055 | 25608 | 0.7 | ns | 0.0 | ns | 0.4 | ns | 0.3 | ns |
| F11R/f11rs4 | hCV1459766 | 10572 | 1.8 | ns | 0.1 | ns | 2.7 | ns | 0.4 | ns |
| F11R/f11rs5 | rs4339888 | 1246 | 2.2 | ns | 0.1 | ns | 3.6 | ns | 0.6 | 0.02 |
| F11R/f11rs6 | rs3766383 | 951 | 0.0 | ns | 0.0 | ns | 0.0 | ns | 0.0 | ns |
| USF1/usf1s1 | rs3737787 | 1239 | 3.3 | ns | 0.3 | 0.04 | 2.1 | ns | 2.0 | 0.0009 |
| USF1/usf1s2 | rs2073658 | 12 | 2.0 | ns | 0.0 | 0.04 | 1.5 | ns | 1.8 | 0.002 |
| USF1/usf1s3 | rs2516841 | 17 | 1.3 | ns | 0.0 | ns | 1.8 | ns | 0.4 | ns |
| USF1/usf1s4 | rs2073657 | 526 | 0.4 | ns | 0.1 | ns | 1.1 | ns | 0.4 | ns |
| USF1/usf1s5 | rs2516840 | 1443 | 0.7 | ns | 0.0 | ns | 0.8 | ns | 0.2 | ns |
| USF1/usf1s6 | rs2073653 | 361 | 0.0 | ns | 0.0 | ns | 0.0 | ns | 0.0 | ns |
| USF1/usf1s7 | rs2516839 | 1249 | 0.7 | ns | 0.0 | ns | 2.1 | ns | 1.2 | ns |
| USF1/usf1s8 | rs2516838 | 279 | 0.1 | ns | 0.0 | ns | 0.4 | ns | 0.1 | 0.01 |
| USF1/usf1s9 | rs1556259 | 4391 | 0.0 | ns | 0.0 | ns | 0.0 | ns | 0.0 | ns |
| LOC257106 | rs3813609 | 5724 | 0.1 | ns | 0.0 | ns | 0.8 | ns | 0.1 | ns |
| LOC257106 | Nf | 26087 | 0.1 | ns | 0.1 | ns | 0.1 | ns | 0.3 | ns |
| LNIR | rs1467742 | 283 | 0.0 | ns | 0.0 | ns | 0.0 | ns | 0.0 | ns |
| LNIR | rs1556257 | 2639 | 0.1 | ns | 0.1 | ns | 0.0 | ns | 0.0 | ns |
| LNIR | rs4529727 | 87659 | 0.0 | ns | 0.0 | ns | 0.6 | ns | 0.2 | ns |
| B4GALT3 | rs6779 | 47461 | 0.1 | ns | 0.0 | ns | 0.3 | ns | 0.4 | ns |
| FCER1G | rs3557 | 43 | 0.1 | ns | 0.0 | ns | 0.0 | ns | 0.0 | ns |
| FCER1G | rs11421 | 2593 | 0.1 | ns | 0.0 | ns | 0.3 | ns | 0.0 | ns |
| APOA2 | Nf | 34 | 0.3 | ns | 0.0 | ns | 0.6 | ns | 0.0 | ns |
| APOA2 | Nf | 948 | 1.1 | ns | 0.0 | ns | 1.5 | ns | 0.0 | ns |
| APOA2 | rs5085 | 1172 | 0.1 | ns | 0 0 | ns | 0.0 | ns | 0.0 | ns |
| APOA2 | rs5082 | 645533 | 0.3 | ns | 0.1 | ns | 3.1 | ns | 2.1 | ns |
| ATF6 | CV67448 | 1196247 | 0.0 | ns | 0.0 | ns | 0.0 | ns | 0.0 | ns |
| RGS5 | rs15049 | 1412242 | 0.0 | ns | 0.1 | ns | 0.0 | ns | 0.0 | ns |
| PBX1 | rs2275558 | 122535 | 0.0 | ns | 0.0 | ns | 0.0 | ns | 0.0 | ns |
| PBX1 | rs1057756 | 164453 | 0.0 | ns | 0.2 | ns | 0.2 | ns | 0.1 | ns |
| PBX1 | rs14832 | 561444 | 0.1 | ns | 0.0 | ns | 1.3 | ns | 0.1 | ns |
| RXRG | rs2134095 | 11733 | 0.3 | ns | 0.0 | ns | 1.2 | ns | 0.2 | ns |
| RXRG | rs157870 | 242385 | 0.0 | ns | 0.0 | ns | 0.0 | ns | 0.0 | ns |
| ALDH9A1 | rs12670 | 307375 | 0.8 | ns | 0.0 | ns | 0.9 | ns | 0.0 | ns |
| LMX1A | hCV3194556 | | 0.9 | ns | 0.0 | ns | 0.9 | ns | 0.1 | ns |

Lod scores were obtained in two-point linkage analysis (see methods for details) and p-values in the association analysis using the HHRR test. Nf indicates not found in dbSNP or Celara databases. The SNP information for these SNPs will be submitted to the public database (dbSNP). SNPs indicated in bold were genotyped in the 60 extended FCHL families. All other results were obtained in the 42 nuclear FCHL families. P-values less than 0.05 are also shown in bold,whereas ns indicates non-significant (p-value greater than 0.05).

### EXAMPLE 2

### USF1 Gene as a Candidate Gene

We identified a total of 23 SNPs for the 5687 bp sequence of the USF1 gene (Supplementary Table 2): Three of these were silent variants in exons, and the rest were located in the non-coding regions and in the putative promoter. Eight of the 23 SNPs were novel. Initially, we genotyped three SNPs for the USF1 gene: usf1s1 (exon 11), usf1s2 (intron 7), and usf1 s7 (exon 2) (the corresponding rs numbers for the genotyped SNPs are given in Tables 2-3),

TABLE 1
MULTIPOINT HHRR AND GAMETE COMPETITION
ANALYSES FOR THE SNPs USF1S1
(=Rs3737787) AND USF1s2 (=Rs2073658).

| | FCHL all | TG all | FCHL men | TG men |
|---|---|---|---|---|
| Multi-HHRR | ns (ns) | 0.05 (ns) | 0.009 (ns) | 0.00003 |
| | | | | (0.003) |
| Gamete | 0.00002 | 0.00006 | 0.0004 | 0.0000009 |
| competition | (0.005) | (0.008) | (0.04) | (0.004) |
| asymptotic | | | | |
| p-value | | | | |
| Gamete | 0.00004 | 0.00006 | 0.0004 | 0.00001 |
| competition | | | | |
| (Gene dropping) | | | | |
| empirical p-value | | | | |

All values represent p-values for simultaneous analysis of both SNPs. Ns indicates non-significant. The first presented p-values were obtained in 60 extended FCHL families and the values given in parentheses in 42 nuclear FCHL families. Gene dropping was performed only in the 60 extended FCHL families using at least 50,000 simulations. The segregating haplotype was 1-1 (1 indicates the common allele) in all gamete competition analyses above.

SUPPLEMENTARY TABLE 2
ASSOCIATION AND LINKAGE ANALYSES OF TXNIP WITH FCHL.

| | Analysis of single SNPs | | | | Analysis of |
|---|---|---|---|---|---|
| | | SNP2 | | | combined |
| | SNP1 | −1273 bp | SNP3 | SNP4 | SNPs |
| Method | rs2236567 | C->T | rs9245 | rs7211 | SNP1-2-3-4 |
| Linkage | | | | | |
| LOD | 0.4 (0.14) | 0.3 (0.12) | 0.3 | 0.6 | 1.9 (0.11) |
| | | | (0.20) | (0.10) | |
| ASP | 0.3 | 0.3 | 0.6 | 0.2 | |
| Family-based | | | | | |
| Association | | | | | |
| GAMETE | ns | ns | ns | ns | ns |
| HHRR | ns | ns | ns | ns | ns |
| HBAT | | | | | ns |
| Heterozygosity | 0.11 | 0.10 | 0.11 | 0.12 | |

LOD indicates the maximum lod score of the parametric two-point or multipoint linkage analysis using the MLINK program and a dominant mode of inheritance (recombination fraction is given in parentheses); ASP indicates the lod score obtained in the affected sib-pair analysis; GAMETE indicates the p-values obtained in the Gamete competitionanalysis; HHRR and multi-HHRR the p-values obtained in the haplotype-based haplotype relative risk analysis; and HBAT the p-value for the test between the TXNIP haplotypes and the FCHL trait. Ns indicates non-significant. For the TG trait, the corresponding p-values for all association analyses remained non-significant, and both two-andmultipoint lod scores were <1.5. The numbering of the new SNP2 is based on the genomic sequence of the TXNIP region at the UCSC Genome Browser, July 2003. All of these SNPs were genotyped in the extended sample of 721 family members from 60 FCHL families.

The usf1s1 and usf1s2 provided evidence for linkage in the 42 FCHL families with maximum Iod scores of 3.5 and 2.0 for FCHL, and 3.7 and 2.0 for TGs. Combined analysis of these SNPs also provided some evidence for association with the gamete competition test for both FCHL (p=0.005) and TGs (p=0.008) (Table 1), although the results of individual SNPs were non-significant. We also observed a difference in the allele frequencies between unaffected and affected men, especially with the TG trait. The frequency of minor allele of usf1s1 was 22.0% in TG-affected males and 40% in the unaffected male family members. Since these affected and unaffected family members represent non-independent groups of males, we tested usf1s1 and usf1s2 in TG-affected men using the family-based association method, HHRR, and the gamete competition test: p-values of 0.01 and 0.02 were obtained in the HHRR analysis and 0.008 and 0.02 in the gamete competition test of the 42 nuclear FCHL families (Table 2). The combined analysis of these SNPs yielded a p-value of 0.003 in the HHRR test and 0.004 in the gamete competition test for TGs in men (Table 1).

TABLE 2
ASSOCIATION ANALYSES OF INDIVIDUAL SNPS FOR THE JAM1-USF1
REGION FOR TGS AND FCHL IN MEN.

| | | | Heterozygosity/ | | | | | |
|---|---|---|---|---|---|---|---|---|
| | | | Rare allele | | | | | |
| | | Distance | frequency in | | | | | LD |
| | | (in | all family | TGs | TGs | FCHL | FCHL | cluster |
| SNP | rs number | bp) | members | HHRR | Gamete | HHRR | Gamete | (I-V) |
| jam1s1 | rs836 | 1361 | 0.41/0.28 | 0.03 | 0.009 | ns | 0.03 | I |
| jam1s2 | rs790056 | 1561 | 0.36/0.24 | ns | 0.03 | ns | ns | II |
| jam1s3 | rs790055 | 25608 | 0.35/0.23 | ns | ns | ns | ns | II |
| jam1s4 | new | 10572 | 0.38/0.26 | 0.06 | 0.04 | ns | ns | I |
| jam1s5 | rs4339888 | 1246 | 0.43/0.31 | 0.02 | 0.003 | ns | 0.09 | I |
| jam1s6 | rs3766383 | 951 | 0.25/0.15 | ns | ns | ns | ns | III |
| usf1s1 | rs3737787 | 1239 | 0.45/0.34 | 0.0009 | 0.00001 | 0.04 | 0.05 | I |
| | | | | (0.01) | (0.008) | (ns) | (ns) | |
| usf1s2 | rs2073658 | 12 | 0.44/0.33 | 0.002 | 0.00006 | 0.04 | ns | I |
| | | | | (0.02) | (0.02) | (ns) | (ns) | |
| usf1s3 | rs2516841 | 17 | 0.40/0.28 | ns | ns | ns | ns | II |
| usf1s4 | rs2073657 | 526 | 0.48/0.41 | ns | ns | ns | ns | IV |
| usf1s5 | rs2516840 | 1443 | 0.41/0.29 | ns | ns | ns | ns | II |
| usf1s6 | rs2073653 | 361 | 0.25/0.14 | ns | 0.08 | ns | ns | III |
| USF1S7 | rs2516839 | 1249 | 0.47/0.39 | ns | 0.04 | ns | ns | IV |
| | | | | (ns) | (ns) | (ns) | (ns) | |
| usf1s8 | rs2516838 | 279 | 0.40/0.28 | 0.01 | 0.05 | ns | ns | V |
| | | | | (0.05) | (0.03) | (ns) | (ns) | |
| usf1s9 | rs1556259 | | 0.23/0.13 | ns | ns | ns | ns | III |

All results represent p-values, ns indicates non-significant, HHRR haplotype-based haplotype relative risk test, and Gamete gamete competition test. LD cluster number in the last column indicates the clusters of SNPs showing strong intermarker LD (p ≦ 0.00002) in the male probands with high TGs (>90^{th} age-sex percentile),i.e. the SNPs carrying the same cluster number are in strong pairwise LD. SNPs indicated in bold were genotyped in the 60 extended FCHL families, and the values in parentheses were obtained for these SNPs in the 42 nuclear FCHL families. All other results were obtained in the 42 nuclear FCHL families.

SUPPLEMENTARY TABLE 3
VARIANTS IDENTIFIED BY SEQUENCING THE USF1 GENE IN THE 31 FCHL
PROBANDS OF THE ORIGINAL LINKAGE STUDY³.

| | | Rare allele | | |
|---|---|---|---|---|
| | | frequencies | Information on LD | |
| Location | rs number | (in 31 samples) | (in 31 samples) | Specifics |
| −2167 | New | 0.02 | | T/C |
| −2022 | New | 0.05 | | A/C |
| −802 | New | 0.03 | | C/G |
| Exon 1 | rs2516837 | 0.44 | In full LD with | Not |
| | | | rs2516839 and | translated |
| | | | rs2774273 | region |
| INTRON 1 = usf1s9 | rs1556259 | 0.19 | | |
| INTRON 1 = usf1s8 | rs2516838 | 0.29 | | |
| Intron 1 | rs1556260 | 0.16 | In full LD with SNPs | |
| | | | in 1125 bp and 1416 bp; | |
| | | | 30/31 samples in | |
| | | | LD with rs1556259 | |
| Intron 1 | rs2774273 | 0.44 | In full LD with | |
| | | | rs2516839 and | |
| | | | rs2516837 | |
| Intron 1/1125 bp | New | 0.16 | In full LD with SNP | C/T |
| | | | 1416 bp; | |
| | | | 30/31 samples in LD | |
| | | | with rs1556259 | |
| Intron 1/1416 bp | New | 0.16 | In full LD with the | A/G |
| | | | SNP in 1125 bp; | |
| | | | 30/31 samples in LD | |
| | | | with rs1556259 | |
| EXON 2 = usf1s7 | rs2516839 | 0.44 | | Not |
| | | | | translated |
| | | | | region |
| INTRON 2 = usf1s6 | rs2073653 | 0.11 | | |
| Intron 3 | rs2073655 | 0.23 | In full LD with | |
| | | | rs2073658 | |
| Intron 5 | rs2774276 | 0.27 | 29/31 in LD with | |
| | | | rs2516840 | |
| Intron 6 | rs2073656 | 0.23 | In full LD with | |
| | | | rs2073658 | |
| INTRON 6 = usf1s5 | rs2516840 | 0.32 | | |
| Intron 6/3411 bp | New | 0.05 | | C/T |
| Intron 6/3519 bp | New | 0.05 | | C/T |
| INTRON 7 = usf1s4 | rs2073657 | 0.47 | | In AluSx |
| INTRON 7 = usf1s3 | rs2516841 | 0.31 | | In AluSx |
| INTRON 7 = usf1s2 | rs2073658 | 0.23 | | |
| Intron 9/4445 bp | New | 0.03 | | A/G |
| EXON 11 = usf1s1 | rs3737787 | 0.24 | | Not |
| | | | | translated |
| | | | | region |

Underlined variants were genotyped in the FCHL families. For these SNPs, the numbers usf1s1-s9, used in the text and Tables 1-3, are also shown; New indicates that the SNP was not found in the SNP databases. The numbering of the new SNPs is based on the genomic sequence of USF1 at the UCSC Genome Browser, July 2003 (refGene_NM_{—}007122).

Next, we genotyped these two associated SNPs, usf1s1 and usf1s2, in the larger study sample of 60 extended FCHL families. Furthermore, 12 additional SNPs were genotyped for the USF1 region (Table 2, FIG. 1). Of the 23 SNPs identified by sequencing, we genotyped all the SNPs that were not in strong LD in 31 probands, excluding six rare SNPs present in three or fewer individuals (Supplementary Table 2). A total of four USF1 SNPs were genotyped in the 60 extended families due to their promising results in the nuclear study sample and/or LD pattern (Table 2). When genotyped in the 60 extended FCHL families, the two individual SNPs, usf1s1 and usf1s2, yielded p-values of 0.0009 and 0.002 in the HHRR test as well as 0.00001 and 0.0006 in the gamete competition test for TGs in men (Table 2). The common allele of both SNPs was more frequently transmitted to the affected individuals in both tests and with both the FCHL and TG traits. The asymptotic p-values of the combined analyses of these two SNPs were 0.00003 in the HHRR and 0.0000009 in the combined gamete competition test for TGs in men (Table 1). The segregating haplotype was 1-1 (1 indicating the common allele). For all TG-affected family members, the combined analysis also produced evidence of association with p-values of 0.05 in the HHRR analysis and 0.00006 in the gamete competition test, again with the segregating haplotype of 1-1 (Table 1).

To confirm that the gamete competition results are indeed significant and not biased by such contributors as sparse data, we calculated empirical p-values for all gamete compete analyses involving multiple SNPs (Table 1) using gene dropping with at least 50,000 simulations (see Methods). The obtained empirical p-values were in very good agreement with the asymptotic p-values of the gamete competition analyses (Table. 1), indicating that the observed results do not represent artifacts of asymptotic approximations with sparse data.

After genotyping a total of 15 SNPs in the USF1 region, we identified a pattern of association and LD reaching at least 46 kb in men with high TGs and extending from the centromeric junctional adhesion molecule 1 (JAM1) gene to the USF1 gene (FIG. 1 and Table 2): in addition to usf1s1 and usf1s2, three other SNPs, jam1s1, jam1s4, and jam1s5, also showed evidence for association in the 42 nuclear FCHL families for high TGs in men (Table 2). These three SNPs were in strong LD with the usf1s1 and usf1s2 (p<0.00002). The LD pattern, tested by the Genepop program, for SNPs in the JAM1-USF1 region is shown in Table 2. In addition to these five SNPs, one SNP (usf1s8) in intron 1 of USF1, showed some evidence for association as well (Table 2). This SNP was not in LD with any of the 14 other SNPs (Table 2).

In all affected family members, using both FCHL and TG traits, the evidence for association was restricted to the usf1 s1 and usf1s2 (Table 1) within the USF1 gene. The rest of the 13 SNPs genotyped for the JAM1-USF1 region did not provide significant evidence for association. However, we observed that two additional USF1 SNPs among those 23 SNPs identified by sequencing, rs2073655 in intron 3 and rs2073656 in intron 6, were also in full LD with the associated usf1s2 in 31 FCHL probands and are likely to extend the FCHL-associated region to intron 3 of USF1. No association was obtained with SNPs residing outside the JAM1-USF1 region (Supplementary Table 1). In conclusion, evidence for association and LD was restricted to a 1239 bp region within the USF1 gene in all affected individuals of FCHL families but extended at least 46 kb within the JAM1-USF1 region in men with high TGs (Tables 2-3, FIG. 1).

The combination of the usf1s1-usf1s2 SNPs, resulting in the significant haplotypes for FCHL and TGs, was also tested with three additional qualitative lipid traits: high apolipoprotein B (apoB), high TC and small low-density lipoprotein (LDL) peak particle size. For apoB, p-values of 0.00003 and 0.0007 were obtained for all affected individuals and for affected men for the susceptibility haplotype 1-1 in the gamete competition analysis. For TC, the p-values were 0.0001 and 0.007; and for LDL peak particle size, 0.002 and 0.01, respectively. These results together with the results obtained for FCHL suggest that the underlying gene is not affecting TGs alone but also the complex FCHL phenotype.

### EXAMPLE 3

### Haplotype Analyses of the JAM1-USF1 Gene Region

Using the HBAT program we obtained evidence for shared haplotypes in the region of usf1s1 and usf1s2 (Table 3). This observation was supported by multipoint HHRR analyses (Table 3). For the haplotype 1-1 (1 indicating the common allele) a p-value of 0.0007 was obtained using the option.

TABLE 3
HAPLOTYPE ANALYSES IN TG-AFFECTED MEN USING THE HBAT
PROGRAM (THE MULTILOCUS GENO-PDT AND MULTI-HHRR
RESULTS ARE GIVEN BELOW FOR COMPARISON).

| | Haplotype of SNPs: | Haplotype of SNPs: | Haplotype of SNPs: |
|---|---|---|---|
| Test | Jam1s4-6 - usf1s1-2 | usf1s1-2 | usf1s1-5 |
| HBAT-o | P = 0.03 | P = 0.0007 | P = ns (0.07) |
| | (haplotype 1-1-1-1-1) | (haplotype 1-1) | (haplotype 1-1-1-1-1) |
| | | P = 0.004 for the | |
| | | protective haplotype 2-2, | |
| | | significantly less | |
| | | transmitted to the | |
| | | affected subjects | |
| HBAT-e | P = 0.009 | P = 0.02 | P = ns (0.2) |
| | (haplotype 1-1-1-1-1) | (haplotype 1-1) | (haplotype 1-1-1-1-1) |
| Multi- | P = 0.02 | P = 0.002 | P = ns (0.7) |
| locus | | | |
| geno- | | | |
| PDT | | | |
| Multi- | P = 0.0002 | P = 0.00003 | P = 0.04 |
| HHRR | | | |

The inter-SNP distances and corresponding rs numbers for the SNPs jam1s4-s6 and usf1s1-s5 are shown in Table 2; 1 indicates the common allele; and ns non-significant. The p-value of the HBAT program indicates the probability that the particular haplotype is transmitted to the affected individuals using the option -o (optimize offset) or option -e (empirical test). Multilocus geno-PDT indicates a genotype-based association test forgeneral pedigrees. The multi-HHRR analysis is testing the hypothesis of homogeneity of marker allele distributions between transmitted and non-transmitted alleles of the SNPs.

This option measures not only preferential transmission of the susceptibility haplotype to affected but also less preferential transmissions to unaffected, making it useful here since in these extended families the unaffecteds also contain important information. The results of the HBAT-e option, a test of association given linkage, are also shown in Table 3. Since this test statistics implicitly conditions on linkage information, it is less powerful and leads to reduced p-values. However, this test together with the results of the HHRR analyses allow us to conclude that the 1-1 haplotype is associated with the phenotype (Table 3). Furthermore, haplotype 2-2 was significantly less transmitted to the affected subjects (p=0.004), suggesting a protective role for this allele. These results were further supported by a genotype-based association test for general pedigrees, the genotype-PDT, which provided evidence for association (Table 3), as well as by the gamete competition analyses (Table 1), where the same haplotype 1-1 was segregating to the affected individuals with both FCHL and TG traits.

### EXAMPLE 4

### Expression Profiles of Fat Biopsies and Initial Functional Analysis

We investigated Whether the gene expression profiles of fat biopsies from six affected FCHL family members carrying the susceptibility haplotype 1-1, constructed by the SNPs usf1s1 and usf1s2, revealed differences when compared to four affected FCHL family members homozygous for the putative protective haplotype, 2-2 (see above), using the Affymetrix, HGU133A probe array. We also specifically investigated whether USF1 is expressed in fat tissue because it is not sufficiently represented on the Affymetrix HGU133A chip. Using RT-PCR the USF1 was found to be expressed in the fat biopsy samples (data not shown). Quantitative real-time PCR was also performed to determine the relative expression levels of USF1 in adipose tissue in the affected FCHL family members carrying the risk haplotype and affected members not carrying the risk haplotype. No detectable differences in USF1 expression levels could be observed, suggesting that the potential functional significance of the FCHL associated allele of the USF1 is not delivered via a direct effect on the steady state transcript level in adipose tissue.

Due to the limited number of samples available, statistical power to detect differences in gene expression between the haplotype groups was not considered sufficient. As an alternative, we therefore defined cut-off thresholds (see Methods) to discriminate between significant differences and differences attributable to technical or biological noise in the experimental procedures. Using these criteria, we identified 25 genes that appeared up-regulated and 73 genes down-regulated in the susceptibility haplotype carriers (the complete lists will be available at our website, while the raw data can be accessed through the Gene Expression Omnibus at NCBI using the GEO accession GSE590). To lend biological relevance to these findings, lists of differentially expressed genes were examined for over-representation of functional classes, as defined by the gene ontology (GO) consortium, using the Expression Analysis Systematic Explorer (EASE) tool. Only three classes were found to be statistically significantly over-represented among the up-regulated genes (FIG. 2), primarily implicating genes involved in fat metabolism. Among the down-regulated genes, a prominent down-regulation of immune-response genes was observed (FIG. 2). The complete results from the EASE analysis, including the corresponding EASE scores (p-values) and lists of genes in the significant (=p-value<0.05) functional categories, are given in the Supplementary Table 3a-b.

Next we investigated the genomic sequence flanking the haplotype 1-1, and identified a 60-bp sequence element found in 91 human genes as follows: The SNP usf1s2, forming part of the haplotype 1-1, resides adjacent (8 bp) to a 306-bp AluSx repeat. Two parts (2-61 bp and 137-196 bp) of this AluSx repeat show sequence similarity with the mouse B1 repeat (FIG. 3a). When blasted against the mouse sequence databases, these two parts of the AluSx sequence identify numerous mouse ESTs, due to the B1 element located in the untranslated region of the mouse mRNA. When blasted against human sequence databases, 91 human genes, including USF1, have this 60-bp part of AluSx either on the coding strand (43 genes) or on the opposite strand (48 genes). The 60-bp part is highly conserved from human to worm since it was found in pufferfish and Caenorhabditis elegans but not in Drosophila melanogaster or in Saccharomyces cerevisiae. A complete list of the 91 human genes as well as their individual p-values and identity percentages (between 83-98%) are given in Supplementary Table 4. Analysis of domain annotation of the 91 genes indicates enrichment of domains involved in protein modification (n=16) and domains related to nucleic acids (n=35). This observation was also supported by the available annotations about biological process, where majority of the genes were involved in nucleic acid metabolism (n=18), as well as in transcription and signal transduction (n=33).

To obtain some evidence for the functional significance of this conserved 60-bp DNA element, we produced a 268-bp long construct containing the critical 60-bp sequence as well as the usf1 s2 SNP region and tested its regulatory function in vitro using the SEAP reporter system (FIG. 3b). The genomic DNAs from one homozygous susceptibility carrier (haplotype 1-1) and one homozygous non-carrier (2-2) were cloned in front of the SEAP reporter gene in two orientations. The effect on the transcription of the reporter gene was implicated in the forward orientation in both constructs, whereas the reverse orientation resulted in the transcription efficiency comparable to the negative control (FIG. 3b).

SUPPLEMENTARY TABLE 4A
RESULTS FROM ANALYSIS OF LISTS WITH DIFFERENTIALLY EXPRESSED
GENES BETWEEN THE HAPLOTYPE CARRIERS AND NON-CARRIERS
FOR OVER-REPRESENTATION OF FUNCTIONAL CATEGORIES USING
THE EASE TOOL²⁷. THIS SUPPLEMENTARY TABLE 4A-B WILL
BE SHOWN AT OUR WEB SITE. PLEASE SEE FIG. 2 FOR THE
GRAPHICAL DISTRIBUTION OF THESE GENES ACCORDING TO THE
FUNCTIONAL CATEGORY.

| | | | | | |
|---|---|---|---|---|---|
| | | | | | EASE |
| Functional category¹ | LH | LT | PH | PT | score (p-value) |

UP-REGULATED GENES

| | | | | | |
|---|---|---|---|---|---|
| fatty acid metabolism | 3 | 16 | 90 | 7689 | 0.0129 |
| lipid metabolism | 4 | 16 | 359 | 7689 | 0.0302 |
| macromolecule catabolism | 4 | 16 | 395 | 7689 | 0.0386 |
| carboxylic acid metabolism | 3 | 16 | 230 | 7689 | 0.0724 |
| organic acid metabolism | 3 | 16 | 232 | 7689 | 0.0735 |
| cell motility | 3 | 16 | 253 | 7689 | 0.0855 |
| Catabolism | 4 | 16 | 554 | 7689 | 0.0885 |
| proteolysis and peptidolysis | 3 | 16 | 368 | 7689 | 0.159 |
| protein catabolism | 3 | 16 | 374 | 7689 | 0.164 |
| Metabolism | 11 | 16 | 4163 | 7689 | 0.239 |
| cell proliferation | 3 | 16 | 782 | 7689 | 0.46 |
| Physiological processes | 14 | 16 | 6379 | 7689 | 0.516 |
| cell growth and/or maintenance | 6 | 16 | 2389 | 7689 | 0.521 |
| protein metabolism | 4 | 16 | 1512 | 7689 | 0.589 |
| cellular process | 9 | 16 | 4297 | 7689 | 0.679 |
| cell communication | 4 | 16 | 2238 | 7689 | 0.858 |
| nucleobase, nucleoside, nucleotide and nucleic | 3 | 16 | 1716 | 7689 | 0.88 |
| acid metabolism | | | | | |

Down-regulated genes

| | | | | | |
|---|---|---|---|---|---|
| immune response | 16 | 60 | 560 | 7689 | 0.0000141 |
| response to pest/pathogen/parasite | 13 | 60 | 379 | 7689 | 0.0000236 |
| response to biotic stimulus | 17 | 60 | 674 | 7689 | 0.0000309 |
| defense response | 16 | 60 | 616 | 7689 | 0.0000435 |
| response to wounding | 9 | 60 | 222 | 7689 | 0.000265 |
| response to stress | 14 | 60 | 632 | 7689 | 0.000811 |
| inflammatory response | 7 | 60 | 149 | 7689 | 0.000926 |
| innate immune response | 7 | 60 | 151 | 7689 | 0.000992 |
| response to external stimulus | 17 | 60 | 992 | 7689 | 0.00256 |
| Catabolism | 12 | 60 | 554 | 7689 | 0.00288 |
| colony morphology | 3 | 60 | 26 | 7689 | 0.0167 |
| invasive growth | 3 | 60 | 26 | 7689 | 0.0167 |
| cytosolic calcium ion concentration elevation | 3 | 60 | 32 | 7689 | 0.0248 |
| cellular morphogenesis | 3 | 60 | 34 | 7689 | 0.0277 |
| cell adhesion | 8 | 60 | 390 | 7689 | 0.0287 |
| macromolecule catabolism | 8 | 60 | 395 | 7689 | 0.0305 |
| lipid catabolism | 3 | 60 | 50 | 7689 | 0.0561 |
| proteolysis and peptidolysis | 7 | 60 | 368 | 7689 | 0.0617 |
| protein catabolism | 7 | 60 | 374 | 7689 | 0.0657 |
| G-protein signaling, coupled to IP3 second | 3 | 60 | 66 | 7689 | 0.0909 |
| messenger (phospholipase C activating) | | | | | |
| Endocytosis | 3 | 60 | 72 | 7689 | 0.105 |
| cellular defense response | 3 | 60 | 77 | 7689 | 0.118 |
| lipid metabolism | 6 | 60 | 359 | 7689 | 0.14 |
| Chemotaxis | 3 | 60 | 90 | 7689 | 0.151 |
| Taxis | 3 | 60 | 90 | 7689 | 0.151 |
| Antimicrobial humoral response | 3 | 60 | 92 | 7689 | 0.157 |
| humoral defense mechanism (sensu | 3 | 60 | 92 | 7689 | 0.157 |
| Invertebrata) | | | | | |
| antimicrobial humoral response (sensu | 3 | 60 | 92 | 7689 | 0.157 |
| Invertebrata) | | | | | |
| vesicle-mediated transport | 4 | 60 | 214 | 7689 | 0.226 |
| cell-cell adhesion | 3 | 60 | 136 | 7689 | 0.28 |
| response to chemical substance | 3 | 60 | 141 | 7689 | 0.295 |
| alcohol metabolism | 3 | 60 | 149 | 7689 | 0.317 |
| humoral immune response | 3 | 60 | 152 | 7689 | 0.326 |
| cell surface receptor linked signal transduction | 8 | 60 | 739 | 7689 | 0.338 |
| cell communication | 20 | 60 | 2238 | 7689 | 0.345 |
| signal transduction | 16 | 60 | 1785 | 7689 | 0.392 |
| cell death | 4 | 60 | 313 | 7689 | 0.433 |
| Death | 4 | 60 | 316 | 7689 | 0.439 |
| Physiological processes | 51 | 60 | 6379 | 7689 | 0.439 |
| G-protein coupled receptor protein signaling | 5 | 60 | 457 | 7689 | 0.469 |
| pathway | | | | | |
| metal ion transport | 3 | 60 | 216 | 7689 | 0.497 |
| protein metabolism | 13 | 60 | 1512 | 7689 | 0.5 |
| phosphate metabolism | 5 | 60 | 487 | 7689 | 0.519 |
| phosphorus metabolism | 5 | 60 | 487 | 7689 | 0.519 |
| Transport | 10 | 60 | 1144 | 7689 | 0.524 |
| Development | 10 | 60 | 1165 | 7689 | 0.547 |
| cellular process | 34 | 60 | 4297 | 7689 | 0.551 |
| morphogenesis | 6 | 60 | 669 | 7689 | 0.592 |
| Carbohydrate metabolism | 3 | 60 | 261 | 7689 | 0.6 |
| ion transport | 4 | 60 | 410 | 7689 | 0.616 |
| cation transport | 3 | 60 | 288 | 7689 | 0.655 |
| Apoptosis | 3 | 60 | 289 | 7689 | 0.656 |
| Programmed cell death | 3 | 60 | 290 | 7689 | 0.658 |
| cell organization and biogenesis | 4 | 60 | 437 | 7689 | 0.66 |
| intracellular signaling cascade | 5 | 60 | 596 | 7689 | 0.681 |
| cell growth and/or maintenance | 18 | 60 | 2389 | 7689 | 0.693 |
| Metabolism | 31 | 60 | 4163 | 7689 | 0.74 |
| protein amino acid phosphorylation | 3 | 60 | 365 | 7689 | 0.778 |
| response to abiotic stimulus | 3 | 60 | 389 | 7689 | 0.807 |
| phosphorylation | 3 | 60 | 393 | 7689 | 0.812 |
| organogenesis | 4 | 60 | 637 | 7689 | 0.878 |
| protein modification | 4 | 60 | 682 | 7689 | 0.905 |
| cell proliferation | 3 | 60 | 782 | 7689 | 0.987 |
| regulation of transcription, DNA-dependent | 3 | 60 | 974 | 7689 | 0.997 |
| regulation of transcription | 3 | 60 | 979 | 7689 | 0.997 |
| transcription, DNA-dependent | 3 | 60 | 1085 | 7689 | 0.999 |
| Transcription | 3 | 60 | 1112 | 7689 | 0.999 |
| nucleobase, nucleoside, nucleotide and nucleic | 5 | 60 | 1716 | 7689 | 1 |
| acid metabolism | | | | | |

¹According to the gene ontology (GO) classification biological process⁴¹.
Abbreviations:
LH—list hits,
LT—list total,
PH—population hits,
PT—population total, and
EASE—Expression Analysis Systematic Explorer⁴².
The complete lists of genes in each functional category will be presented at our web site.

SUPPLEMENTARY TABLE 4b
Lists of genes in the significant (= EASE p-value < 0.05) functional categories in Table 3a above. This
supplementary Table 4a-b will be shown at our web site.

| | | | | |
|---|---|---|---|---|
| | LOCUS- | | | |
| UNIQID | LINK | GENENAME | CLASSIFICATION | LOCUSLINK CLASSIFICATIONS |

Up-regulated genes
Fatty acid metabolism

| | | | | |
|---|---|---|---|---|
| 200832_s_at | 6319 | stearoyl-coA | biological_process | endoplasmic reticulum; fatty acid biosynthesis; integral to membrane; |
| | | desaturase | | iron ion binding; oxidoreductase activity; stearoyl-CoA |
| | | (delta-9- | | desaturase activity |
| | | desaturase) | | |
| 206930_at | 10249 | glycine-N- | biological_process | acyl-CoA metabolism; acyltransferase activity; mitochondrion; |
| | | acyltransferase | | response to toxin |
| 209600_s_at | 51 | acyl-Coenzyme | biological_process | acyl-CoA oxidase activity; electron donor activity; electron transport; |
| | | A oxidase 1, | | energy pathways; fatty acid beta-oxidation; oxidoreductase activity; |
| | | palmitoyl | | peroxisome; prostaglandin metabolism |

Lipid metabolism

| | | | | |
|---|---|---|---|---|
| 200832_s_at | 6319 | stearoyl-CoA | biological_process | endoplasmic reticulum; fatty acid biosynthesis; integral to membrane; |
| | | desaturase | | iron ion binding; oxidoreductase activity; stearoyl-CoA |
| | | (delta-9- | | desaturase activity |
| | | desaturase) | | |
| 202118_s_at | 8895 | copine III | biological_process | calcium-dependent phospholipid binding; cell adhesion |
| | | | | molecule activity; |
| | | | | lipid metabolism; transporter activity; vesicle-mediated transport |
| 206930_at | 10249 | glycine-N- | biological_process | acyl-CoA metabolism; acyltransferase activity; mitochondrion; |
| | | acyltransferase | | response to toxin |
| 209600_s_at | 51 | acyl-Coenzyme | biological_process | acyl-CoA oxidase activity; electron donor activity; electron transport; |
| | | A oxidase 1, | | energy pathways; fatty acid beta-oxidation; oxidoreductase activity; |
| | | palmitoyl | | peroxisome; prostaglandin metabolism |

Macromolecule catabolism

| | | | | |
|---|---|---|---|---|
| 202581_at | 3304 | heat shock 70 kDa | biological_process | ATP binding; cytoplasm; heat shock protein activity; |
| | | protein 1B | | mRNA catabolism; nucleus |
| 204844_at | 2028 | glutamyl | biological_process | cell proliferation; cell-cell signaling; glutamyl aminopeptidase activity; |
| | | aminopeptidase | | hydrolase activity; integral to plasma membrane; membrane alanyl |
| | | (aminopeptidase | | aminopeptidase activity; metallopeptidase activity; proteolysis and |
| | | A) | | peptidolysis; zinc ion binding |
| 209788_s_at | 51752 | type 1 tumor | biological_process | aminopeptidase activity; membrane alanyl aminopeptidase activity; |
| | | necrosis factor | | metallopeptidase activity; proteolysis and peptidolysis; zinc ion binding |
| | | receptor shedding | | |
| | | aminopeptidase | | |
| | | regulator | | |
| 215271_at | 63923 | tenascin N | biological_process | carboxypeptidase A activity; cell growth; cell, migration; |
| | | | | cellular_component unknown; molecular_function unknown; |
| | | | | proteolysis and peptidolysis |

Down-regulated genes
Immune response

| | | | | |
|---|---|---|---|---|
| 201422_at | 10437 | interferon, gamma- | biological_process | extracellular; immune response; lysosome; oxidoreductase |
| | | inducible protein 30 | | activity |
| 201952_at | 214 | activated leukocyte cell | biological_process | antimicrobial humoral response (sensu Invertebrata); cell |
| | | adhesion molecule | | adhesion; cell adhesion molecule activity; integral to plasma |
| | | | | membrane; membrane fraction; receptor binding; signal |
| | | | | transduction |
| 202803_s_at | 3689 | integrin, beta 2 (antigen | biological_process | antimicrobial humoral response (sensu Invertebrata); cell |
| | | CD18 (p95), lymphocyte | | adhesion; cell adhesion receptor activity; integrin complex; |
| | | function-associated antigen | | integrin-mediated signaling pathway |
| | | 1; macrophage antigen 1 | | |
| | | (mac-1) beta subunit) | | |
| 202901_x_at | 1520 | cathepsin S | biological_process | cathepsin S activity; hydrolase activity; immune response; |
| | | | | lysosome; proteolysis and peptidolysis |
| 203104_at | 1436 | colony stimulating factor 1 | biological_process | ATP binding; antimicrobial humoral response (sensu |
| | | receptor, formerly | | Invertebrata); cell proliferation; development; integral to plasma |
| | | McDonough feline sarcoma | | membrane; macrophage colony stimulating factor receptor |
| | | viral (v-fms) oncogene | | activity; protein amino acid phosphorylation; receptor activity; |
| | | homolog | | signal transduction; transferase activity; transmembrane |
| | | | | receptor protein tyrosine kinase signaling pathway |
| 203382_s_at | 348 | apolipoprotein E | biological_process | cholesterol metabolism; circulation; development; heparin |
| | | | | binding; immune response; lipid binding; lipid metabolism; lipid |
| | | | | transport; lipid transporter activity; receptor binding |
| 203650_at | 10544 | protein C receptor, | biological_process | blood coagulation; inflammatory response; integral to plasma |
| | | endothelial (EPCR) | | membrane; receptor activity |
| 204122_at | 7305 | TYRO protein tyrosine | biological_process | cellular defense response; integral to plasma membrane; |
| | | kinase binding protein | | intracellular signaling cascade; receptor signaling protein |
| | | | | activity |
| 204446_s_at | 240 | arachidonate 5- | biological_process | arachidonate 5-lipoxygenase activity; electron transport; |
| | | lipoxygenase | | inflammatory response; iron ion binding; leukotriene |
| | | | | biosynthesis; lipoxygenase activity; oxidoreductase activity |
| 205098_at | 1230 | chemokine (C—C motif) | biological_process | C—C chemokine receptor activity; G-protein signaling, coupled to |
| | | receptor 1 | | cyclic nucleotide second messenger; cell adhesion; cell-cell |
| | | | | signaling; chemotaxis; cytosolic calcium ion concentration |
| | | | | elevation; immune response; inflammatory response; integral to |
| | | | | plasma membrane; invasive growth; rhodopsin-like receptor |
| | | | | activity |
| 206214_at | 7941 | phospholipase A2, group | biological_process | 2-acetyl-1-alkylglycerophosphocholine esterase activity; 2- |
| | | VII (platelet-activating | | acetyl-1-alkylglycerophosphocholine esterase complex; |
| | | factor acetylhydrolase, | | extracellular; hydrolase activity; inflammatory response; lipid |
| | | plasma) | | catabolism; phospholipid binding |
| 209906_at | 719 | complement component 3a | biological_process | C3a anaphylatoxin receptor activity; G-protein coupled receptor |
| | | receptor 1 | | protein signaling pathway; cell motility; cellular defense |
| | | | | response; chemotaxis; circulation; complement component C3a |
| | | | | receptor activity; cytosolic calcium ion concentration elevation; |
| | | | | inflammatory response; integral to plasma membrane; |
| | | | | phosphatidylinositol-4,5-bisphosphate hydrolysis; rhodopsin-like |
| | | | | receptor activity; smooth muscle contraction |
| 211530_x_at | 3135 | HLA-G histocompatibility | biological_process | MHC class I receptor activity; antigen presentation, endogenous |
| | | antigen, class I, G | | antigen; antigen processing, endogenous antigen via MHC |
| | | | | class I; cellular defense response; integral to membrane; |
| | | | | perception of pest/pathogen/parasite |
| 211799_x_at | 3107 | major histocompatibility | biological_process | MHC class II receptor activity; class I major histocompatibility |
| | | complex, class I, C | | complex antigen; immune response; integral to membrane |
| 213975_s_at | 4069 | lysozyme (renal | biological_process | carbohydrate metabolism; cell wall catabolism; cytolysis; |
| | | amyloidosis) | | extracellular space; hydrolase activity, acting on glycosyl bonds; |
| | | | | inflammatory response; lysin activity; lysozyme activity |
| 217028_at | 7852 | chemokine (C—X—C | biological_process | C—C chemokine receptor activity; C—X—C chemokine receptor |
| | | motif) receptor 4 | | activity; G-protein coupled receptor protein signaling pathway; |
| | | | | activation of MAPK; apoptosis; chemotaxis; coreceptor activity; |
| | | | | cytoplasm; cytosolic calcium ion concentration elevation; |
| | | | | histogenesis and organogenesis; immune response; |
| | | | | inflammatory response; integral to plasma membrane; invasive |
| | | | | growth; neurogenesis; pathogenesis; response to viruses; |
| | | | | rhodopsin-like receptor activity |

Response to pest/pathogen/parasite

| | | | | |
|---|---|---|---|---|
| 201850_at | 822 | capping protein (actin | biological_process | F-actin capping protein complex; actin binding; barbed-end |
| | | filament), gelsolin-like | | actin capping activity; nucleus; protein complex assembly; |
| | | | | response to pest/pathogen/parasite |
| 201952_at | 214 | activated leukocyte cell | biological_process | antimicrobial humoral response (sensu Invertebrate); cell |
| | | adhesion molecule | | adhesion; cell adhesion molecule activity; integral to plasma |
| | | | | membrane; membrane fraction; receptor binding; signal |
| | | | | transduction |
| 202803_s_at | 3689 | integrin, beta 2 (antigen | biological_process | antimicrobial humoral response (sensu Invertebrata); cell |
| | | CD18 (p95), lymphocyte | | adhesion; cell adhesion receptor activity; integrin complex; |
| | | function-associated antigen | | integrin-mediated signaling pathway |
| | | 1; macrophage antigen 1 | | |
| | | (mac-1) beta subunit) | | |
| 203104_at | 1436 | colony stimulating factor 1 | biological_process | ATP binding; antimicrobial humoral response (sensu |
| | | receptor, formerly | | Invertebrata); cell proliferation; development; integral to plasma |
| | | McDonough feline sarcoma | | membrane; macrophage colony stimulating factor receptor |
| | | viral (v-fms) oncogene | | activity; protein amino acid phosphorylation; receptor activity; |
| | | homolog | | signal transduction; transferase activity; transmembrane |
| | | | | receptor protein tyrosine kinase signaling pathway |
| 203650_at | 10544 | protein C receptor, | biological_process | blood coagulation; inflammatory response; integral to plasma |
| | | endothelial (EPCR) | | membrane; receptor activity |
| 204122_at | 7305 | TYRO protein tyrosine | biological_process | cellular defense response; integral to plasma membrane; |
| | | kinase binding protein | | intracellular signaling cascade; receptor signaling protein |
| | | | | activity |
| 204446_s_at | 240 | arachidonate 5- | biological_process | arachidonate 5-lipoxygenase activity; electron transport; |
| | | lipoxygenase | | inflammatory response; iron ion binding; leukotriene |
| | | | | biosynthesis; lipoxygenase activity; oxidoreductase activity |
| 205098_at | 1230 | chemokine (C—C motif) | biological_process | C—C chemokine receptor activity; G-protein signaling, coupled to |
| | | receptor 1 | | cyclic nucleotide second messenger; cell adhesion; cell-cell |
| | | | | signaling; chemotaxis; cytosolic calcium ion concentration |
| | | | | elevation; immune response; inflammatory response; integral to |
| | | | | plasma membrane; invasive growth; rhodopsin-like receptor |
| | | | | activity |
| 206214_at | 7941 | phospholipase A2, group | biological_process | 2-acetyl-1-alkylglycerophosphocholine esterase activity; 2- |
| | | VII (platelet-activating | | acetyl-1-alkylglycerophosphocholine esterase complex; |
| | | factor acetylhydrolase, | | extracellular; hydrolase activity; inflammatory response; lipid |
| | | plasma) | | catabolism; phospholipid binding |
| 209906_at | 719 | complement component 3a | biological_process | C3a anaphylatoxin receptor activity; G-protein coupled receptor |
| | | receptor 1 | | protein signaling pathway; cell motility; cellular defense |
| | | | | response; chemotaxis; circulation; complement component C3a |
| | | | | receptor activity; cytosolic calcium ion concentration elevation; |
| | | | | inflammatory response; integral to plasma membrane; |
| | | | | phosphatidylinositol-4,5-bisphosphate hydrolysis; rhodopsin-like |
| | | | | receptor activity; smooth muscle contraction |
| 211530_x_at | 3135 | HLA-G histocompatibility | biological_process | MHC class I receptor activity; antigen presentation, endogenous |
| | | antigen, class I, G | | antigen; antigen processing, endogenous antigen via MHC |
| | | | | class I; cellular defense response; integral to membrane; |
| | | | | perception of pest/pathogen/parasite |
| 213975_s_at | 4069 | lysozyme (renal | biological_process | carbohydrate metabolism; cell wall catabolism; cytolysis; |
| | | amyloidosis) | | extracellular space; hydrolase activity, acting on glycosyl bonds; |
| | | | | inflammatory response; lysin activity; lysozyme activity |
| 217028_at | 7852 | chemokine (C—X—C | biological_process | C—C chemokine receptor activity; C—X—C chemokine receptor |
| | | motif) receptor 4 | | activity; G-protein coupled receptor protein signaling pathway; |
| | | | | activation of MAPK; apoptosis; chemotaxis; coreceptor activity; |
| | | | | cytoplasm; cytosolic calcium ion concentration elevation; |
| | | | | histogenesis and organogenesis; immune response; |
| | | | | inflammatory response; integral to plasma membrane; invasive |
| | | | | growth; neurogenesis; pathogenesis; response to viruses; |
| | | | | rhodopsin-like receptor activity |

Response to biotic stimulus

| | | | | |
|---|---|---|---|---|
| 201422_at | 10437 | interferon, gamma- | biological_process | extracellular; immune response; lysosome; oxidoreductase activity |
| | | inducible protein 30 | | |
| 201850_at | 822 | capping protein (actin | biological_process | F-actin capping protein complex; actin binding; barbed-end actin |
| | | filament), gelsolin-like | | capping activity; nucleus; protein complex assembly; response to |
| | | | | pest/pathogen/parasite |
| 201952_at | 214 | activated leukocyte cell | biological_process | antimicrobial humoral response (sensu Invertebrata); cell |
| | | adhesion molecule | | adhesion; cell adhesion molecule activity; integral to plasma |
| | | | | membrane; membrane fraction; receptor binding; signal |
| | | | | transduction |
| 202803_s_at | 3689 | integrin, beta 2 (antigen | biological_process | antimicrobial humoral response (sensu Invertebrata); cell |
| | | CD18 (p95), lymphocyte | | adhesion; cell adhesion receptor activity; integrin complex; |
| | | function-associated antigen | | integrin-mediated signaling pathway |
| | | 1; macrophage antigen 1 | | |
| | | (mac-1) (beta subunit) | | |
| 202901_x_at | 1520 | cathepsin S | biological_process | cathepsin S activity; hydrolase activity; immune response; |
| | | | | lysosome; proteolysis and peptidolysis |
| 203104_at | 1436 | colony stimulating factor 1 | biological_process | ATP binding; antimicrobial humoral response (sensu Invertebrata); |
| | | receptor, formerly | | cell proliferation; development; integral to plasma membrane; |
| | | McDonough feline sarcoma | | macrophage colony stimulating factor receptor activity; protein |
| | | viral (v-fms) oncogene | | amino acid phosphorylation; receptor activity; signal transduction; |
| | | homolog | | transferase activity; transmembrane receptor protein tyrosine |
| | | | | kinase signaling pathway |
| 203382_s_at | 348 | apolipoprotein E | biological_process | cholesterol metabolism; circulation; development; heparin binding; |
| | | | | immune response; lipid binding; lipid metabolism; lipid transport; |
| | | | | lipid transporter activity; receptor binding |
| 203650_at | 10544 | protein C receptor, | biological_process | blood coagulation; inflammatory response; integral to plasma |
| | | endothelial (EPCR) | | membrane; receptor activity |
| 204122_at | 7305 | TYRO protein tyrosine | biological_process | cellular defense response; integral to plasma membrane; |
| | | kinase binding protein | | intracellular signaling cascade; receptor signaling protein activity |
| 04446_s_at | 240 | arachidonate 5- | biological_process | arachidonate 5-lipoxygenase activity; electron transport; |
| | | lipoxygenase | | inflammatory response; iron ion binding; leukotriene biosynthesis; |
| | | | | lipoxygenase activity; oxidoreductase activity |
| 205098_at | 1230 | chemokine (C—C motif) | biological_process | C—C chemokine receptor activity; G-protein signaling, coupled to |
| | | receptor 1 | | cyclic nucleotide second messenger; cell adhesion; cell-cell |
| | | | | signaling; chemotaxis; cytosolic calcium ion concentration |
| | | | | elevation; immune response; inflammatory response; integral to |
| | | | | plasma membrane; invasive growth; rhodopsin-like receptor |
| | | | | activity |
| 206214_at | 7941 | phospholipase A2, group | biological_process | 2-acetyl-1-alkylglycerophosphocholine esterase activity; 2-acetyl- |
| | | VII (platelet-activating | | 1-alkylglycerophosphocholine esterase complex; extracellular; |
| | | factor acetylhydrolase, | | hydrolase activity; inflammatory response; lipid catabolism; |
| | | plasma) | | phospholipid binding |
| 209906_at | 719 | complement component 3a | biological_process | C3a anaphylatoxin receptor activity; G-protein coupled receptor |
| | | receptor 1 | | protein signaling pathway; cell motility; cellular defense response; |
| | | | | chemotaxis; circulation; complement component C3a receptor |
| | | | | activity; cytosolic calcium ion concentration elevation; |
| | | | | inflammatory response; integral to plasma membrane; |
| | | | | phosphatidylinositol-4,5-bisphosphate hydrolysis; rhodopsin-like |
| | | | | receptor activity; smooth muscle contraction |
| 211530_x_at | 3135 | HLA-G histocompatibility | biological_process | MHC class I receptor activity; antigen presentation, endogenous |
| | | antigen, class I, G | | antigen; antigen processing, endogenous antigen via MHC class I; |
| | | | | cellular defense response; integral to membrane; perception of |
| | | | | pest/pathogen/parasite |
| 211799_x_at | 3107 | major histocompatibility | biological_process | MHC class II receptor activity; class I major histocompatibility |
| | | complex, class I, C | | complex antigen; immune response; integral to membrane |
| 213975_s_at | 4069 | lysozyme (renal | biological_process | carbohydrate metabolism; cell wall catabolism; cytolysis; |
| | | amyloidosis) | | extracellular space; hydrolase activity, acting on glycosyl bonds; |
| | | | | inflammatory response; lysin activity; lysozyme activity |
| 217028_at | 7852 | chemokine (C—X—C | biological_process | C—C chemokine receptor activity; C—X—C chemokine receptor |
| | | motif) receptor 4 | | activity; G-protein coupled receptor protein signaling pathway; |
| | | | | activation of MAPK; apoptosis; chemotaxis; coreceptor activity; |
| | | | | cytoplasm; cytosolic calcium ion concentration elevation; |
| | | | | histogenesis and organogenesis; immune response; inflammatory |
| | | | | response; integral to plasma membrane; invasive growth; |
| | | | | neurogenesis; pathogenesis; response to viruses; rhodopsin-like |
| | | | | receptor activity |

Defense response

| | | | | |
|---|---|---|---|---|
| 201422_at | 10437 | interferon, gamma- | biological_process | extracellular; immune response; lysosome; oxidoreductase |
| | | inducible protein 30 | | activity |
| 201952_at | 214 | activated leukocyte cell | biological_process | antimicrobial humoral response (sensu Invertebrata); cell |
| | | adhesion molecule | | adhesion; cell adhesion molecule activity; integral to plasma |
| | | | | membrane; membrane fraction; receptor binding; signal |
| | | | | transduction |
| 202803_s_at | 3689 | integrin, beta 2 (antigen | biological_process | antimicrobial humoral response (sensu Invertebrata); cell |
| | | CD18 (p95), lymphocyte | | adhesion; cell adhesion receptor activity; integrin complex; |
| | | function-associated antigen | | integrin-mediated signaling pathway |
| | | 1; macrophage antigen 1 | | |
| | | (mac-1) (beta subunit) | | |
| 202901_x_at | 1520 | cathepsin S | biological_process | cathepsin S activity; hydrolase activity; immune response; |
| | | | | lysosome; proteolysis and peptidolysis |
| 203104_at | 1436 | colony stimulating factor 1 | biological_process | ATP binding; antimicrobial humoral response (sensu |
| | | receptor, formerly | | Invertebrata); cell proliferation; development; integral to plasma |
| | | McDonough feline sarcoma | | membrane; macrophage colony stimulating factor receptor |
| | | viral (v-fms) oncogene | | activity; protein amino acid phosphorylation; receptor activity; |
| | | homolog | | signal transduction; transferase activity; transmembrane |
| | | | | receptor protein tyrosine kinase signaling pathway |
| 203382_s_at | 348 | apolipoprotein E | biological_process | cholesterol metabolism; circulation; development; heparin |
| | | | | binding; immune response; lipid binding; lipid metabolism; lipid |
| | | | | transport; lipid transporter activity; receptor binding |
| 203650_at | 10544 | protein C receptor, | biological_process | blood coagulation; inflammatory response; integral to plasma |
| | | endothelial (EPCR) | | membrane; receptor activity |
| 204122_at | 7305 | TYRO protein tyrosine | biological_process | cellular defense response; integral to plasma membrane; |
| | | kinase binding protein | | intracellular signaling cascade; receptor signaling protein |
| | | | | activity |
| 204446_s_at | 240 | arachidonate 5- | biological_process | arachidonate 5-lipoxygenase activity; electron transport; |
| | | lipoxygenase | | inflammatory response; iron ion binding; leukotriene |
| | | | | biosynthesis; lipoxygenase activity; oxidoreductase activity |
| 205098_at | 1230 | chemokine (C—C motif) | biological_process | C—C chemokine receptor activity; G-protein signaling, coupled to |
| | | receptor 1 | | cyclic nucleotide second messenger; cell adhesion; cell-cell |
| | | | | signaling; chemotaxis; cytosolic calcium ion concentration |
| | | | | elevation; immune response; inflammatory response; integral to |
| | | | | plasma membrane; invasive growth; rhodopsin-like receptor |
| | | | | activity |
| 206214_at | 7941 | phospholipase A2, group | biological_process | 2-acetyl-1-alkylglycerophosphocholine esterase activity; 2- |
| | | VII (platelet-activating | | acetyl-1-alkylglycerophosphocholine esterase complex; |
| | | factor acetylhydrolase, | | extracellular; hydrolase activity; inflammatory response; lipid |
| | | plasma) | | catabolism; phospholipid binding |
| 209906_at | 719 | complement component 3a | biological_process | C3a anaphylatoxin receptor activity; G-protein coupled receptor |
| | | receptor 1 | | protein signaling pathway; cell motility; cellular defense |
| | | | | response; chemotaxis; circulation; complement component C3a |
| | | | | receptor activity; cytosolic calcium ion concentration elevation; |
| | | | | inflammatory response; integral to plasma membrane; |
| | | | | phosphatidylinositol-4,5-bisphosphate hydrolysis; rhodopsin-like |
| | | | | receptor activity; smooth muscle contraction |
| 211530_x_at | 3135 | HLA-G histocompatibility | biological_process | MHC class I receptor activity; antigen presentation, endogenous |
| | | antigen, class I, G | | antigen; antigen processing, endogenous antigen via MHC |
| | | | | class I; cellular defense response; integral to membrane; |
| | | | | perception of pest/pathogen/parasite |
| 211799_x_at | 3107 | major histocompatibility | biological_process | MHC class II receptor activity; class I major histocompatibility |
| | | complex, class I, C | | complex antigen; immune response; integral to membrane |
| 213975_s_at | 4069 | lysozyme (renal | biological_process | carbohydrate metabolism; cell wall catabolism; cytolysis; |
| | | amyloidosis) | | extracellular space; hydrolase activity, acting on glycosyl bonds; |
| | | | | inflammatory response; lysin activity; lysozyme activity |
| 217028_at | 7852 | chemokine (C—X—C | biological_process | C—C chemokine receptor activity; C—X—C chemokine receptor |
| | | motif) receptor 4 | | activity; G-protein coupled receptor protein signaling pathway; |
| | | | | activation of MAPK; apoptosis; chemotaxis; coreceptor activity; |
| | | | | cytoplasm; cytosolic calcium ion concentration elevation; |
| | | | | histogenesis and organogenesis; immune response; |
| | | | | inflammatory response; integral to plasma membrane; invasive |
| | | | | growth; neurogenesis; pathogenesis; response to viruses; |
| | | | | rhodopsin-like receptor activity |

Response to wounding

| | | | | |
|---|---|---|---|---|
| 203650_at | 10544 | protein C receptor, | biological_process | blood coagulation; inflammatory response; integral to plasma |
| | | endothelial (EPCR) | | membrane; receptor activity |
| 204122_at | 7305 | TYRO protein tyrosine | biological_process | cellular defense response; integral to plasma membrane; |
| | | kinase binding protein | | intracellular signaling cascade; receptor signaling protein activity |
| 204446_s_at | 240 | arachidonate 5- | biological_process | arachidonate 5-lipoxygenase activity; electron transport; |
| | | lipoxygenase | | inflammatory response; iron ion binding; leukotriene biosynthesis; |
| | | | | lipoxygenase activity; oxidoreductase activity |
| 205098_at | 1230 | chemokine (C—C motif) | biological_process | C—C chemokine receptor activity; G-protein signaling, coupled to |
| | | receptor 1 | | cyclic nucleotide second messenger; cell adhesion; cell-cell |
| | | | | signaling; chemotaxis; cytosolic calcium ion concentration elevation; |
| | | | | immune response; inflammatory response; integral to plasma |
| | | | | membrane; invasive growth; rhodopsin-like receptor activity |
| 206214_at | 7941 | phospholipase A2, | biological_process | 2-acetyl-1-alkylglycerophosphocholine esterase activity; 2-acetyl-1- |
| | | group VII (platelet- | | alkylglycerophosphocholine esterase complex; extracellular; |
| | | activating factor | | hydrolase activity; inflammatory response; lipid catabolism; |
| | | acetylhydrolase, | | phospholipid binding |
| | | plasma) | | |
| 209906_at | 719 | complement | biological_process | C3a anaphylatoxin receptor activity; G-protein coupled receptor |
| | | component 3a receptor 1 | | protein signaling pathway; cell motility; cellular defense response; |
| | | | | chemotaxis; circulation; complement component C3a receptor |
| | | | | activity; cytosolic calcium ion concentration elevation; inflammatory |
| | | | | response; integral to plasma membrane; phosphatidylinositol-4,5- |
| | | | | bisphosphate hydrolysis; rhodopsin-like receptor activity; smooth |
| | | | | muscle contraction |
| 211530_x_at | 3135 | HLA-G | biological_process | MHC class I receptor activity; antigen presentation, endogenous |
| | | histocompatibility | | antigen; antigen processing, endogenous antigen via MHC class I; |
| | | antigen, class I, G | | cellular defense response; integral to membrane; perception of |
| | | | | pest/pathogen/parasite |
| 213975_s_at | 4069 | lysozyme (renal | biological_process | carbohydrate metabolism; cell wall catabolism; cytolysis; |
| | | amyloidosis) | | extracellular space; hydrolase activity, acting on glycosyl bonds; |
| | | | | inflammatory response; lysin activity; lysozyme activity |
| 217028_at | 7852 | chemokine (C—X—C | biological_process | C—C chemokine receptor activity; C—X—C chemokine receptor |
| | | motif) receptor 4 | | activity; G-protein coupled receptor protein signaling pathway; |
| | | | | activation of MAPK; apoptosis; chemotaxis; coreceptor activity; |
| | | | | cytoplasm; cytosolic calcium ion concentration elevation; |
| | | | | histogenesis and organogenesis; immune response; inflammatory |
| | | | | response; integral to plasma membrane; invasive growth; |
| | | | | neurogenesis; pathogenesis; response to viruses; rhodopsin-like |
| | | | | receptor activity |

Response to stress

| | | | | |
|---|---|---|---|---|
| 201739_at | 6446 | serum/glucocorticoid | biological_process | ATP binding; apoptosis; cAMP-dependent protein kinase |
| | | regulated kinase | | activity; protein amino acid phosphorylation; protein kinase CK2 |
| | | | | activity; protein serine/threonine kinase activity; response to |
| | | | | stress; sodium ion transport; transferase activity |
| 201850_at | 822 | capping protein (actin | biological_process | F-actin capping protein complex; actin binding; barbed-end |
| | | filament), gelsolin-like | | actin capping activity; nucleus; protein complex assembly; |
| | | | | response to pest/pathogen/parasite |
| 201952_at | 214 | activated leukocyte cell | biological_process | antimicrobial humoral response (sensu Invertebrata); cell |
| | | adhesion molecule | | adhesion; cell adhesion molecule activity; integral to plasma |
| | | | | membrane; membrane fraction; receptor binding; signal |
| | | | | transduction |
| 202803_s_at | 3689 | integrin, beta 2 (antigen | biological_process | antimicrobial humoral response (sensu Invertebrata); cell |
| | | CD18 (p95), lymphocyte | | adhesion; cell adhesion receptor activity; integrin complex; |
| | | function-associated antigen | | integrin-mediated signaling pathway |
| | | 1; macrophage antigen 1 | | |
| | | (mac-1) beta subunit) | | |
| 203104_at | 1436 | colony stimulating factor 1 | biological_process | ATP binding; antimicrobial humoral response (sensu |
| | | receptor, formerly | | Invertebrata); cell proliferation; development; integral to plasma |
| | | McDonough feline sarcoma | | membrane; macrophage colony stimulating factor receptor |
| | | viral (v-fms) oncogene | | activity; protein amino acid phosphorylation; receptor activity; |
| | | homolog | | signal transduction; transferase activity; transmembrane |
| | | | | receptor protein tyrosine kinase signaling pathway |
| 203650_at | 10544 | protein C receptor, | biological_process | blood coagulation; inflammatory response; integral to plasma |
| | | endothelial (EPCR) | | membrane; receptor activity |
| 204122_at | 7305 | TYRO protein tyrosine | biological_process | cellular defense response; integral to plasma membrane; |
| | | kinase binding | | intracellular signaling cascade; receptor signaling protein |
| | | protein | | activity |
| 204446_s_at | 240 | arachidonate | biological_process | arachidonate 5-lipoxygenase activity; electron transport; |
| | | 5-lipoxygenase | | inflammatory response; iron ion binding; leukotriene |
| | | | | biosynthesis; lipoxygenase activity; oxidoreductase activity |
| 205098_at | 1230 | chemokine (C—C motif) | biological_process | C—C chemokine receptor activity; G-protein signaling, coupled |
| | | receptor 1 | | to cyclic nucleotide second messenger; cell adhesion; cell-cell |
| | | | | signaling; chemotaxis; cytosolic calcium ion concentration |
| | | | | elevation; immune response; inflammatory response; integral to |
| | | | | plasma membrane; invasive growth; rhodopsin-like receptor |
| | | | | activity |
| 206214_at | 7941 | phospholipase A2, group | biological_process | 2-acetyl-1-alkylglycerophosphocholine esterase activity; 2- |
| | | VII (platelet-activating | | acetyl-1-alkylglycerophosphocholine esterase complex; |
| | | factor acetylhydrolase, | | extracellular; hydrolase activity; inflammatory response; lipid |
| | | plasma) | | catabolism; phospholipid binding |
| 209906_at | 719 | complement component 3a | biological_process | C3a anaphylatoxin receptor activity; G-protein coupled receptor |
| | | receptor 1 | | protein signaling pathway; cell motility; cellular defense |
| | | | | response; chemotaxis; circulation; complement component C3a |
| | | | | receptor activity; cytosolic calcium ion concentration elevation; |
| | | | | inflammatory response; integral to plasma membrane; |
| | | | | phosphatidylinositol-4,5-bisphosphate hydrolysis; rhodopsin- |
| | | | | like receptor activity; smooth muscle contraction |
| 211530_x_at | 3135 | HLA-G histocompatibility | biological_process | MHC class I receptor activity; antigen presentation, |
| | | antigen, class I, G | | endogenous antigen; antigen processing, endogenous antigen |
| | | | | via MHC class I; cellular defense response; integral to |
| | | | | membrane; perception of pest/pathogen/parasite |
| 213975_s_at | 4069 | lysozyme (renal | biological_process | carbohydrate metabolism; cell wall catabolism; cytolysis; |
| | | amyloidosis) | | extracellular space; hydrolase activity, acting on glycosyl |
| | | | | bonds; inflammatory response; lysin activity; lysozyme activity |
| 217028_at | 7852 | chemokine (C—X—C | biological_process | C—C chemokine receptor activity; C—X—C chemokine receptor |
| | | motif) receptor 4 | | activity; G-protein coupled receptor protein signaling pathway; |
| | | | | activation of MAPK; apoptosis; chemotaxis; coreceptor activity; |
| | | | | cytoplasm; cytosolic calcium ion concentration elevation; |
| | | | | histogenesis and organogenesis; immune response; |
| | | | | inflammatory response; integral to plasma membrane; invasive |
| | | | | growth; neurogenesis; pathogenesis; response to viruses; |
| | | | | rhodopsin-like receptor activity |

Response to stress

| | | | | |
|---|---|---|---|---|
| 201739_at | 6446 | serum/glucocorticoid | biological_process | ATP binding; apoptosis; cAMP-dependent protein kinase |
| | | regulated kinase | | activity; protein amino acid phosphorylation; protein kinase |
| | | | | CK2 activity; protein serine/threonine kinase activity; response |
| | | | | to stress; sodium ion transport; transferase activity |
| 201850_at | 822 | capping protein (actin | biological_process | F-actin capping protein complex; actin binding; barbed-end |
| | | filament), gelsolin-like | | actin capping activity; nucleus; protein complex assembly; |
| | | | | response to pest/pathogen/parasite |
| 201952_at | 214 | activated leukocyte cell | biological_process | antimicrobial humoral response (sensu Invertebrata); cell |
| | | adhesion molecule | | adhesion; cell adhesion molecule activity; integral to plasma |
| | | | | membrane; membrane fraction; receptor binding; signal |
| | | | | transduction |
| 202803_s_at | 3689 | integrin, beta 2 (antigen | biological_process | antimicrobial humoral response (sensu Invertebrata); cell |
| | | CD18 (p95), lymphocyte | | adhesion; cell adhesion receptor activity; integrin complex; |
| | | function-associated antigen | | integrin-mediated signaling pathway |
| | | 1; macrophage antigen 1 | | |
| | | (mac-1) beta subunit) | | |
| 203104_at | 1436 | colony stimulating factor 1 | biological_process | ATP binding; antimicrobial humoral response (sensu |
| | | receptor, formerly | | Invertebrata); cell proliferation; development; integral to |
| | | McDonough feline sarcoma | | plasma membrane; macrophage colony stimulating factor |
| | | viral (v-fms) oncogene | | receptor activity; protein amino acid phosphorylation; receptor |
| | | homolog | | activity; signal transduction; transferase activity; |
| | | | | transmembrane receptor protein tyrosine kinase signaling |
| | | | | pathway |
| 203650_at | 10544 | protein C receptor, | biological_process | blood coagulation; inflammatory response; integral to plasma |
| | | endothelial (EPCR) | | membrane; receptor activity |
| 204122_at | 7305 | TYRO protein tyrosine | biological_process | cellular defense response; integral to plasma membrane; |
| | | kinase binding | | intracellular signaling cascade; receptor signaling protein |
| | | protein | | activity |
| 204446_s_at | 240 | arachidonate | biological_process | arachidonate 5-lipoxygenase activity; electron transport; |
| | | 5-lipoxygenase | | inflammatory response; iron ion binding; leukotriene |
| | | | | biosynthesis; lipoxygenase activity; oxidoreductase activity |
| 205098_at | 1230 | chemokine (C—C motif) | biological_process | C—C chemokine receptor activity; G-protein signaling, coupled |
| | | receptor 1 | | to cyclic nucleotide second messenger; cell adhesion; cell-cell |
| | | | | signaling; chemotaxis; cytosolic calcium ion concentration |
| | | | | elevation; immune response; inflammatory response; integral |
| | | | | to plasma membrane; invasive growth; rhodopsin-like |
| | | | | receptor activity |
| 206214_at | 7941 | phospholipase A2, group | biological_process | 2-acetyl-1-alkylglycerophosphocholine esterase activity; 2- |
| | | VII (platelet-activating | | acetyl-1-alkylglycerophosphocholine esterase complex; |
| | | factor acetylhydrolase, | | extracellular; hydrolase activity; inflammatory response; lipid |
| | | plasma) | | catabolism; phospholipid binding |
| 209906_at | 719 | complement component 3a | biological_process | C3a anaphylatoxin receptor activity; G-protein coupled |
| | | receptor 1 | | receptor protein signaling pathway; cell motility; cellular |
| | | | | defense response; chemotaxis; circulation; complement |
| | | | | component C3a receptor activity; cytosolic calcium ion |
| | | | | concentration elevation; inflammatory response; integral to |
| | | | | plasma membrane; phosphatidylinositol-4,5-bisphosphate |
| | | | | hydrolysis; rhodopsin-like receptor activity; smooth muscle |
| | | | | contraction |
| 211530_x_at | 3135 | HLA-G histocompatibility | biological_process | MHC class I receptor activity; antigen presentation, |
| | | antigen, class I, G | | endogenous antigen; antigen processing, endogenous |
| | | | | antigen via MHC class I; cellular defense response; integral to |
| | | | | membrane; perception of pest/pathogen/parasite |
| 213975_s_at | 4069 | lysozyme (renal | biological_process | carbohydrate metabolism; cell wall catabolism; cytolysis; |
| | | amyloidosis) | | extracellular space; hydrolase activity, acting on glycosyl |
| | | | | bonds; inflammatory response; lysin activity; lysozyme activity |
| 217028_at | 7852 | chemokine (C—X—C | biological_process | C—C chemokine receptor activity; C—X—C chemokine receptor |
| | | motif) receptor 4 | | activity; G-protein coupled receptor protein signaling pathway; |
| | | | | activation of MAPK; apoptosis; chemotaxis; coreceptor |
| | | | | activity; cytoplasm; cytosolic calcium ion concentration |
| | | | | elevation; histogenesis and organogenesis; immune |
| | | | | response; inflammatory response; integral to plasma |
| | | | | membrane; invasive growth; neurogenesis; pathogenesis; |
| | | | | response to viruses; rhodopsin-like receptor activity |

Inflammatory response

| | | | | |
|---|---|---|---|---|
| 203650_at | 10544 | protein C receptor, | biological_process | blood coagulation; inflammatory response; integral to plasma |
| | | endothelial (EPCR) | | membrane; receptor activity |
| 204446_s_at | 240 | arachidonate 5- | biological_process | arachidonate 5-lipoxygenase activity; electron transport; inflammatory |
| | | lipoxygenase | | response; iron ion binding; leukotriene biosynthesis; lipoxygenase |
| | | | | activity; oxidoreductase activity |
| 205098_at | 1230 | chemokine (C—C | biological_process | C—C chemokine receptor activity; G-protein signaling, coupled to |
| | | motif) receptor 1 | | cyclic nucleotide second messenger; cell adhesion; cell-cell signaling; |
| | | | | chemotaxis; cytosolic calcium ion concentration elevation; immune |
| | | | | response; inflammatory response; integral to plasma membrane; |
| | | | | invasive growth; rhodopsin-like receptor activity |
| 206214_at | 7941 | phospholipase A2, | biological_process | 2-acetyl-1-alkylglycerophosphocholine esterase activity; 2-acetyl-1- |
| | | group VII (platelet- | | alkylglycerophosphocholine esterase complex; extracellular; |
| | | activating factor | | hydrolase activity; inflammatory response; lipid catabolism; |
| | | acetylhydrolase, | | phospholipid binding |
| | | plasma) | | |
| 209906_at | 719 | complement | biological_process | C3a anaphylatoxin receptor activity; G-protein coupled receptor |
| | | component 3a | | protein signaling pathway; cell motility; cellular defense response; |
| | | receptor 1 | | chemotaxis; circulation; complement component C3a receptor |
| | | | | activity; cytosolic calcium ion concentration elevation; inflammatory |
| | | | | response; integral to plasma membrane; phosphatidylinositol-4,5- |
| | | | | bisphosphate hydrolysis; rhodopsin-like receptor activity; smooth |
| | | | | muscle contraction |
| 213975_s_at | 4069 | lysozyme (renal | biological_process | carbohydrate metabolism; cell wall catabolism; cytolysis; extracellular |
| | | amyloidosis) | | space; hydrolase activity, acting on glycosyl bonds; inflammatory |
| | | | | response; lysin activity; lysozyme activity |
| 217028_at | 7852 | chemokine (C—X—C | biological_process | C—C chemokine receptor activity; C—X—C chemokine |
| | | motif) receptor 4 | | receptor activity; G-protein coupled receptor protein signaling pathway; |
| | | | | activation of MAPK; apoptosis; chemotaxis; |
| | | | | coreceptor activity; cytoplasm; |
| | | | | cytosolic calcium ion concentration elevation; histogenesis and |
| | | | | organogenesis; immune response; inflammatory response; integral to |
| | | | | plasma membrane; invasive growth; neurogenesis; pathogenesis; |
| | | | | response to viruses; rhodopsin-like receptor activity |

Innate immune response

| | | | | |
|---|---|---|---|---|
| 203650_at | 10544 | protein C receptor, | biological_process | blood coagulation; inflammatory response; integral to plasma |
| | | endothelial (EPCR) | | membrane; receptor activity |
| 204446_s_at | 240 | arachidonate 5- | biological_process | arachidonate 5-lipoxygenase activity; electron transport; inflammatory |
| | | lipoxygenase | | response; iron ion binding; leukotriene biosynthesis; lipoxygenase |
| | | | | activity; oxidoreductase activity |
| 205098_at | 1230 | chemokine (C—C | biological_process | C—C chemokine receptor activity; G-protein signaling, coupled to |
| | | motif) receptor 1 | | cyclic nucleotide second messenger; cell adhesion; cell-cell signaling; |
| | | | | chemotaxis; cytosolic calcium ion concentration elevation; immune |
| | | | | response; inflammatory response; integral to plasma membrane; |
| | | | | invasive growth; rhodopsin-like receptor activity |
| 206214_at | 7941 | phospholipase A2, | biological_process | 2-acetyl-1-alkylglycerophosphocholine esterase activity; 2-acetyl-1- |
| | | group VII (platelet- | | alkylglycerophosphocholine esterase complex; extracellular; |
| | | activating factor | | hydrolase activity; inflammatory response; lipid catabolism; |
| | | acetylhydrolase, | | phospholipid binding |
| | | plasma) | | |
| 209906_at | 719 | complement | biological_process | C3a anaphylatoxin receptor activity; G-protein coupled receptor |
| | | component 3a | | protein signaling pathway; cell motility; cellular defense response; |
| | | receptor 1 | | chemotaxis; circulation; complement component C3a receptor |
| | | | | activity; cytosolic calcium ion concentration elevation; inflammatory |
| | | | | response; integral to plasma membrane; phosphatidylinositol-4,5- |
| | | | | bisphosphate hydrolysis; rhodopsin-like receptor activity; smooth |
| | | | | muscle contraction |
| 213975_s_at | 4069 | lysozyme (renal | biological_process | carbohydrate metabolism; cell wall catabolism; cytolysis; extracellular |
| | | amyloidosis) | | space; hydrolase activity, acting on glycosyl bonds; inflammatory |
| | | | | response; lysin activity; lysozyme activity |
| 217028_at | 7852 | chemokine (C—X—C | biological_process | C—C chemokine receptor activity; C—X—C chemokine receptor |
| | | motif) receptor 4 | | activity; G-protein coupled receptor protein signaling pathway; |
| | | | | activation of MAPK; apoptosis; chemotaxis; coreceptor |
| | | | | activity; cytoplasm; |
| | | | | cytosolic calcium ion concentration elevation; histogenesis and |
| | | | | organogenesis; immune response; inflammatory response; integral to |
| | | | | plasma membrane; invasive growth; neurogenesis; pathogenesis; |
| | | | | response to viruses; rhodopsin-like receptor activity |

Response to external stimulus

| | | | | |
|---|---|---|---|---|
| 201422_at | 10437 | interferon, gamma- | biological_process | extracellular; immune response; lysosome; oxidoreductase |
| | | inducible protein 30 | | activity |
| 201850_at | 822 | capping protein (actin | biological_process | F-actin capping protein complex; actin binding; barbed-end |
| | | filament), gelsolin-like | | actin capping activity; nucleus; protein complex assembly; |
| | | | | response to pest/pathogen/parasite |
| 201952_at | 214 | activated leukocyte cell | biological_process | antimicrobial humoral response (sensu Invertebrata); cell |
| | | adhesion molecule | | adhesion; cell adhesion molecule activity; integral to plasma |
| | | | | membrane; membrane fraction; receptor binding; signal |
| | | | | transduction |
| 202803_s_at | 3689 | integrin, beta 2 (antigen | biological_process | antimicrobial humoral response (sensu Invertebrata); cell |
| | | CD18 (p95), lymphocyte | | adhesion; cell adhesion receptor activity; integrin complex; |
| | | function-associated antigen | | integrin-mediated signaling pathway |
| | | 1; macrophage antigen 1 | | |
| | | (mac-1) (beta subunit) | | |
| 202901_x_at | 1520 | cathepsin S | biological_process | cathepsin S activity; hydrolase activity; immune response; |
| | | | | lysosome; proteolysis and peptidolysis |
| 203104_at | 1436 | colony stimulating factor 1 | biological_process | ATP binding; antimicrobial humoral response (sensu |
| | | receptor, formerly | | Invertebrata); cell proliferation; development; integral to plasma |
| | | McDonough feline sarcoma | | membrane; macrophage colony stimulating factor receptor |
| | | viral (v-fms) oncogene | | activity; protein amino acid phosphorylation; receptor activity; |
| | | homolog | | signal transduction; transferase activity; transmembrane |
| | | | | receptor protein tyrosine kinase signaling pathway |
| 203382_s_at | 348 | apolipoprotein E | biological_process | cholesterol metabolism; circulation; development; heparin |
| | | | | binding; immune response; lipid binding; lipid metabolism; lipid |
| | | | | transport; lipid transporter activity; receptor binding |
| 203650_at | 10544 | protein C receptor, | biological_process | blood coagulation; inflammatory response; integral to plasma |
| | | endothelial (EPCR) | | membrane; receptor activity |
| 204122_at | 7305 | TYRO protein tyrosine | biological_process | cellular defense response; integral to plasma membrane; |
| | | kinase binding protein | | intracellular signaling cascade; receptor signaling protein |
| | | | | activity |
| 204446_s_at | 240 | arachidonate 5- | biological_process | arachidonate 5-lipoxygenase activity; electron transport; |
| | | lipoxygenase | | inflammatory response; iron ion binding; leukotriene |
| | | | | biosynthesis; lipoxygenase activity; oxidoreductase activity |
| 205098_at | 1230 | chemokine (C—C motif) | biological_process | C—C chemokine receptor activity; G-protein signaling, coupled to |
| | | receptor 1 | | cyclic nucleotide second messenger; cell adhesion; cell-cell |
| | | | | signaling; chemotaxis; cytosolic calcium ion concentration |
| | | | | elevation; immune response; inflammatory response; integral to |
| | | | | plasma membrane; invasive growth; rhodopsin-like receptor |
| | | | | activity |
| 206214_at | 7941 | phospholipase A2, group | biological_process | 2-acetyl-1-alkylglycerophosphocholine esterase activity; 2- |
| | | VII (platelet-activating | | acetyl-1-alkylglycerophosphocholine esterase complex; |
| | | factor acetylhydrolase, | | extracellular; hydrolase activity; inflammatory response; lipid |
| | | plasma) | | catabolism; phospholipid binding |
| 209906_at | 719 | complement component 3a | biological_process | C3a anaphylatoxin receptor activity; G-protein coupled receptor |
| | | receptor 1 | | protein signaling pathway; cell motility; cellular defense |
| | | | | response; chemotaxis; circulation; complement component C3a |
| | | | | receptor activity; cytosolic calcium ion concentration elevation; |
| | | | | inflammatory response; integral to plasma membrane; |
| | | | | phosphatidylinositol-4,5-bisphosphate hydrolysis; rhodopsin-like |
| | | | | receptor activity; smooth muscle contraction |
| 211530_x_at | 3135 | HLA-G histocompatibility | biological_process | MHC class I receptor activity; antigen presentation, endogenous |
| | | antigen, class I, G | | antigen; antigen processing, endogenous antigen via MHC |
| | | | | class I; cellular defense response; integral to membrane; |
| | | | | perception of pest/pathogen/parasite |
| 211799_x_at | 3107 | major histocompatibility | biological_process | MHC class II receptor activity; class I major histocompatibility |
| | | complex, class I, C | | complex antigen; immune response; integral to membrane |
| 213975_s_at | 4069 | lysozyme (renal | biological_process | carbohydrate metabolism; cell wall catabolism; cytolysis; |
| | | amyloidosis) | | extracellular space; hydrolase activity, acting on glycosyl bonds; |
| | | | | inflammatory response; lysin activity; lysozyme activity |
| 217028_at | 7852 | chemokine (C—X—C | biological_process | C—C chemokine receptor activity; C—X—C chemokine receptor |
| | | motif) receptor 4 | | activity; G-protein coupled receptor protein signaling pathway; |
| | | | | activation of MAPK; apoptosis; chemotaxis; coreceptor activity; |
| | | | | cytoplasm; cytosolic calcium ion concentration elevation; |
| | | | | histogenesis and organogenesis; immune response; |
| | | | | inflammatory response; integral to plasma membrane; invasive |
| | | | | growth; neurogenesis; pathogenesis; response to viruses; |
| | | | | rhodopsin-like receptor activity |

Catabolism

| | | | | |
|---|---|---|---|---|
| 202295_s_at | 1512 | cathepsin H | biological_process | cathepsin H activity; hydrolase activity; lysosome; proteolysis |
| | | | | and peptidolysis |
| 202901_x_at | 1520 | cathepsin S | biological_process | cathepsin S activity; hydrolase activity; immune response; |
| | | | | lysosome; proteolysis and peptidolysis |
| 203649_s_at | 5320 | phospholipase A2, group | biological_process | calcium ion binding; calcium-dependent cytosolic |
| | | IIA (platelets, synovial | | phospholipase A2 activity; calcium-dependent secreted |
| | | fluid) | | phospholipase A2 activity; calcium-independent cytosolic |
| | | | | phospholipase A2 activity; hydrolase activity; lipid catabolism; |
| | | | | membrane |
| 203936_s_at | 4318 | matrix metalloproteinase 9 | biological_process | collagen catabolism; collagenase activity; extracellular matrix; |
| | | (gelatinase B, 92 kDa | | extracellular space; gelatinase B activity; hydrolase activity; |
| | | gelatinase, 92 kDa type IV | | zinc ion binding |
| | | collagenase) | | |
| 206214_at | 7941 | phospholipase A2, group | biological_process | 2-acetyl-1-alkylglycerophosphocholine esterase activity; 2- |
| | | VII (platelet-activating | | acetyl-1-alkylglycerophosphocholine esterase complex; |
| | | factor acetylhydrolase, | | extracellular; hydrolase activity; inflammatory response; lipid |
| | | plasma) | | catabolism; phospholipid binding |
| 207332_s_at | 7037 | transferrin receptor (p90, | biological_process | endocytosis; endosome; extracellular; integral to plasma |
| | | CD71) | | membrane; iron ion homeostasis; iron ion transport; peptidase |
| | | | | activity; proteolysis and peptidolysis; receptor activity; |
| | | | | transferrin receptor activity |
| 213274_s_at | 1508 | cathepsin B | biological_process | cathepsin B activity; hydrolase activity; intracellular; lysosome; |
| | | | | proteolysis and peptidolysis |
| 213510_x_at | 220594 | TL132 protein | biological_process | cysteine-type endopeptidase activity; ubiquitin C-terminal |
| | | | | hydrolase activity; ubiquitin-dependent protein catabolism |
| 213975_s_at | 4069 | lysozyme (renal | biological_process | carbohydrate metabolism; cell wall catabolism; cytolysis; |
| | | amyloidosis) | | extracellular space; hydrolase activity, acting on glycosyl |
| | | | | bonds; inflammatory response; lysin activity; lysozyme activity |
| 214012_at | 51752 | type 1 tumor necrosis factor | biological_process | aminopeptidase activity; membrane alanyl aminopeptidase |
| | | receptor shedding | | activity; metallopeptidase activity; proteolysis and peptidolysis; |
| | | aminopeptidase regulator | | zinc ion binding |
| 217983_s_at | 8635 | ribonuclease 6 precursor | biological_process | RNA catabolism; extracellular; ribonuclease activity |
| 35820_at | 2760 | GM2 ganglioside activator | biological_process | glycolipid catabolism; glycosphingolipid metabolism; lysosome; |
| | | protein | | sphingolipid activator protein activity; sphingolipid catabolism |

Colony morphology

| | | | | |
|---|---|---|---|---|
| 203186_s_at | 6275 | S100 calcium binding | biological_process | calcium ion binding; invasive growth |
| | | protein A4 (calcium | | |
| | | protein, calvasculin, | | |
| | | metastasin, murine | | |
| | | placental homolog) | | |
| 205098_at | 1230 | chemokine (C—C motif) | biological_process | C—C chemokine receptor activity; G-protein signaling, coupled to |
| | | receptor 1 | | cyclic nucleotide second messenger; cell adhesion; cell-cell |
| | | | | signaling; chemotaxis; cytosolic calcium ion concentration elevation; |
| | | | | immune response; inflammatory response; integral to plasma |
| | | | | membrane; invasive growth; rhodopsin-like receptor activity |
| 217028_at | 7852 | chemokine (C—X—C | biological_process | C—C chemokine receptor activity; C—X—C chemokine receptor |
| | | motif) receptor 4 | | activity; G-protein coupled receptor protein signaling pathway; |
| | | | | activation of MAPK; apoptosis; chemotaxis; coreceptor activity; |
| | | | | cytoplasm; cytosolic calcium ion concentration elevation; |
| | | | | histogenesis and organogenesis; immune response; inflammatory |
| | | | | response; integral to plasma membrane; invasive growth; |
| | | | | neurogenesis; pathogenesis; response to viruses; rhodopsin-like |
| | | | | receptor activity |

Invasive growth

| | | | | |
|---|---|---|---|---|
| 203186_s_at | 6275 | S100 calcium binding | biological_process | calcium ion binding; invasive growth |
| | | protein A4 (calcium | | |
| | | protein, calvasculin, | | |
| | | metastasin, murine | | |
| | | placental homolog) | | |
| 205098_at | 1230 | chemokine (C—C motif) | biological_process | C—C chemokine receptor activity; G-protein signaling, coupled to |
| | | receptor 1 | | cyclic nucleotide second messenger; cell adhesion; cell-cell |
| | | | | signaling; chemotaxis; cytosolic calcium ion concentration |
| | | | | elevation; immune response; inflammatory response; integral to |
| | | | | plasma membrane; invasive growth; rhodopsin-like receptor activity |
| 217028_at | 7852 | chemokine(C—X—C | biological_process | C—C chemokine receptor activity; C—X—C chemokine receptor |
| | | motif) receptor 4 | | activity; G-protein coupled receptor protein signaling pathway; |
| | | | | activation of MAPK; apoptosis; chemotaxis; coreceptor activity; |
| | | | | cytoplasm; cytosolic calcium ion concentration elevation; |
| | | | | histogenesis and organogenesis; immune response; inflammatory |
| | | | | response; integral to plasma membrane; invasive growth; |
| | | | | neurogenesis; pathogenesis; response to viruses; rhodopsin-like |
| | | | | receptor activity |

Cystolic calcium ion concentration elevation

| | | | | |
|---|---|---|---|---|
| 205098_at | 1230 | chemokine (C—C | biological_process | C—C chemokine receptor activity; G-protein signaling, coupled |
| | | motif) receptor 1 | | to cyclic nucleotide second messenger; cell adhesion; cell-cell |
| | | | | signaling; chemotaxis; |
| | | | | cytosolic calcium ion concentration elevation; immune response; |
| | | | | inflammatory response; integral to plasma membrane; invasive growth; |
| | | | | rhodopsin-like receptor activity |
| 209906_at | 719 | complement | biological_process | C3a anaphylatoxin receptor activity; G-protein coupled receptor protein |
| | | component 3a | | signaling pathway; cell motility; cellular defense response; chemotaxis; |
| | | receptor 1 | | circulation; complement component C3a receptor activity; cytosolic |
| | | | | calcium ion concentration elevation; inflammatory response; |
| | | | | integral to plasma membrane; |
| | | | | phosphatidylinositol-4,5-bisphosphate hydrolysis; |
| | | | | rhodopsin-like receptor activity; smooth muscle contraction |
| 217028_at | 7852 | chemokine | biological_process | C—C chemokine receptor activity; C—X—C chemokine receptor |
| | | (C—X—C motif) | | activity; G-protein coupled receptor protein signaling pathway; |
| | | receptor 4 | | activation of MAPK; apoptosis; chemotaxis; coreceptor |
| | | | | activity; cytoplasm; |
| | | | | cytosolic calcium ion concentration elevation; histogenesis |
| | | | | and organogenesis; immune response; |
| | | | | inflammatory response; integral to plasma membrane; invasive growth; |
| | | | | neurogenesis; pathogenesis; response to viruses; rhodopsin-like receptor |
| | | | | activity |

Cellular morphogenesis

| | | | | |
|---|---|---|---|---|
| 203186_s_at | 6275 | S100 calcium binding | biological_process | calcium ion binding; invasive growth |
| | | protein A4 (calcium | | |
| | | protein, calvasculin, | | |
| | | metastasin, murine | | |
| | | placental homolog) | | |
| 205098_at | 1230 | chemokine (C—C motif) | biological_process | C—C chemokine receptor activity; G-protein signaling, coupled to |
| | | receptor 1 | | cyclic nucleotide second messenger; cell adhesion; cell-cell |
| | | | | signaling; chemotaxis; cytosolic calcium ion concentration |
| | | | | elevation; immune response; inflammatory response; integral to |
| | | | | plasma membrane; invasive growth; rhodopsin-like receptor activity |
| 217028_at | 7852 | chemokine (C—X—C | biological_process | C—C chemokine receptor activity; C—X—C chemokine receptor |
| | | motif) receptor 4 | | activity; G-protein coupled receptor protein signaling pathway; |
| | | | | activation of MAPK; apoptosis; chemotaxis; coreceptor activity; |
| | | | | cytoplasm; cytosolic calcium ion concentration elevation; |
| | | | | histogenesis and organogenesis; immune response; inflammatory |
| | | | | response; integral to plasma membrane; invasive growth; |
| | | | | neurogenesis; pathogenesis; response to viruses; rhodopsin-like |
| | | | | receptor activity |

Cell adhesion

| | | | | |
|---|---|---|---|---|
| 201952_at | 214 | activated leukocyte cell | biological_process | antimicrobial humoral response (sensu Invertebrata); cell |
| | | adhesion molecule | | adhesion; cell adhesion molecule activity; integral to plasma |
| | | | | membrane; membrane fraction; receptor binding; signal |
| | | | | transduction |
| 202803_s_at | 3689 | integrin, beta 2 (antigen | biological_process | antimicrobial humoral response (sensu Invertebrata); cell |
| | | CD18 (p95), lymphocyte | | adhesion; cell adhesion receptor activity; integrin complex; |
| | | function-associated | | integrin-mediated signaling pathway |
| | | antigen 1; macrophage | | |
| | | antigen 1 (mac-1) beta | | |
| | | subunit) | | |
| 204438_at | 4360 | mannose receptor, C type 1 | biological_process | calcium ion binding; heterophilic cell adhesion; integral to |
| | | | | plasma membrane; mannose binding; pinocytosis; receptor |
| | | | | activity; receptor mediated endocytosis; sugar binding |
| 204620_s_at | 1462 | chondroitin sulfate | biological_process | calcium ion binding; cell recognition; development; extracellular |
| | | proteoglycan 2 (versican) | | matrix; heterophilic cell adhesion; hyaluronic acid binding; sugar |
| | | | | binding |
| 205098_at | 1230 | chemokine (C—C motif) | biological_process | C—C chemokine receptor activity; G-protein signaling; coupled to |
| | | receptor 1 | | cyclic nucleotide second messenger; cell adhesion; cell-cell |
| | | | | signaling; chemotaxis; cytosolic calcium ion concentration |
| | | | | elevation; immune response; inflammatory response; integral to |
| | | | | plasma membrane; invasive growth; rhodopsin-like receptor |
| | | | | activity |
| 205786_s_at | 3684 | integrin, alpha M | biological_process | cell adhesion; cell adhesion receptor activity; integrin complex |
| | | (complement component | | |
| | | receptor 3, alpha; also | | |
| | | known as CD11b (p170), | | |
| | | macrophage antigen | | |
| | | alpha polypeptide) | | |
| 212014_x_at | 960 | CD44 antigen (homing | biological_process | cell adhesion receptor activity; cell-cell adhesion; cell-matrix |
| | | function and Indian blood | | adhesion; collagen binding; hyaluronic acid binding; integral to |
| | | group system) | | plasma membrane; receptor activity |
| 216442_x_at | 2335 | fibronectin 1 | biological_process | cell adhesion; cell adhesion molecule activity; cell motility; |
| | | | | extracellular matrix; extracellular space; signal transduction; |
| | | | | soluble fraction |

Macromolecule catabolism

| | | | | |
|---|---|---|---|---|
| 202295_s_at | 1512 | cathepsin H | biological_process | cathepsin H activity; hydrolase activity; lysosome; proteolysis |
| | | | | and peptidolysis |
| 202901_x_at | 1520 | cathepsin S | biological_process | cathepsin S activity; hydrolase activity; immune response; |
| | | | | lysosome; proteolysis and peptidolysis |
| 203936_s_at | 4318 | matrix metalloproteinase 9 | biological_process | collagen catabolism; collagenase activity; extracellular matrix; |
| | | (gelatinase B, 92 kDa | | extracellular space; gelatinase B activity; hydrolase activity; |
| | | gelatinase, 92 kDa type IV | | zinc ion binding |
| | | collagenase) | | |
| 207332_s_at | 7037 | transferrin receptor (p90, | biological_process | endocytosis; endosome; extracellular; integral to plasma |
| | | CD71) | | membrane; iron ion homeostasis; iron ion transport; peptidase |
| | | | | activity; proteolysis and peptidolysis; receptor activity, |
| | | | | transferrin receptor activity |
| 213274_s_at | 1508 | cathepsin B | biological_process | cathepsin B activity; hydrolase activity; intracellular; |
| | | | | lysosome; proteolysis and peptidolysis |
| 213510_x_at | 220594 | TL132 protein | biological_process | cysteine-type endopeptidase activity; ubiquitin C-terminal |
| | | | | hydrolase activity; ubiquitin-dependent protein catabolism |
| 214012_at | 51752 | type 1 tumor necrosis factor | biological_process | aminopeptidase activity; membrane alanyl aminopeptidase |
| | | receptor shedding | | activity; metallopeptidase activity; proteolysis and |
| | | aminopeptidase regulator | | peptidolysis; zinc ion binding |
| 217983_s_at | 8635 | ribonuclease 6 precursor | biological_process | RNA catabolism; extracellular; ribonuclease activity |

The purpose of this experiment was not to solve whether the usf1s2 SNP is directly causative to FCHL. More complex functional studies need to be performed before any conclusions of the functional significance of a single non-coding SNP can be drawn. However, these preliminary data combined with the across species conservation would imply that the DNA region flanking the susceptibility haplotype contains an element affecting transcriptional regulation. The data also suggest that the element is more likely to be a C is acting type regulator rather than a direction-independent enhancer element.

### EXAMPLE 5

### Experimental Setup

### Methods In Examples 1 to 4

The Finnish FCHL families were recruited in the Helsinki, Turku and Kuopio University Central Hospitals, as described earlier^{4,9}. Each subject provided a written informed consent prior to participating in the study. All samples were collected in accordance with the Helsinki declaration, and the ethics committees of the participating centers approved the study design. The inclusion criteria for the FCHL probands were as follows⁴: 1) serum TC and/or TGs>90^{th} age-sex specific Finnish population percentiles⁴, but if the proband had only one elevated lipid trait, a first-degree relative had to have the combined phenotype; 2) age>30 years and <55 for males and <65 years for females; 3) at least a 50% stenosis in one or more coronary arteries in coronary angiography. Exclusion criteria for the FCHL probands were type 1 DM, hepatic or renal disease, and hypothyroidism. Familial hypercholesterolemia was excluded from each pedigree by determining the LDL-receptor status of the proband by the lymphocyte culture method⁴. If the above mentioned criteria were fulfilled, families with at least two affected members were included in the study, and all the accessible family members were examined. Two traits were analyzed: FCHL and TGs. For the FCHL trait, family members were scored as affected according to the same diagnostic criteria as in our original linkage study⁴ using the Finnish age-sex specific 90^{th} percentiles for high TC and high TGs, available from the web site of the National Public Health Institute, Finland. These ascertainment criteria are fully comparable with the original criteria¹. For analysis of TGs, family members with TG levels≧90^{th} Finnish age-sex specific population percentile were coded as affected. In addition to the FCHL and TG traits, the combination of the usf1s1-usf1s2 SNPs, which resulted in the significant haplotypes for the FCHL and TG traits, was also analyzed using the apolipoprotein B (apoB), LDL peak particle size and TC traits. For apoB and TC, the 90^{th} age-sex specific Finnish population percentiles, publicly available from the web site of the National Public Health Institute, Finland, were used. For LDL peak particle size, the cut point of 25.5 nm was used to code individuals with small LDL particles as affected. Although LDL-C is an important component trait of FCHL, serum TC was used instead in the ascertainment of the Finnish FCHL families as well as in the statistical analyses of the SNPs forming the USF1 susceptibility haplotype. The reasoning for this is the significant hypertriglyceridemia associated with FCHL. The Friedewald formula is generally not recommended when TGs are over (400 mg/dl i.e. 4.4 mmol/l), which is often the case with hypertriglyceridemic FCHL family members. In addition, the population percentile points of LDL-C could not be estimated when including this factor, as we currently don't have population percentiles for LDL-C.

### Biochemical Analyses

Serum lipid parameters and LDL peak particle size were measured as described earlier^{4,9,39}. Probands or hyperlipidemic relatives who used lipid-lowering drugs were studied after their treatment was withheld for 4 weeks. In the 60 FCHL families, DNA and lipid measurements were available for 721 and 771 family members, respectively. In these 60 FCHL families, there were 226 individuals with TC>90% age-sex specific Finnish population percentile, 220 with TGs>90% age-sex specific percentile, 321 with TC and/or TGs>90% age-sex specific percentile; and 125 individuals with both TC and TGs>90% age-sex specific percentiles, respectively. A total of 96 men and 124 women exhibited high TGs (>age-sex 90^{th} percentile).

### Sequencing, Genotyping and Sequence Annotations

The TXNIP gene was sequenced in the 60 FCHL probands and the APOA2, RXRG, and USF1 genes in the 31 probands of the original linkage study⁴. For TXNIP and USF1, 2000 bp upstream from the 5′ end of the gene were also sequenced. For USF1, the DNA binding domain was also sequenced in the remaining 29 probands. For all genes, both exons and introns were sequenced, except for the large 44,261-bp RXRG gene where only exons and 100 bp exon-intron boundaries were sequenced. Sequencing was done in both directions to identify heterozygotes reliably. Sequencing was performed according to the Big Dye Terminator Cycle Sequencing protocol (Applied Biosystems), with minor modifications and the samples separated with the automated DNA sequencer ABI 377XL (Applied Biosystems). Sequence contigs were assembled through use of Sequencher software (GeneCodes). The dbSNP and CELERA databases were used to select SNPs. Pyrosequencing and solid-phase minisequencing techniques were applied for SNP genotyping, as described earlier^{4,40}. Pyrosequencing was performed using the PSQ96 instrument and the SNP Reagent kit (Pyrosequencing AB). Every SNP was first genotyped in a subset of 46 family members from 18 of the 60 FCHL families. If the SNP was polymorphic (minor allele frequency>10% in this subset), the SNP was genotyped in 238 family members of 42 FCHL families, including the 31 FCHL families of the original linkage study⁴. This strategy was not applied for the TXNIP gene the variants of which all had a minor allele frequency<10%. The physical order of the markers and genes was determined using the UCSC Genome Browser. The novel SNPs characterized in this study will be submitted to public databases (NCBI). All SNPs were tested for possible violation of Hardy Weinberg equilibrium (HWE) in three groups (all family members, probands, and spouses) using the HWSNP program developed by Dr. Markus Perola at the National Public Health Institute of Finland. Annotation data of the Alu elements were downloaded from the UCSC Genome Browser, which uses the RepeatMasker to screen DNA sequences for interspersed repeats. The positions of the 60-bp sequence on these Alu elements were identified using the BLAST. Other annotation data were downloaded from the LocusLink.

### Expression Array Analysis of Adipose Tissue

Six affected FCHL family members exhibiting the susceptibility haplotype (see Results) and four affected FCHL family members homozygous for the protective haplotype were selected for assessment of gene expression. All six susceptibility haplotype carriers were from six individual families. The four homozygous protective haplotype carriers were two subpairs from two families. Biopsies were taken from umbilical subcutaneous adipose tissue under local anaesthesia to collect 50-2000 mg of adipose tissue. The RNA was extracted using STAT RNA-60 reagent (Tel-Test, Inc.), according to the manufacturer's instructions, followed by DNAse. I treatment and additional purification with RNeasy Mini Kit columns (Qiagen). The quality of the RNA was assessed using the RNA 6000 Nano assay in the Bioanalyzer (Agilent) monitoring for ribosomal S28/S18 RNA ratio and signs of degradation. The concentration and the A260/A280 ratio of the samples were measured using a spectrophotometer, the acceptable ratio being 1.8-2.2. Then 2 μg of total RNA was reverse transcribed to cDNA using the SuperScript Choice System (Invitrogen) and T7-oligo(dT)₂₄ primer, according to instructions provided by Affymetrix, except using 60 pmols of primer and a reaction volume of 10 μl, after which biotin-labeled cRNA was created using Enzo® BioArray™ HighYield™ RNA Transcript Labeling Kit (Affymetrix). Prior to hybridization the cRNA was fragmented to obtain a transcript size distribution of 50 to 200 bases, after which samples were hybridized to Affymetrix Human Genome U133A arrays and scanned in accordance with the manufacturers' recommendations.

Scanned images were analyzed with Affymetrix Microarray Suite 5 (Affymetrix, Santa Clara, Calif.) software employing the Statistical Expression Algorithm. All analysis parameters were set to the default values recommended by Affymetrix. Global scaling to a target intensity of 100 was applied to all arrays but no further normalizations were performed at this point. Output files of result metrics, including the scaled signal intensity values and the corresponding detection call expressed as absent, marginal or present, were further processed using GeneSpring 5.0 data analysis software (Silicon Genetics, Redwood City, Calif.). For each probe array a per gene normalization was applied so that signal intensities were divided by the median intensity calculated using all 10 probe arrays. Cut-off values to discriminate low quality data were determined separately for each haplotype group by dividing the base value with the proportional value estimated using the Cross Gene Error Model implemented in GeneSpring. To identify differentially expressed genes between the two haplotypes, ratios of averaged normalized intensities were calculated. Differences were considered as significant if the resulting ratio fell at least three standard deviations outside the average ratio calculated from the distribution of the log₁₀ of the ratios. To further increase result stringency only genes scored as present in all 10 samples, or as absent or marginal in all cases and present in all the controls (or vice versa), were included. Annotation information defining the biological processes that each gene could be ascribed to was retrieved from the classifications provided by the gene ontology (GO) consortium⁴¹. Statistical evaluation of enrichment of categories represented in each gene list, compared to the proportion observed in the total population of genes on the probe array, was performed using the Expression Analysis Systematic Explorer (EASE) tool⁴¹, with the threshold value set to 3. The test statistic was calculated using Fisher's exact test. To maximize robustness, an EASE score (p-value) was calculated where the Fisher exact probabilities were adjusted so that categories supported by few genes were strongly penalized, while categories supported by many genes were negligibly penalized. EASE scores (p-values) falling below 0.05 were considered statistically significant.

### Quantitative Real-Time PCR Analysis of USF1

Two affected FCHL family members exhibiting the susceptibility haplotype and two affected FCHL family members without the haplotype were selected for assessment of USF1 expression in adipose tissue utilizing the SYBR-Green assay (Applied Biosystems). Two step RT-PCR was done using TaqMan Gold RT-PCR kit according to manufacturers' recommendations. A total of 1 μg of RNA was converted to cDNA in a 100 μl reaction of which 1 μl was used in the quantitative PCR reaction. The ratio of USF1 to two housekeeping genes GAPDH and HPBGD was used to normalize the data. The specificity of the reaction was evaluated using a dissociation curve in addition to a no-template control. The following PCR primers were used in separate 10 μl SYBR-Green reactions: For USF1; forward: 5′-ATGACGTGCTTCGACAACAG-3′, reverse: 5′-GGGCTATCTGCAGTTCTTGG-3′. For GAPDH; forward: 5′-CGGAGTCAACGGATTTGGTCGTAT3′, reverse: 5′-AGCCTTCTCCATGGTGGTGAAGAC-3′. For HPBGD; forward: 5′-AACCCTCATGATGCTGTTGTC-3′, reverse: 5′-TAGGATGATGGCACTGAACTC3′. The reactions were run in triplicate using the ABI Prism 7900 HT Sequence Detection System in accordance with the manufacturers' recommendations and the data were analyzed using Sequence Detector version 2.0 software.

### Initial Functional Analysis

Initial functional analyses were performed using the SEAP reporter system (Clontech Laboratories, Palo Alto, Calif.) in COS cells. This system utilizes SEAP, a secreted form of human placental alkaline phosphatase, as a reporter molecule to monitor the activity of potential promoter and enhancer sequences. The constructs were cloned into the pSEAP2-Enhancer vector which contains the SV40 enhancer. The correct allele and orientation in each construct was verified by sequencing. Cell culture media between 48 h and 72 h after transfection were taken for the SEAP reporter assay. The monitoring of the SEAP protein was performed using the fluorescent substrate 4-methylumbelliferyl phosphate (MUP) in a fluorescent assay according to the manufacturer's instructions. Data are representative of at least two independent experiments.

### Statistical Analyses

Parametric linkage and nonparametric affected sib-pair (ASP) analyses were carried using the same programs and parameters as in the original linkage study⁴. Two traits were investigated, the FCHL and TG trait. The MLINK program of the LINKAGE package⁴³ version FASTLINK 4.1P⁴⁴⁻⁴⁵ was used as implemented by the ANALYZE package⁴⁶ to perform the parametric two-point and multipoint linkage analyses. The ASP analysis was performed using the SIBPAIR program of the ANALYZE package⁴⁶. For each marker, allele frequencies were estimated from all individuals using the DOWNFREQ program⁴⁷.

The SNPs were tested for association using the HHRR²⁷ and the gamete competition test²⁹. To minimize the number of tests performed, the SNPs residing outside the USF1-JAM1 region were tested for association only using the HHRR²⁷ test when analyzing the TG- and FCHL-affected males. The HHRR analysis, performed by use of the HRRLAMB program⁴⁸, tests the homogeneity of marker allele distributions between transmitted and non-transmitted alleles. The multi-HHRR analysis is testing the same hypothesis using several SNPs. The gamete competition test is a generalization of the TDT and views transmission of marker alleles to affected children as a contest between the alleles, making effective use of full pedigree data. The gamete competition method is not purely a test of association, because the null hypothesis is no association and no linkage, and thus linkage in itself also affects the observed p-value. Furthermore, the gamete competition test readily extends to two linked markers, enabling simultaneous analysis of multiple SNPs in a gene. P-values based on asymptotic approximations can be biased when data used to calculate them are relatively sparse. To confirm that the gamete competition results are indeed significant we also calculated empirical p-values for all analyses involving multiple SNPs (Table 1) using gene dropping. In gene dropping the founder genotypes are assigned using the estimated allele frequencies assuming HWE and linkage equilibrium (LE). The offspring genotypes are assigned assuming Mendelian segregation. Thus gene dropping is performed under the null hypothesis of LE and no linkage. To calculate an empirical p-value, gene dropping is performed multiple times. Here at least 50,000 simulations were performed for each analysis. The likelihood ratio test statistic (LRT) from each gene dropping iteration is compared to the LRT for the observed data. The empirical p-value is the proportion of iterations in which the gene dropping LRT equaled or exceeded the observed LRT. In general, the obtained empirical p-values of gene dropping are more conservative than asymptotic p-values for small sample sizes.

The HBAT program, options optimize offset (-o) and empirical test (-e), were performed to test for association between haplotypes and the trait⁴⁹. The option-o measures not only preferential transmission of the susceptibility haplotype to affected but also less preferential transmissions to unaffecteds. The e option leads to a test of association given linkage and gives thus an empirical estimation of the variance. These haplotype analyses are affected by the fact that four of the 15 SNPs for the JAM1-USF1 region were genotyped in the 60 extended FCHL families and 11 SNPs in 42 nuclear FCHL families. The genotype Pedigree Disequilibrium Test (geno-PDT)⁵⁰, which provides a genotype-based association test for general pedigrees, was also performed for a combination of genotypes from selected USF1 SNPs (Table 3). LD between the marker genotypes for SNPs in the JAM1-USF1 region was tested using the Genepop v3.1b program, option 2, at their web site. In this program, one test of association is performed for genotypic LD, and the null hypothesis is that genotypes, at one locus are independent from the genotypes at the other locus. The program creates contingency tables for all pairs of loci in each population and performs Fisher exact test for each table using a Markov chain.

### URLs

Supplementary Tables 1-4 and further details on microarray data will be available at our web site (www.genetics.ucla.edu/labs/pajukanta/fchl/chr1/). The raw data for the complete set of probe arrays can be accessed through the Gene Expression Omnibus at NCBI (www.ncbi.nlm.nih.gov/geo) using the GEO accession GSE590. The Finnish 90^{th} age-sex specific percentile values for TC and TGs are available at the web site of the National Public Health Institute of Finland (www.ktl.fi.molbio/wwwpub/fchl/genomescan). We used the dbSNP (available at www.ncbi.nim.nih.gov) and CELERA (www.celera.com) for SNP selection; the UCSC Genome Browser (genome.ucsc.edu) for physical order of the genes and for annotation of the Alu element; the BLAST (www.ncbi.nlm.nih.gov/blast/) for blasting sequences against human and mouse databases; the LocusLink (www.ncbi.nim.nih.gov/LocusLink/) to download annotation data; and the Genepop (wbiomed.curtin.edu.au/genepop/index.html) to calculate intermarker LD.

### EXAMPLE 6

### Methods in Examples 7 to 11

### Electrophoretic-Mobility-Shift Assay (EMSA)

DNA probes representing both strands of the regions of interest were ordered from Proligo and 5′-end-labeled with [γ-32P]ATP using T4 polynucleotide kinase. Excess unincorporated label was removed using the QIAquick kit (Qiagen) according to manufacturer's instructions. Nuclear extracts were incubated for 30 minutes at room temperature in binding buffer (50 mM Tris-HCl (pH 7.5), 5 mM MgCl₂, 2.5 mM EDTA, 2.5 mM DTT, 2.5 mM NaCl, 0.25 μg/μl poly(dl-dC).poly(dl-dC), 20% glycerol) and then electrophoresed on a 6% polyacrylamide gel containing 0.5 M TBE buffer. Gels were autoradiographed at −70° C. In order to test for specificity of binding, the extracts were run with an increasing concentration of unlabeled “cold” ds-probe as well as non-specific probe representing the sequence around the 3′-UTR SNP usf1 μl that did not produce a gel shift.

### Expression Array Analysis

We selected 19 individuals for fat biopsy from our FCHL (ref. 6A) and low-HDL-C families^{33A} based on their USF1 haplotype. They included 12 carriers of the risk-allele of the critical SNP usf1s2 and 7 individuals homozygous for the non-risk allele. Nine of these had been included in our original report^{6A}. The average age in both groups was 49 years and the gender distribution was close to even (7 females and 5 males in the risk group versus 4 females and 3 males in the non-risk group). Fat biopsies were collected, RNA extracted and quantified as described previously^{6A}. RNA labeling, array processing and scanning was done according to the standard protocol by Affymetrix with minor modifications, as described previously^{6A}.

Scanned images were analyzed with Affymetrix Microarray Suite 5 (Affymetrix, Santa Clara, Calif.) software employing the Statistical Expression Algorithm. Global scaling to a target intensity of 100 was applied to all arrays, after which further data processing was carried out using GeneSpring 6.1 data analysis software (Silicon Genetics, Redwood City, Calif.). For each probe array, we applied a per gene normalization so that signal intensities were divided by the median intensity calculated using all 19 probe arrays, effectively centering the data around unity.

To identify differentially expressed genes between the two haplotypes, we adopted a strategy consisting of two filtering steps, in combination with a statistical analysis. First, we removed unreliable or inconsistent data using the Affymetrix detection calls, requiring genes to be scored as present in more than 50% of the samples in each haplotype group. In order to avoid losing potentially interesting data pertaining to genes whose expression was “turned off” in one group but “turned-on” in the other, we also included genes scoring absent calls in 100% of samples in one group and at least 50% present calls in the other. Normalized values were then averaged over samples in each haplotype group and ratios of these were calculated. The distribution of the ratios was evaluated and a cut-off limit of 1.5 fold was selected to focus attention on the most prominent and reliable expression changes. We determined significant changes by applying a two-sample t-test, allowing for unequal variances across groups, where a two-sided. P-value of 0.05 or lower was considered statistically significant. For the genes represented by more than one probe set on the array the measurements associated with the more conservative P-value were used.

### Statistical Analyses

We evaluated the effect of haplotype on gene expression for selected genes using a two-sample t-test, with no assumption of equal variances. Two-sided significance values were calculated and a type I error probability of 5% or lower was used to determine statistical significance. To control for possible confounding contribution from clinically relevant parameters on the observed differences between haplotype groups, we performed analyses of co-variance (ANCOVA). BMI, levels of insulin and triglycerides and HOMA index were included as co-variates to the factor determined by haplotype group and separate models for each co-variate were evaluated for main and interaction effects. Again, we considered type I errors at a probability of 5% or lower statistically significant. Closer scrutiny of haplotype effects on the relationship between gene expression and co-variates was done by linear regression analysis. The linear models were evaluated studying R, R² and the F statistic.

Unsupervised hierarchical clustering of samples with respect to patterns of gene expression for selected genes was performed employing an agglomerative algorithm using unweighted pair-group average linkage, UPGA, amalgamation rules. Cluster similarity was determined with Pearsons' correlation. We analyzed possible associations between branching pattern and gender, affection status (FCHL or low-HDL) and familial relationships by overlaying status information on the dendrogram and visually assessing potential clusters.

### EXAMPLE 7

### Critical Intronic Sequence Binds Nuclear Protein

Among the nine identified intragenic USF1 SNPs, two represent synonymous variants in the coding region, while seven were located in introns (FIG. 4a). The strongest evidence for association in FCHL families was initially observed with two SNPs: usf1s1 in the 3′-UTR, and usf1s2 in intron 7, located 1.24 kb apart and essentially in complete LD (D′=0.98). We analyzed the sequence environment of all 7 intronic SNPs across species to monitor for phylogenetic conservation that would provide clues of their functional importance. The strongest associating SNP usf1s2 in intron 7 was located in a DNA stretch fully conserved from human through chimp, dog mouse and rat, within a genomic region otherwise rich in non-conserved nucleotides (FIG. 4b). The only other SNP to be located in such a conserved sequence stretch was usf1 s9 in intron 1, but since it revealed no association with FCHL or it's component traits, we did not pursue it further. The regional conservation of this sequence containing usf1s2 encouraged us to study whether it harbored some elements functionally important to the dynamics of USF1 transcription.

We first determined whether the region of usf1s2 represents a binding site for DNA binding proteins. We constructed two 34-mer probes (FIG. 4b) containing SNPs usf1s2-4 and allowed them to vary for the two alleles of usf1s2. After incubation with nuclear extract proteins of HeLa cells, both critical sequence variants produced an electrophoretic mobility shift (EMS) on a polyacrylamide gel. To further restrict the potentially functional sequence motif, we performed the EMS analyses using a shorter, 20-mer probe pair that shared with the 34-mer probe the critical most conserved nucleotide sequence. This probe produced a mobility shift, comparable to the 34 bp shift, whereas a similar 20 bp probe representing the sequence containing the other strongly associated SNP usf1s1, located in the 3′UTR of USF1 did not produce a shift (FIG. 5a). The binding of the probes to nuclear proteins could be competed using unlabeled specific probe, but not with a non-specific probe (FIG. 5b).

### EXAMPLE 8

### Carriers of USF1 Risk Allele Show Differential Expression of Downstream Genes in Fat

A qualitative or quantitative functional change of a transcription factor such as USF1 would be expected to be reflected in the expression efficiency or pattern of the genes under its control. We hypothesized that if the usf1s2 polymorphism either itself was functional or served as a marker for an unknown functional element in the vicinity, we should be able to see a difference in the transcriptional profile of USF1 regulated genes in fat biopsies of individuals carrying either the “risk” or “non-risk” allele. This would represent an eloquent in vivo approach to address the function of the potential susceptibility polymorphism. We made a query of a transcription factor database (Transfac) and published literature and identified a total of 40 USF1-controlled genes and selected them for further analysis regardless of knowledge over biological pathway or tissue specificity (Table 4).

TABLE 4
GENES WITH REPORTED INVOLVEMENT OF USF1 IN THEIR REGULATION

| Gene | | On the | Expressed in |
|---|---|---|---|
| Symbol | Full Name | U133A chip | fat biopsies |
| APOC3 | Apolipoprotein C-III | X | |
| APOA2 | Apolipoprotein A2 | X | |
| APOA5 | Apolipoprotein A5 | | |
| APOE | Apolipoprotein E | X | X |
| LIPE | Hormone sensitive lipase | X | X |
| Spot-14 | Spot 14 protein | | |
| FAS | Fatty acid synthase | X | |
| ABCA1 | ATP-binding cassette, subfamily A | X | X |
| ACACA | Acetyl-CoA carboxylase alpha | X | X |
| GHRL | Ghrelin | | |
| GCK | Glucokinase | X | |
| GCGR | Glucagon receptor | X | |
| REN | Renin | X | |
| AGT | Angiotensinogen | X | X |
| FSHR | Follicle stimulating hormone receptor | X | |
| HOXB4 | Homeobox B4 | | |
| MHC I | Major Histocompatibility Complex I | | |
| HOXB7 | Homeobox B7 | X | X |
| HBB | Human beta-globin | X | X |
| MAP2K1 | Mitogen-activated protein kinase phosphatase 1 | X | X |
| CCNB1 | Cyclin B1 | X | X |
| L-PK | L-type pyruvate kinase | X | |
| NCA | Non-specific cross reacting antigen | X | |
| EFP | Estrogen responsive finger protein | | |
| OPN | Osteopontin | X | X |
| TRAP | Tartrate resistant acid phosphatase | | |
| BDNF | Brain Derived Neurotrophic Factor | | |
| PAI-1 | Plasminogen activator inhibitor type 1 | X | |
| FceRI | High-affinity IgE receptor | | |
| BRCA2 | Hereditary breast cancer susceptibility gene 2 | X | |
| dCK | Deoxycytidine kinase | X | |
| PIGR | Polymeric immunoglobulin receptor | X | |
| CYP19 | Cytochrome P450, Family 19 | X | |
| hTERT | Human telomerase reverse transcriptase | | |
| PF4 | Platelet factor 4 | X | |
| CDK4 | Cyclin-dependent kinase 4 | X | X |
| CYP3A4 | Cytochrome P450, family 3A, polypeptide 4 | X | X |
| SHP-1 | Protein-tyrosine phosphatase with two src-homology 2 | | |
| | domains | | |
| FMR-1 | Fragile X Mental Retardation | X | X |
| CYP1A1 | Cytochrome P450, family 1, subfamily A, polypeptide 1 | X | |
| | 40 | 29 | 13 |

USFs have been reported to bind promoters of these genes either in vitro or in vivo and for several there is functional evidence. A complete list of references is available upon request. Of these genes, 29 were represented on the Affymetrix U133A chip used in this study. 13 were expressed in the fat biopsies at a level that produced reliable signal. The genes in bold were statistically significantly differentially expressed between individuals carrying different alleles of usf1s2.

To study the possible effects of allelic variants of USF1 on the transcriptional profiles, we obtained fat biopsies from 19 individuals from our cohort of dyslipidemic families (FCHL and low-HDL-C). They included 7 individuals homozygous for the rare 2-2 genotype of usf1s2 (marking the “non-risk” haplotype) and 12 individuals carrying the common 1 allele (marking the “risk” haplotype) in either heterozygous (8) or homozygous (4) form. Out of 40 listed USF1-controlled genes, 29 were represented on the Affymetrix U133A chips used in this study, some genes by multiple probe sets. We found that 13 genes, represented by a total of 19 probe sets, were expressed in the adipose tissue at a sufficiently high level as to produce reliable signals and were included in the study (Table 4). Several highly, relevant genes of lipid and glucose metabolism were on this list as well as a few genes whose relevancy isn't immediately obvious. After normalization, three genes (represented by a total of 6 probe sets all in agreement) differed significantly (P≦0.05) in their expression between the two haplotype groups of USF1, as evaluated using a two-sample t-test with no assumption of equal variance. All three genes, differentially expressed between individuals carrying either the “risk” or “non-risk” haplotype of USF1, were highly relevant to the phenotype: the ATP-binding cassette subfamily A (ABCA1) (ref. 13A), angiotensinogen (AGT) (ref. 14A) and apolipoprotein E (APOE) (ref. 15A) (FIG. 7).

### EXAMPLE 9

### Differential Response of ACACA to Insulin

Signals such as serum insulin and glucose are critical in the regulation of various metabolic genes. Insulin is known to influence the ability of USF1 to bind the E-box sequence and thus participate in the regulation of gene expression in response to metabolic changes^{16A}. To evaluate the possible contribution of these factors on the expression of the USF1-controlled genes, we fitted ANCOVA models to the data. We further extended the models to also test for possible effects of body mass index (BMI), triglycerides and HOMA (homeostatic model assessment), a measure of insulin resistance based on values for fasting serum insulin and glucose^{17A}. For all but one of the genes tested, we observed no significant contribution from the various covariates, hence resulting in test statistics essentially the same as those of the simple, two-sample t-test. However, in agreement with earlier findings^{18A} we observed a detectable effect of the insulin level on the expression of acetyl-CoA carboxylase alpha (ACACA) (P=0.05). This relationship, was closer scrutinized using linear regression, which demonstrated a moderately strong negative correlation (R²=0.453) between the steady state transcript level of ACACA and fasting levels of insulin. Partial regression for the haplotype groups additionally demonstrated that this correlation was in essence much stronger in the individuals with the 2-2. “non-risk” haplotype (R²=0.956) than in individuals carrying the “risk” haplotype (R²=0.093) of USF1.

We also tested whether any effect of parameters like sex or study cohort (FCHL or low-HDL) should be taken into account in our analyses by performing an unsupervised clustering of individual expression levels. We detected no effect for any measures looked at, as evidenced by the random clustering of individuals with respect to these variables (data not shown).

### EXAMPLE 10

### Changes in APOE Stand Out in Whole Genome Transcript Profile

In addition to the analyses of known USF1-regulated genes, we tested the whole micro-array data for altered transcript levels of genes between carriers of the different USF1 haplotypes. Approaches of this kind have been successfully used to identify pathways and collections of co-regulated genes in different sets^{19A}. This has most often been done when comparing groups with a clear phenotypic difference such as diabetic vs. non-diabetic^{19A}, or cancer tissue vs. non-cancerous tissue.^{20A} In our study, changes in which the expression differences were ≧1.5 fold, and that reached our limit of statistical significance (P≦0.05) in the two-sample t-test were defined as significant. This approach identified fifteen genes, among which 10 were upregulated and 5 downregulated in individuals with the non-risk haplotype (Table 5).

TABLE 5
MOST DIFFERENTIALLY EXPRESSED GENES ACROSS
ENTIRE ARRAY

| | | | |
|---|---|---|---|
| Common | Genbank ID | Fold change | P-value |

Up regulated in non-risk individuals

| | | | |
|---|---|---|---|
| APOE | N33009 | 2.0 | 0.0163 |
| MBD4 | AI913365 | 1.9 | 0.0293 |
| GLUL | NM_002065 | 1.8 | 0.0473 |
| ESTs | AA721025 | 1.7 | 0.0471 |
| CYP4B1 | J02871 | 1.6 | 0.0200 |
| VEGF | AF022375 | 1.6 | 0.0174 |
| SLC6A8 | U17986 | 1.6 | 0.0121 |
| CIDEA | NM_001279 | 1.6 | 0.0229 |
| LY75 | NM_002349 | 1.5 | 0.0298 |
| FLJ20859 | NM_022734 | 1.5 | 0.0001 |

Down regulated in non-risk individuals

| | | | |
|---|---|---|---|
| TNMD | NM_022144 | −2.2 | 0.0083 |
| DKFZP761N09121 | BF435376 | −1.7 | 0.0029 |
| IL6 | NM_000600 | −1.6 | 0.0024 |
| AGTRL1 | X89271 | −1.6 | 0.0186 |
| TYRP1 | NM_000550 | −1.5 | 0.0240 |

Comparing the normalized gene expression across the entire array between the two haplotype groups (as defined by the allele at usf1s2) was used to generate a list of the most differentially regulated genes. A significant change was defined as one in which the expression differences were at least 1.5 fold, and that reached our limit of statistical significance (P ≦ 0.05) in the two-sample t-test. Notably the most up regulated gene in non-risk individuals was the USF1-regulatedgene apolipoprotein E.

Again, the top gene on the list of downregulated genes in the risk individuals was APOE. The expression of APOE in the adipose tissue of individuals with the risk haplotype of USF1 was twice as low as expression in those carrying the non-risk haplotype. Other potentially interesting genes on the list included CYP4B1, involved in fatty acid metabolism, and VEGF, involved in angiogenesis, hypertension and it is an essential mediator in angiotensin II induced vascular inflammation^{21A}. Experimental data is needed to verify whether USF1 plays a role in the regulation of these genes as well.

### EXAMPLE 11

### No Strong Effect of Critical SNP on Regional Genes

Finally, to investigate whether the putative regulatory element in intron 7 could represent a strong cis-regulatory element and exert its control on the expression of other genes in the vicinity of USF1, we studied the expression levels of 10 flanking genes from the 5′ CD244 gene all the way to APOA2, a stretch of 392 kb. Of these 10 genes, 6 are transcribed from the same DNA strand as USF1 and 4 from the opposite strand. The only probe set whose expression level differed significantly depending on an individual's allele at usf1s2 was one for the adjacent platelet F11 receptor (F11R) gene (P=0.013). This was interesting since the critical chromosomal interval showing an association in FCHL families reached into the F11R gene in alleles of high-triglyceride men^{6A}. On the U133A array two probe sets represent F11R, however only one showed significant difference between the two USF1 haplotype groups. Upon closer examination of the representative sequence in the genome, we noted that the probe set which showed differential expression did not actually represent the F11R gene, but rather a short expressed sequence tag (EST) (AAW995043) immediately adjacent to it, 43.5 kb 3′ from the USF1 gene.

1. A nucleic acid molecule comprising a chromosomal region contributing to or indicative of hyperlipidemias and/or dyslipidemias and/or defective carbohydrate metabolism, wherein said nucleic acid molecule is selected from the group consisting of:
(a) a nucleic acid molecule having or comprising the nucleic acid sequence of SEQ ID NO: 1, wherein said nucleic acid sequence has one or more mutations having an effect on USF1 function;
(b) a nucleic acid molecule having or comprising the nucleic acid sequence of SEQ ID NO: 1, wherein said nucleic acid sequence is characterized by comprising a guanine or an adenine residue in position 3966 in intron 7 of the USF1 sequence; and/or
(c) a nucleic acid molecule having or comprising the nucleic acid sequence of SEQ ID NO: 1, wherein said nucleic acid-sequence is characterized by comprising a cytosine or a thymine residue in position 5205 in exon 11 of the USF1 sequence;
wherein said nucleic molecule extends, at a maximum, 50000 nucleotides over the 5′ and/or 3′ end of the nucleic acid molecule of SEQ ID NO: 1.

2. The nucleic acid molecule of claim 1 which is genomic DNA.

3. A fragment of the nucleic acid molecule of claim 1 or 2 having at least 20 nucleotides wherein said fragment comprises nucleotide position 3966 and/or position 5205 of SEQ ID NO:1.

4. A nucleic acid molecule which is complementary to the nucleic acid molecule of any one of claims 1 to 3 and which has a length of at least 20 nucleotides.

5. A vector comprising the nucleic acid molecule of any one of claim 1 to 4.

6. A primer or primer pair, wherein the primer or primer pair hybridizes under stringent conditions to the nucleic acid molecule of any one of claims 1 to 4 comprising nucleotide positions 3966 and 5205 SEQ ID NO:1 or to the complementary strand thereof.

7. A non-human host transformed with the vector of claim 5.

8. The non-human host of claim 7 which is a bacterium, a yeast cell, an insect cell, a fungal cell, a mammalian cell, a plant cell, a transgenic animal or a transgenic plant.

9. A pharmaceutical composition comprising USF1 or a fragment thereof, a nucleic acid molecule encoding USF1 or a fragment thereof, or an antibody specific for USF1.

10. A diagnostic composition comprising a nucleic acid molecule encoding USF1 or a fragment thereof, the nucleic acid molecule of any one of claims 1 to 4, the vector of claim 5, the primer or primer pair of claim 6 or an antibody specific for USF1.

11. A method for testing for the presence or predisposition of hyperlipidemia and/or dyslipidemia and/or defective carbohydrate metabolism, comprising analyzing a sample obtained from a prospective patient or from a person suspected of carrying such a predisposition for the presence of a wild-type or variant allele of the USF1 gene.

12. The method of claim 11, wherein said variant comprises an SNP at position 3966 and/or at position 5205 of the USF1 gene in a homozygous or heterozygous state.

13. The method of claim 11 or 12, wherein said testing comprises hybridizing the complementary nucleic acid molecule of claim 4 under stringent conditions to nucleic acid molecules comprised in a sample and detecting said hybridization, wherein said complementary nucleic acid molecule comprises the sequence position containing the SNP.

14. The method of any one of claim 11 to 13 further comprising digesting the product of said hybridization with a restriction endonuclease or subjecting the product of said hybridization to digestion with a restriction endonuclease and analyzing the product of said digestion.

15. The method of claim 14, wherein said probe is detectably labeled.

16. The method of any one of claims 11 to 15, wherein said testing comprises determining the nucleic acid sequence of at least a portion of the nucleic acid molecule of any one of claims 1 to 4, wherein said portion comprises the position of the SNP.

17. The method of claim 16, wherein the determination of the nucleic acid sequence is effected by solid-phase minisequencing.

18. The method of claim 17 further comprising, prior to determining said nucleic acid sequence, amplification of at least said portion of said nucleic acid molecule.

19. The method of claim 11 to 15, wherein said testing comprises carrying out an amplification reaction wherein at least one of the primers employed in said amplification reaction is the primer of claim 6 or belongs to the primer pair of claim 6, comprising assaying for an amplification product.

20. The method of claim 19 wherein said amplification is effected by or said amplification is the polymerase chain reaction (PCR).

21. A method for testing for the presence or predisposition of hyperlipidemia and/or dyslipidemia and/or defective carbohydrate metabolism comprising assaying a sample obtained from a human for the amount of (a) USF1, (b) ABCA1, (c) angiotensinogen or (d) apolipoprotein E contained in said sample.

22. The method of claim 21, wherein said testing is effected by using an antibody or aptamer specific for (a) USF1 (b) ABCA1, (c) angiotensinogen or (d) apolipoprotein E.

23. The method of claim 22, wherein said antibody or aptamer is detectably labeled.

24. The method of any one of claims 21 to 23, wherein the test is an immunoassay.

25. A method for testing for the presence or predisposition of hyperlipidemia and/or dyslipidemia and/or defective carbohydrate metabolism comprising assaying a sample obtained from a human for the amount of RNA encoding (a) ABCA1, (b) angiotensinogen or (c) apolipoprotein E contained in said sample.

26. The method of any one of claims 11 to 25, wherein said sample is blood, serum, plasma, fetal tissue, saliva, urine, mucosal tissue, mucus, vaginal tissue, fetal tissue obtained from the vagina, skin, hair, hair follicle or another human tissue.

27. The method of any one of claims 11 to 26, wherein the nucleic acid molecule or protein from said sample is fixed to a solid support.

28. The method of claim 27, wherein said solid support is a chip, a silica wafer, a bead or a microtiter plate.

29. Use of the nucleic acid molecule of any one of claims 1 to 5 for the analysis of the presence or predisposition of hyperlipidemia and/or dyslipidemia and/or defective carbohydrate metabolism.

30. Use of USF1 or a fragment thereof or of a nucleic acid molecule encoding USF1 and/or comprising at least the wild-type sequence of intron 7 and/or exon 11 of USF1, for the preparation of a pharmaceutical composition for the treatment of hyperlipidemias and/or dyslipidemias including familial combined hyperlipidemia (FCHL), hypercholesterolemia, hypertriglyceridemia, hypoalphalipoproteinemia, hyperapobetalipoproteinemia (hyperapoB), familial dyslipidemic hypertension (FDH), metabolic syndrome, type 2 diabetes mellitus, coronary heart disease, atherosclerosis or hypertension.

31. Kit comprising the nucleic acid molecule of any one of claims 1 to 5, the primer or primer pair of claim 6 and/or the vector of claim 7 in one or more containers.

32. Use of an inhibitor of expression of USF1, wherein said inhibitor is (a) an siRNA or antisense RNA molecule comprising a nucleotide sequence complementary to the transcribed region of the USF1 gene or (b) of an antibody, aptamer or small inhibitory molecule specific for USF1, for the preparation of a pharmaceutical composition for the treatment of hyperlipidemias and/or dyslipidemias including familial combined hyperlipidemia (FCHL), hypercholesterolemia, hypertriglyceridemia, hypoalphalipoproteinemia, hyperapobetalipoproteinemia (hyperapoB), familial dyslipidemic hypertension (FDH), metabolic syndrome, type 2 diabetes mellitus, coronary heart disease, atherosclerosis or hypertension.

33. Use of an activator of expression of USF1 for the preparation of a pharmaceutical composition for the treatment of hyperlipidemias and/or dyslipidemias including familial combined hyperlipidemia (FCHL), hypercholesterolemia, hypertriglyceridemia, hypoalphalipoproteinemia, hyperapobetalipoproteinemia (hyperapoB), familial dyslipidemic hypertension (FDH), metabolic syndrome, type 2 diabetes mellitus, coronary heart disease, atherosclerosis or hypertension, wherein said activator is a small molecule.


SEQUENCE LISTING (15 sequences)

SEQ ID NO: 1 - DNA, 5687 bp, Human
  variation (3966)..(3966): r = adenine (a) or guanine (g) adenine is wild-type associated; guanine is disease-associated
ttgaaaattttccttggataggaaaggtttggaggaccttatgggtagagaatttccaaaaatcttgccccttttgtgttgggattatcttattgctttgtactgtgtagctgtttctttctggaggcatgtctgcccagctctttgtttttcctgccctctggctgggtgtcagggtcctaaggcagagcttgtaggtggattcttccccctttgtctcttcttcagaaccctgttttttttttttttaccccttcttgctcaggcttagttgatttggagttgtcatagcaacattttagcaacagtgttgttctgcaggaaggcttgatgaataaaatagagaatgcttgaagaggatccacttgggctttagggtttctaacagattatataaatctggataccccaaaacaagagtcctgtcagtagaatggggcccaaatgccaagtctagtctttgtggtcagggatattcttccagtggtagtgggcttcagatttcctcttcctaggtttgaaaacagaaatgtcttgatggacaacatgtggctgagaaactggaagaagcatcagtgtccatgacactgtattttttgatggtggggccaatacatggcccttcctgattcccatgaagctgccatcatggcaggtcataatagctttaatgatccatttagagatgtgttgttggctgggtgcggtggctcatgcctgtaatccaagcactttgggaggccgaggcaggcggatcacctgaggtcaggagttccagaccagcctggccaatatggtaaaaccccatctctactgaaaatacaaaaattagctgggcgtggtggtgggcacctataatcccagctattcaggaggctgaggcaggagaatcacttgaacccaggagatggaggttgtaagccgagattgtgccactgcactccagcctgggtgacagagcaagattctgtctcagaaaaaaaaaaaaaaaaaagaaagaaatgtgttgtttcggccaggtgcagtggctcacacctgtaatcccagcactttgggaggctgccgaggtggacagatcatgctctcaggagttcgagaccagccgggccaacatggtgaaaccccgtctctactaaaaatacaaaaattagccaggcgtggtggtgtgcacctgtaatcccagctactccggaggctgaggcaggagaatcacttgaacctgggaggcagaggttgcagtgagctgagatcgcgccactgcactccagcctgggtgacagagagagactctgtctcaaaaaaaaaaaaaaaaaaaaagtgttgtttctgtcttccagtataattatccactctccaccaggagttggagtgataatggagggatggggaacactatttgtagccttgctttttcaatcactgtaggccagtcctcaacatcagtatggtggaggctgattgtcccctgcagatgactgggttattttcctggctatgtgttcatggaacctaagttctagaaccagagatactgttctgtttcctaaactcattgcaaacttcatgatttctaccaggacttagcactcaggcctgtgaatcaggagatacaaagacctccaaaaaaggaccagttcctcggatgtgccccctcacagagagatgaaggggtgagtgaagaagaggtagggtctgggatgaaagatgggtggcctggaagaatgcaaaatgaccaagagcactgcctctggagtcaggcagacctggattcaggttctactctatcacttactgtgtgatttggtttctctatctataaaatggaagtagtgctatctatctcgtggtgctgtttttagtactaaataagattacatgtaatgtacttagcttagtgcttatgtacatagtaaacagtaaacactagttgttattctaacctaacccagcttctgttgggaatgccaatgagtttgcagccatatgttactgggccagtgagcttctcattgacttcttctcatactcttccttttgtcctttcaccacaaacaggcagcagaaaacagctgaaacggaagaggggacagtgcagattcaggaaggtgagtgctagaaacagaaccaagactaagaacccatcatggcctcccttccttccccaccagaccatctcctgtgcatcctcctccttccgtgacatgcaaatggaacgggggtagaaaggcagttaactcacagacttttcctttgttcttttaattcaggtgcagtggctactggggaagacccaaccagtgtggctattgccagcatccagtcagctgccaccttccctgaccccaacgtcaagtacgtcttccgaactgagaatgggggccaggtaagggagggggccaggtggctgcaggtgttatctggggttgggattgagggaggtaattgaacatgtcttggggagacctggcttggaggatgagttgaaagagtggactgttgcaggggagggaggtgctaatactggagtagagactggtgtgaggttagatgtatgctgaaacctctgtgtggggaaagaagggagaatggctgaatccatgtctctgaaggactttgttttggggccctatccaagggaagctttatgaggggccctaggattcccaacacttaatcttttcttctctcttcactccctctgccttcctctacacttctaggtgatgtacagggtgatccaggtgtctgaggggcagctggatggccaaactgagggaactggcgccatcagtggctaccctgccactcaatccatgacccaggtacagggtatgggctggggaggtcactagagttctgagaagtaagatgaagaagggaatcagtaggatgggggtgaagctaggaacagtgaggcatctaaggctgccttgtcccaaagcactaggctctccttttctggatgtttctctctctctctctctctctctccaccctacctaccaccccaacggatagaagctgcagagtggtgtagtgggaagaagtttttgactgttaccagaatcagttttcttgctccccttcccaggcggtgatccagggtgctttcaccagtgatgatgcagttgacacggaggggacagctgctgagacgcactatacttacttccccagcacggcagtgggagatggggcagggggtaccacatcggggagtacagctgctgttgttactacccagggctcagaggcactgctggggcaggcgacccctcctggcactggtgagatattgcatgaggatgctggctgaaagggctagaataggctgtgggacatgactggtaggcagtgagccttcactcatgactcttagtgatcattaagacctggacaggcagtgagtctggggctgctcttctattagcatgttctttttagaggaggggaccagggtcttcacctcagggcttggtgaggttcctacccatgtcctgacagaacctaccctgcatcttcacaggtcaattctttgtgatgatgtcaccacaagaagtactgcagggaggaagccagcgctcaattgcccctaggactcacccttattccccgtgagtgacccttgtttcttctcagattccgtaagtggttttttttttttttttttttttttgagacagagtcttgctctgtcacccaggctggagtgcagtggcatgatctcagctcactgcaacctctgcttccagggttcaagcgtttctcatgcctcagcctcctgagtagctggaactacagacatgtaccaccacccctggctaatttttgtatctttagtagagacagggtttcaccatgttggccaggctggtctcgaactcctgacctcaagtgatccgcctgcctcggcctcccaaagtgctgggattacaggtgtgagacaccacacctagctaccataartggtcctaatacctgctaaatcttgtataattccttaaccccaaacttcaatcatgtattttgtcttcttactctggccaccctgggctctgttgtcaggaagtcagaagctccccggacgactcgggatgagaaacgcagggctcagcataatgaaggtaggtatgatctgggtggagctagaagctgtctggtgtgatctcagcagtgatgtctgaggggaggagggattaggtaattttaccctgggacttgtggcgagttttcactgagtcaccttgtcctccactttgccccacagtggagcgtcgccgccgagacaagatcaacaactggatcgtgcagctctccaagataatcccagactgctctatggagagcaccaagtctggccaggtcatggaaagaccctggtagtgggcaggatgcctgaattctgcctcctggtattgtttccagaaatggtagagagaggggcacacatgacagtagtcttatctctccctgaggttcctgtatccctgggagatattataccaccttccttagatgaaaatgaggtccaaagtgtgaacctacttttggaaagcaagctgggtatctgaaatcctagttctcattttgttgaccttatcttgcagagtaaaggtgggattctatccaaagcttgtgattatatccaggagcttcggcagagtaaccaccgcttgtctgaagaactgcagggacttgaccaactgcagctggacaatgacgtgcttcgacaacaggtcagactcctacccccagtgcagcccttctcagttctgctagccactgacccagtttgacaccctctactttgttctccatggagaaggcttcatcttttccccctcaccagtggatgtctgaatacattcaggggcttggaagtgccagctttactacccattccctttactgcctccttcccatgtcaggtggaagatcttaaaaacaagaatctgctgcttcgagctcagttgcggcaccacggattagaggtcgtcatcaagaatgacagcaactaactatggggattcaggggctttgggcccaagaactgcagatagcccaggagcaacagcctaatcccgtgcccctttccttcactgccccacttctggcatgggacagggggaagttcagaaggtgtgtccttgaactgaggccctgtgatatggcggcctgcagtggtgtgaaacacacaatgtggaygtgcactgacagccttgcccacccccaccatgcagcccctgggcccttgtgctcctctcgcacaatgcatgtgctgtctccatgctggatactggacacactaaactctggggcttgtcctgtgcttgcttagagtgcccagcagaggtttgctgacaggtgatgctctggcttgccccaggactctggcacttccattggttcttcctttccctggagctgaggtttagatgtgcaacctgtggctcaggggagcaagcttacacaagaagtgagggaaggatgtttagcagtggctggtgcccatgaagaggagattggccagtgagaagctgaggcctatgcagacatctctggagccagagagaacaacaggcaggggcccacttggggccttcccccttgtgggggtcgttttttttttttcttttcttttttttttttttttttttttttttttaagataaaattgttcaaagcca

SEQ ID NO: 2 - DNA, 56 bp, Homo sapiens
tttagcaggtattaggaccacttatggtagctaggtgtggtgtctcacacctgtaa

SEQ ID NO: 3 - DNA, 53 bp, Artificial Sequence
  Description of Artificial Sequence  Synthetic oligonucleotide probe
tttagcaggtattaggaccattatggtagctggtgtggtgtctcacactgtaa

SEQ ID NO: 4 - DNA, 23 bp, Homo sapiens
ggtattaggaccattatggtagc

SEQ ID NO: 5 - DNA, 23 bp, Chimp
ggtattaggaccattatggtagc

SEQ ID NO: 6 - DNA, 10 bp, Dog
ggatagacca

SEQ ID NO: 7 - DNA, 11 bp, Mouse
ggaaccattac

SEQ ID NO: 8 - DNA, 14 bp, Rat
ggataccatcatac

SEQ ID NO: 9 - DNA, 16 bp, Artificial Sequence
  Description of Artificial Sequence  Consensus sequence from multiple organisms
ggyayyrrraccacyy

SEQ ID NO: 10 - DNA, 20 bp, Artificial Sequence
  Description of Artificial Sequence  Synthetic PCR primer
atgacgtgcttcgacaacag

SEQ ID NO: 11 - DNA, 20 bp, Artificial Sequence
  Description of Artificial Sequence  Synthetic PCR primer
gggctatctgcagttcttgg

SEQ ID NO: 12 - DNA, 24 bp, Artificial Sequence
  Description of Artificial Sequence  Synthetic PCR primer
cggagtcaacggatttggtcgtat

SEQ ID NO: 13 - DNA, 24 bp, Artificial Sequence
  Description of Artificial Sequence  Synthetic PCR primer
agccttctccatggtggtgaagac

SEQ ID NO: 14 - DNA, 21 bp, Artificial Sequence
  Description of Artificial Sequence  Synthetic PCR primer
aaccctcatgatgctgttgtc

SEQ ID NO: 15 - DNA, 21 bp, Artificial Sequence
  Description of Artificial Sequence  Synthetic PCR primer
taggatgatggcactgaactc